(19) United States
(12) Patent Application Publication
(10) Pub. No.: US 2009/0053167 A1
(43) Pub. Date: Feb. 26, 2009

(54) C-, S- and N-glycosylation of peptides

(75) Inventor: Shawn DeFrees, North wales, PA (US)

Correspondence Address: MORGAN, LEWIS & BOCKIUS LLP (SF), One Market, Spear Street Tower, Suite 2800, San Francisco, CA, 94105, US

(73) Assignee: Neose Technologies, Inc., Horsham, PA (US)

(21) Appl. No.: 12/152,587

(22) Filed: May 14, 2008

Related U.S. Application Data
(60) Provisional application No. 60/917,857, filed on May 14, 2007.

(51) Int. Cl. A61K 38/20; C12N 9/12; A61K 38/45; A61K 39/395; C12P 21/02; C07K 16/22; C07K 14/55

(52) U.S. Cl. 424/85.2; 435/194; 424/94.5; 424/145.1; 530/388.24; 530/351; 435/68.1

(57) ABSTRACT

The present invention provides polypeptide conjugates wherein a modifying group such as a water-soluble polymer, a therapeutic agent or a biomolecule is covalently linked to the polypeptide through a glycosyl linking group. In one embodiment, the polypeptide includes a glycosylation consensus sequence, wherein glycosylation occurs at an aromatic amino acid residue, such as the C-2 or the N-1 position of a tryptophan side chain. Exemplary polypeptides of the invention are those in which the glycosylation consensus sequence has been introduced into the amino acid sequence of the polypeptide by mutation. In another aspect the invention provides polypeptide conjugates wherein the modifying group is covalently linked to the polypeptide via a glycosyl mimetic linking group. Also provided are methods of making and using as well as pharmaceutical compositions containing the polypeptide conjugates of the invention. Further provided are methods of treating, ameliorating or preventing diseases in mammals by administering an amount of a polypeptide conjugate of the invention sufficient to achieve the desired response.

### CROSS-REFERENCE TO RELATED APPLICATIONS

The present application claims priority under 35 U.S.C. §119(e) to U.S. Provisional Patent Application No. 60/917,857 filed May 14, 2007, the disclosure of which is incorporated herein by reference for all purposes.

### FIELD OF THE INVENTION

The invention pertains to the field of peptide modification by glycosylation. In particular, the invention pertains to a method of preparing glycosylated peptides using short enzyme-recognition sequences.

### BACKGROUND OF THE INVENTION

The present invention relates to glycosylation and modification of peptides, preferably mutant peptides of therapeutic value that include one or more glycosylation consensus sequence, wherein the consensus sequence includes an aromatic amino acid, which is the site of glycosylation. The consensus sequence is recognized by an enzyme and is typically not present in a corresponding parent or wild-type peptides.

The administration of glycosylated and non-glycosylated peptides for engendering a particular physiological response is well known in the medicinal arts. For example, both purified and recombinant hGH are used for treating conditions and diseases due to hGH deficiency, e.g., dwarfism in children. Other examples involve interferon, which has known antiviral activity and granulocyte colony stimulating factor, which stimulates the production of white blood cells.

A difficulty in the production of therapeutic peptides, which has limited the use of such agents, lies in engineering an expression system that can be used to express a peptide having a wild-type glycosylation pattern. It is known in the art that improperly or incompletely glycosylated peptides can be immunogenic, leading to neutralization of the peptide and/or the development of an allergic response. Other deficiencies of recombinantly produced glycopeptides include suboptimal potency and rapid clearance from the bloodstream.

One approach of solving the problems inherent in the production of glycosylated peptide therapeutics has been to modify the peptides in vitro after their expression. Post-expression in vitro modification has been used for both modification of glycan structures and introduction of glycans at novel sites. A comprehensive selection of recombinant eukaryotic glycosyltransferases has become available, making in vitro enzymatic synthesis of mammalian glycoconjugates with custom designed glycosylation patterns and glycosyl structures possible. See, for example, U.S. Pat. Nos. 5,876,980; 6,030,815; 5,728,554; 5,922,577; as well as WO/9831826; US2003180835; and WO 03/031464.

In addition to manipulating the structure of glycosyl groups on polypeptides, glycopeptides with one or more non-saccharide modifying groups, such as water soluble polymers, can be prepared. An exemplary polymer that has been conjugated to peptides is poly(ethylene glycol) (“PEG”). PEG has been used to derivatize peptide therapeutics in order to reduce their immunogenicity. For example, U.S. Pat. No. 4,179,337 to Davis et al. discloses non-immunogenic polypeptides such as enzymes and peptide hormones coupled to polyethylene glycol (PEG) or polypropylene glycol (PPG). In addition, PEG-conjugation can be used to reduce the clearance rate of polypeptides from the circulation of a patient, possibly due to increased molecular size of the peptide conjugates.

The principal mode of attachment of PEG, and its derivatives, to peptides is a non-specific bonding through a peptide amino acid residue (see e.g., U.S. Pat. No. 4,088,538 U.S. Pat. No. 4,496,689, U.S. Pat. No. 4,414,147, U.S. Pat. No. 4,055,635, and PCT WO 87/00056). Another mode of attaching PEG to peptides is through the non-specific oxidation of glycosyl residues on a glycopeptide (see e.g., WO 94/05332).

In these non-specific methods, poly(ethyleneglycol) is added in a random, non-specific manner to reactive residues on a peptide backbone. The random addition of PEG molecules has its drawbacks, including a lack of homogeneity of the final product, and the possibility of reduced biological or enzymatic activity of the peptide. Therefore, a derivatization method for therapeutic peptides that results in the formation of a specifically labeled, readily characterizable, essentially homogeneous product is highly desirable.

Specifically, labeled, homogeneous peptide therapeutics can be produced in vitro through the use of enzymes. Unlike the typical non-specific methods for attaching a synthetic polymer or other labels to a peptide, enzyme-based syntheses have the advantages of regioselectivity and stereoselectivity. Two principal classes of enzymes for use in the synthesis of labeled peptides are glycosyltransferases (e.g., sialyltransferases, oligosaccharyltransferases, N-acetylglucosaminyltransferases), and glycosidases. These enzymes can be used for the specific attachment of sugars which can subsequently be altered to comprise a modifying group. Alternatively, glycosyltransferases and modified glycosidases can be used to directly transfer modified sugars to a peptide backbone (see e.g., U.S. Pat. No. 6,399,336, and U.S. Patent Application Publications 20030040037, 20040132640, 20040137557, 20040126838, and 20040142856, each of which are incorporated by reference herein). Methods combining both chemical and enzymatic approaches are also known (see e.g., Yamamoto et al., Carbohydr. Res. 305: 415-422 (1998) and U.S. Patent Application Publication 20040137557, which is incorporated herein by reference).

Carbohydrates are attached to peptides in several ways, of which N-linked to asparagine and mucin-type O-linked to serine and threonine are the most relevant for recombinant glycoprotein therapeutics. Unfortunately, not all polypeptides comprise nitrogen- or oxygen-glycosylation sites as part of their primary amino acid sequence and existing glycosylation sites may not always be suitable for the attachment of a modifying group (e.g., water-soluble or water-insoluble polymers, therapeutic moieties, and or biomolecules). For instance, the glycosylation site may be located within a peptide domain that is not easily accessible for an enzyme, such as a glycosyltransferase. In other cases, attachment of a modified glycosyl residue at an existing glycosylation site may cause an undesirable decrease in biological activity of the polypeptide. Thus, there is a need in the art for methods that permit both the creation of defined glycosylation sites at various positions within the polypeptide sequence and the ability to specifically modify those sites. The current invention addresses these and other needs.

### SUMMARY OF THE INVENTION

It has now been discovered that enzymatic glycosylation and glycoconjugation reactions can be specifically targeted to certain glycosylation sites within a polypeptide. In one embodiment, the present invention provides polypeptide conjugates wherein the amino acid sequence of the polypeptide includes one or more glycosylation consensus sequences, each recognized by an enzyme, such as a glycosyltransferase. In one embodiment, at least one of those glycosylation consensus sequences includes an aromatic amino acid, which is the site of glycosylation.

The glycosylation sites, which are targeted by the glycosylation or glycoconjugation reaction are either present in the wild-type/parent polypeptide or are introduced into the parent or wild-type polypeptide by mutation. Hence, in one embodiment, the polypeptide is a mutant polypeptide including a consensus sequence that does either not exist, or does not exist in the same position, in a wild-type or parent polypeptide corresponding to the mutant polypeptide.

Glycosyl residues and glycosyl mimetic groups that optionally contain a modifying group can be added to an intermediate glycopeptide, either chemically or enzymatically, for instance, via a glycoPEGgylation reaction.

Thus, in a first aspect, the invention provides a polypeptide conjugate including a structure according to Formula (I):

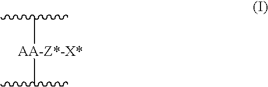

wherein AA is an aromatic amino acid residue, Z* is a member selected from a bond, a glycosyl mimetic moiety and a glycosyl moiety, which is a member selected from mono- and oligosaccharides, and
X* is a member selected from a modifying group, a glycosyl linking group, and a glycosyl linking group that includes a modifying group. In one embodiment, the aromatic amino acid (e.g., tryptophan) is part of a glycosylation consensus sequence of the invention. In a preferred embodiment, the glycosyl linking group includes a modifying group.

In a second aspect, the invention provides a polypeptide conjugate derived from a non-naturally occurring polypeptide. The polypeptide conjugate includes a structure according to Formula (IV):

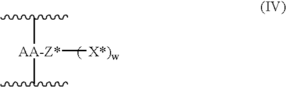

wherein w is an integer selected from 0 to 1, AA is an aromatic amino acid residue, Z* is a member selected from a bond, a glycosyl mimetic moiety and a glycosyl moiety, which is a member selected from mono- and oligosaccharides, and X* is a member selected from a modifying group, a glycosyl linking group and a glycosyl linking group including a modifying group.

In a third aspect, the invention provides a methods for making a polypeptide conjugate of the invention. The method includes: (a) recombinantly producing the polypeptide or non-naturally occurring polypeptide, and (b) enzymatically glycosylating the polypeptide or non-naturally occurring polypeptide at the aromatic amino acid (e.g., tryptophan) residue. The invention further provides methods of using such polypeptide conjugates and their pharmaceutical compositions.

In addition, the invention provides an isolated nucleic acid encoding the non-naturally occurring polypeptides of the invention, as well as an expression cassette and a cell containing the nucleic acid of the invention.

In a fourth aspect, the invention provides a peptide conjugate that includes: (a) a polypeptide; and (b) a modifying group, wherein the modifying group is covalently attached to the polypeptide at a glycosyl or amino acid residue of the polypeptide via a glycosyl linking group, wherein the glycosyl linking group includes at least one thioglycosidic bond.

In a related aspect, the invention provides a method of making a peptide conjugate that includes a thioglycosidic linkage. The method includes: (a) contacting a glycopeptide and a glycosyl linking group and a thioglycoligase under conditions sufficient for the thioglycoligase to form a covalent bond between said glycopeptide and said glycosyl linking group, wherein a member selected from the glycopeptide and the glycosyl linking group includes a sulfhydryl group.

In another aspect, the invention provides a peptide conjugate that includes A polypeptide conjugate comprising: a) a polypeptide; and b) a modifying group, wherein the modifying group is covalently attached to the polypeptide at a glycosyl or amino acid residue of the polypeptide via a glycosyl mimetic linking group, wherein said glycosyl mimetic linking group comprises a structure according to Formula (VII):

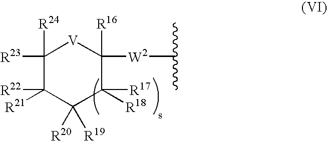

wherein s is an integer from 0 to 3; V and W² are members independently selected from a bond, O, S, NR¹² and CR¹³R¹⁴, wherein R¹², R¹³ and R¹⁴ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, with the proviso that at least one of V and W² is other than O.

R¹⁶, R¹⁷, R¹⁸, R¹⁹, R²⁰, R²¹, R²², R²³ and R²⁴ are members independently selected from H, halogen, CN, OR⁹, SR⁹, NR¹⁰R¹¹, substituted or unsubstituted alkyl, substituted or unsubstituted heteroalkyl, substituted or unsubstituted aryl, substituted or unsubstituted heteroaryl and substituted or unsubstituted heterocycloalkyl, wherein at least two of R¹⁶, R¹⁷, R¹⁸, R¹⁹, R²⁰, R²¹, R²², R²³ and R²⁴, together with the atoms to which they are attached, are optionally joined to form a 5- to 7-membered ring; R⁹ is a member independently selected from H, a metal ion, substituted or unsubstituted alkyl, substituted or unsubstituted heteroalkyl and acyl; R¹⁰ and R¹¹ are members independently selected from H, substituted or unsubstituted alkyl, substituted or unsubstituted heteroalkyl and acyl.

Additional aspects, advantages and objects of the present invention will be apparent from the detailed description that follows.

### BRIEF DESCRIPTION OF THE DRAWINGS

### Detailed Description of the Invention

### I. Abbreviations

PEG, poly(ethyleneglycol); m-PEG, methoxy-poly(ethylene glycol); PPG, poly(propyleneglycol); m-PPG, methoxy-poly(propylene glycol); Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Sia, sialic acid or sialyl; and NeuAc, N-acetylneuraminyl.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, Essentials of Glycobiology Varki et al. eds. CSHL Press (1999).

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

The term “uncharged amino acid” refers to amino acids, that do not include an acidic (e.g., —COOH) or basic (e.g., —NH₂) functional group. Basic amino acids include lysine (K) and arginine (R). Acidic amino acids include aspartic acid (D) and glutamic acid (E).

“Uncharged amino acids include, e.g., glycine (G), valine (V), leucine (L), but also those amino acids that include —OH or —SH groups (e.g., threonine (T), serine (S), cysteine (C).

The term “aromatic amino acid” refers to an amino acid, that includes an aromatic moiety in the amino acid side chain. One example of an aromatic amino acid is tryptophan (W). “Aromatic amino acid” includes natural as well as unnatural amino acids. Unnatural, aromatic amino acids include those that include an indole moiety in their amino acid side chain, wherein the indole ring structure can be substituted with one or more aryl group substituents.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Peptide” refers to a polymer in which the monomers are amino acids and are joined together through amide bonds. Peptides of the present invention can vary in size, e.g., from two amino acids to hundreds or thousands of amino acids, which alternatively is referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are petides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

In the present application, amino acid residues are numbered according to their relative positions from the N-terminal, e.g., the left most residue, which is numbered 1, in a peptide sequence (e.g., a wild-type polypeptide sequence).

The term “non-naturally occurring polypeptide” refers to a form of a polypeptide that includes in its amino acid sequence a glycosylation consensus sequence of the invention. This glycosylation consensus sequence is not present or not present at the same position in the corresponding naturally existing form or any other parent form. A “non-naturally occurring polypeptide” can contain one or more glycosylation consensus sequence of the invention and in addition may include other mutations, e.g., replacements, insertions, deletions, etc.

The term “mutant polypeptide” or “mutein” refers to a form of a peptide that differs from its corresponding parent polypeptide, wild-type form or naturally existing form. A mutant peptide can contain one or more mutations, e.g., replacement, insertion, deletion, etc. which result in the mutant polypeptide.

The term “peptide conjugate,” refers to species of the invention in which a peptide is glycoconjugated to a glycosyl residue. Certain “peptide conjugates” of the invention are glycoconjugated to a modified sugar moiety as set forth herein. In a representative example, the peptide is a mutant peptide including a glycosylation consensus sequence, which is preferably not present in the corresponding parent- or wild-type peptide.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

As used herein, the term “modified sugar,” refers to a naturally- or non-naturally-occurring carbohydrate that is enzymatically added onto an amino acid or a glycosyl residue of a peptide in a process of the invention. The modified sugar is selected from a number of enzyme substrates including, but not limited to sugar nucleotides (mono-, di-, and tri-phosphates), activated sugars (e.g., glycosyl halides, glycosyl mesylates) and sugars that are neither activated nor nucleotides. The “modified sugar” is covalently functionalized with a “modifying group.” Useful modifying groups include, but are not limited to, water-soluble polymers, therapeutic moieties, diagnostic moieties, biomolecules and the like. The modifying group is preferably not a naturally occurring, or an unmodified carbohydrate. The locus of functionalization with the modifying group is selected such that it does not prevent the “modified sugar” from being added enzymatically to a peptide. “Modified sugar” also refers to any glycosyl mimetic group that is functionalized with a modifying group and which is a substrate for a natural or modified enzyme, such as a glycosyltransferase of the invention.

The term “glycosyl-mimetic moiety,” as used herein refers to a moiety, which structurally resembles a glycosyl moiety (e.g., a hexose or a pentose). Examples of “glycosyl-mimetic moiety” include those moieties, wherein the glycosidic oxygen or the ring oxygen of a glycosyl moiety, or both, has been replaced with a bond or another atom (e.g., sufur), or another moiety, such as a carbon- (e.g., CH₂), or nitrogen-containing group (e.g., NH). Examples include substituted or unsubstituted cyclohexyl derivatives, cyclic thioethers, cyclic secondary amines as well as moieties including a thioglycosidic bond, and the like. Other examples of “glycosyl-mimetic moiety” include ring structures with double bonds as well as ring structures, wherein one of the ring carbon atoms carries a carbonyl group or another double-bonded substituent, such as a hydrazone. In one example, the “glycosyl-mimetic moiety” is transferred in an enzymatically catalyzed reaction onto an amino acid residue of a polypeptide or a glycosyl moiety of a glycopeptide. This can, for instance, be accomplished by activating the “glycosyl-mimetic moiety” with a leaving group, such as a halogen.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine). An exemplary polysaccharide is poly(sialic acid). An exemplary poly(ether) is poly(ethylene glycol), e.g., m-PEG. Poly(ethylene imine) is an exemplary polyamine, and poly(acrylic) acid is a representative poly(carboxylic acid).

The polymer backbone of the water-soluble polymer can be poly(ethylene glycol) (i.e. PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, difunctional PEG, multiarmed PEG, forked PEG, branched PEG, pendent PEG (i.e. PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)ₘ in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as the polymer backbone.

Many other polymers are also suitable for the invention. Polymer backbones that are non-peptidic and water-soluble, with from 2 to about 300 termini, are particularly useful in the invention. Examples of suitable polymers include, but are not limited to, other poly(alkylene glycols), such as poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, as well as copolymers, terpolymers, and mixtures thereof. Although the molecular weight of each chain of the polymer backbone can vary, it is typically in the range of from about 100 Da to about 100,000 Da, often from about 6,000 Da to about 80,000 Da.

The term “glycoconjugation,” as used herein, refers to the enzymatically mediated conjugation of a modified sugar species to an amino acid or glycosyl residue of a polypeptide, e.g., a mutant human growth hormone of the present invention. In one example, the modified sugar is covalently attached to one or more modifying groups. A subgenus of “glycoconjugation” is “glycol-PEGylation” or “glyco-PEGylation”, in which the modifying group of the modified sugar is poly(ethylene glycol) or a derivative thereof, such as an alkyl derivative (e.g., m-PEG) or a derivative with a reactive functional group (e.g., H₂N-PEG, HOOC-PEG).

The terms “large-scale” and “industrial-scale” are used interchangeably and refer to a reaction cycle that produces at least about 250 mg, preferably at least about 500 mg, and more preferably at least about 1 gram of glycoconjugate at the completion of a single reaction cycle.

The term, “glycosyl linking group,” as used herein refers to a glycosyl residue to which a modifying group (e.g., PEG moiety, therapeutic moiety, biomolecule) is covalently attached; the glycosyl linking group joins the modifying group to the remainder of the conjugate. In the methods of the invention, the “glycosyl linking group” becomes covalently attached to a glycosylated or unglycosylated peptide, thereby linking an agent (e.g., a modifying group) to an amino acid and/or glycosyl residue on the peptide. A “glycosyl linking group” is generally derived from a “modified sugar” by the enzymatic attachment of the “modified sugar” to an amino acid and/or glycosyl residue of the peptide. The glycosyl linking group can be a saccharide-derived structure that is modified (e.g. degraded) prior to and/or during formation of the modifying group-modified sugar cassette (e.g., oxidations Schiff base formations reduction), or the glycosyl linking group may be intact. An “intact glycosyl linking group” refers to a linking group that is derived from a glycosyl moiety in which the saccharide monomer that links the modifying group and to the remainder of the conjugate is not degraded, e.g., oxidized, e.g., by sodium metaperiodate. “Intact glycosyl linking groups” of the invention may be derived from a naturally occurring oligosaccharide by addition of glycosyl unit(s) or removal of one or more glycosyl unit from a parent saccharide structure. The term, “glycosyl linking group” includes “glycosyl-mimetic linking group”, in which a glycosyl moiety is replaced with a “glycosyl-mimetic moiety”.

The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO and the like.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is bound to a carrier, e.g, multivalent agents. Therapeutic moiety also includes proteins and constructs that include proteins. Exemplary proteins include, but are not limited to, Erythropoietin (EPO), Granulocyte Colony Stimulating Factor (GCSF), Granulocyte Macrophage Colony Stimulating Factor (GMCSF), Interferon (e.g., Interferon-α, -β, -γ), Interleukin (e.g., Interleukin II), serum proteins (e.g., Factors VII, VIIa, VIII, IX, and X), Human Chorionic Gonadotropin (HCG), Follicle Stimulating Hormone (FSH) and Lutenizing Hormone (LH) and antibody fusion proteins (e.g. Tumor Necrosis Factor Receptor ((TNFR)/Fc domain fusion protein)).

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of peptides with anti-tumor activity, e.g. TNF-α. Conjugates include, but are not limited to those formed between a therapeutic protein and a glycoprotein of the invention. A representative conjugate is that formed between PSGL-1 and TNF-α.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diptheria toxin, and snake venom (e.g., cobra venom).

As used herein, “a radioactive agent” includes any radioisotope that is effective in diagnosing or destroying a tumor. Examples include, but are not limited to, indium-111, cobalt-60. Additionally, naturally occurring radioactive elements such as uranium, radium, and thorium, which typically represent mixtures of radioisotopes, are suitable examples of a radioactive agent. The metal ions are typically chelated with an organic chelating moiety.

Many useful chelating groups, crown ethers, cryptands and the like are known in the art and can be incorporated into the compounds of the invention (e.g., EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

The term “therapy” refers to “treating” or “treatment” of a disease or condition including preventing the disease or condition from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any grammatically equivalent term means the amount that, when administered to an animal or human for treating a disease, is sufficient to effect treatment for that disease.

The term “isolated” refers to a material that is substantially or essentially free from components, which are used to produce the material. For peptide conjugates of the invention, the term “isolated” refers to material that is substantially or essentially free from components, which normally accompany the material in the mixture used to prepare the peptide conjugate. “Isolated” and “pure” are used interchangeably. Typically, isolated peptide conjugates of the invention have a level of purity preferably expressed as a range. The lower end of the range of purity for the peptide conjugates is about 60%, about 70% or about 80% and the upper end of the range of purity is about 70%, about 80%, about 90% or more than about 90%.

When the peptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

“Essentially each member of the population,” as used herein, describes a characteristic of a population of peptide conjugates of the invention in which a selected percentage of the modified sugars added to a peptide are added to multiple, identical acceptor sites on the peptide. “Essentially each member of the population” speaks to the “homogeneity” of the sites on the peptide conjugated to a modified sugar and refers to conjugates of the invention, which are at least about 80%, preferably at least about 90% and more preferably at least about 95% homogenous.

“Homogeneity,” refers to the structural consistency across a population of acceptor moieties to which the modified sugars are conjugated. Thus, in a peptide conjugate of the invention in which each modified sugar moiety is conjugated to an acceptor site having the same structure as the acceptor site to which every other modified sugar is conjugated, the peptide conjugate is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The purity of the peptide conjugates is typically determined by one or more methods known to those of skill in the art, e.g., liquid chromatography-mass spectrometry (LC-MS), matrix assisted laser desorption mass time of flight spectrometry (MALDITOF), capillary electrophoresis, and the like.

“Substantially uniform glycoform” or a “substantially uniform glycosylation pattern,” when referring to a glycopeptide species, refers to the percentage of acceptor moieties that are glycosylated by the glycosyltransferase of interest (e.g., fucosyltransferase). For example, in the case of a α1,2 fucosyltransferase, a substantially uniform fucosylation pattern exists if substantially all (as defined below) of the Galβ1,4-GlcNAc-R and sialylated analogues thereof are fucosylated in a peptide conjugate of the invention. It will be understood by one of skill in the art, that the starting material may contain glycosylated acceptor moieties (e.g., fucosylated Galβ1,4-GlcNAc-R moieties). Thus, the calculated percent glycosylation will include acceptor moieties that are glycosylated by the methods of the invention, as well as those acceptor moieties already glycosylated in the starting material.

The term “substantially” in the above definitions of “substantially uniform” generally means at least about 40%, at least about 70%, at least about 80%, or more preferably at least about 90%, and still more preferably at least about 95% of the acceptor moieties for a particular glycosyltransferase are glycosylated.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR, ═O, ═NR′, ═N—OR, —NR′R″, —SR, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″′)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″′)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si) and boron (B).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocycloalkyl groups.

The phrase “therapeutically effective amount” as used herein means that amount of a compound, material, or composition comprising a compound of the present invention which is effective for producing some desired therapeutic effect by inhibition of DAAO in at least a sub-population of cells in an animal and thereby blocking the biological consequences of that pathway in the treated cells, at a reasonable benefit/risk ratio applicable to any medical treatment.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The graphic representations of racemic, ambiscalemic and scalemic or enantiomerically pure compounds used herein are taken from Maehr, J. Chem. Ed., 62: 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

The terms “enantiomeric excess” and diastereomeric excess” are used interchangeably herein. Compounds with a single stereocenter are referred to as being present in “enantiomeric excess,” those with at least two stereocenters are referred to as being present in “diastereomeric excess.”

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds. ORGANIC FUNCTIONAL GROUP PREPARATIONS, Academic Press, San Diego, 1989).

“Non-covalent protein binding groups” are moieties that interact with an intact or denatured polypeptide in an associative manner. The interaction may be either reversible or irreversible in a biological milieu. The incorporation of a “non-covalent protein binding group” into a chelating agent or complex of the invention provides the agent or complex with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, thiophosphate, phosphonate, carboxylate, boronate, sulfate, sulfone, sulfonate, thiosulfate, and thiosulfonate.

### III. Introduction

### Carbon- and Nitrogen-Linked Glycosylation

In one embodiment, the present invention provides polypeptide conjugates wherein the amino acid sequence of the polypeptide contains one or more glycosylation consensus sequences, wherein each consensus sequence is recognized by an enzyme, such as a glycosyltransferase (e.g., carbon-mannosyl-transferase or nitrogen-mannosyl-transferase). In one example, the enzyme catalyses the transfer of a glycosyl moiety from a glycosyl donor molecule to a carbon or nitrogen atom of an amino acid side chain, wherein the amino acid (e.g., tryptophan), which is the site of glycosylation, is part of a glycosylation consensus sequence. Exemplary glycosyl residues that can be conjugated to the glycosylation site include mannose (Man), N-acetylgalactosamine (GalNAc), galactose (Gal), N-acetylglucosamine (GlcNAc), glucose (Glc), fucose (Fuc) and xylose (Xyl) as well as combinations and derivatives and glycosyl-mimetic moieties thereof.

The invention also provides glycopeptide conjugates, in which a modified sugar moiety is attached either directly or indirectly (e.g., via a glycosyl residue) to a carbon- or nitrogen-glycosylation site of the invention located within the polypeptide. Also provided are methods for producing the glycopeptide conjugates of the invention.

The glycosylation and glycomodification (e.g., glycoPEGylation) methods of the invention can be practiced on any peptide that has incorporated into its amino acid sequence a glycosylation consensus sequence of the invention (e.g., a carbon- or nitrogen-glycosylation consensus sequence). The methods are especially useful for the preparation of carbon- and nitrogen-linked glycoconjugates of polypeptides, in which the glycosylation consensus sequence has been introduced into the amino acid sequence of a “parent peptide” by mutation. The parent peptide can be any peptide. Exemplary parent peptides include wild-type peptides as well as peptides, which are additionally modified from their naturally occurring counterpart (e.g., by previous mutation). In one embodiment, the parent peptide is preferably a peptide used as a pharmaceutical agent, such as human growth hormone (hGH), erythropoietin (EPO) or a therapeutic monoclonal antibody. Accordingly, the present invention provides glycoconjugates of pharmaceutical peptides that include within their amino acid sequence one or more glycosylation consensus sequence of the invention.

Additional methods include the elaboration, trimming back and/or modification of the carbon- and/or nitrogen-linked glycosyl residues.

### Sulfur-Linked Glycosylation

The invention further provides peptide conjugates including a thioglycosidic bond. In one example, the peptide conjugate includes a glycosyl linking group, wherein the glycosyl linking group is covalently attached to the remainder of the peptide conjugate via a thioglycosidic linkage. Thus, the modifying group is covalently attached to the peptide or glycopeptide via a glycosyl linking group that contains a thioglycosidic bond.

In addition, the invention provides methods of making the described sulfur-linked peptide conjugates. The methods utilize a thioglycoligase as well as one or more glycosyl residues that contain a sulfhydryl group. Exemplary conjugates are formed by contacting an activated donor glycoside and a deoxythio sugar in the presence of a thioglycoligase.

### IV. Compositions

### Peptide Conjugates

In one embodiment, the present invention provides a polypeptide conjugate between a polypeptide and a selected modifying group, in which the modifying group is covalently bound to the peptide through a glycosyl linking group (e.g., an intact glycosyl linking group). The glycosyl linking group is bound either directly to an amino acid residue within a glycosylation consensus sequence or, alternatively, it is bound to the peptide through one or more additional glycosyl residues. Methods of preparing the conjugates are set forth herein and in U.S. Pat. Nos. 5,876,980; 6,030,815; 5,728,554; 5,922,577; WO 98/31826; US2003180835; and WO 03/031464, which are incorporated herein by reference.

Hence, in one example, the invention provides a polypeptide conjugate including a structure according to Formula (I):

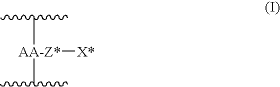

wherein AA is an aromatic amino acid residue, Z* is a member selected from a bond and a glycosyl moiety, which is a member selected from mono- and oligosaccharides, and X* is a member selected from a modifying group, a glycosyl linking group, and a glycosyl linking group that includes a modifying group. In one embodiment, the aromatic amino acid (e.g., tryptophan) is part of a glycosylation consensus sequence of the invention. In a preferred embodiment, the glycosyl linking group includes a modifying group.

In another embodiment, the invention provides a polypeptide conjugate wherein the polypeptide is a mutant polypeptide and wherein the mutant polypeptide conjugate includes a structure according to Formula (IV):

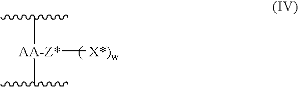

wherein w is an integer selected from 0 to 1, AA is an aromatic amino acid residue, Z* is a member selected from a bond and a glycosyl moiety, which is a member selected from mono- and oligosaccharides, and X* is a member selected from a modifying group, a glycosyl linking group and a glycosyl linking group including a modifying group.

In an exemplary embodiment, Z* in Formula (I) and Formula (IV) includes a member selected from Man, Gal, GalNAc, GlcNAc, Xyl, Glc, Sia and combinations thereof. In a preferred embodiment, Z* includes a mannose moiety.

The conjugates of the invention will typically correspond to the general structure:

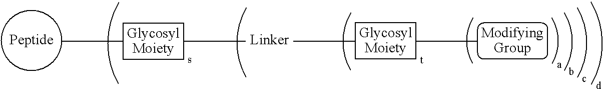

in which the symbols a, b, c, d and s represent a positive, non-zero integer; and t is either 0 or a positive integer. The “modifying group” is a therapeutic agent, a bioactive agent, a detectable label, a water-soluble moiety or the like. The linker can be any of a wide array of linking groups, infra. Alternatively, the linker may be a single bond. The identity of the peptide is without limitation.

As discussed herein, the modifying group is essentially any species that can be attached to a saccharide unit, resulting in a “modified sugar” that is recognized by an appropriate transferase enzyme, which appends the modified sugar onto the peptide or glycopeptide. Exemplary modifying groups are selected from glycosidic and non-glycosidic modifying groups and include polymers (e.g., PEG) and peptides, such as enzymes, antibodies, antigens, etc. Exemplary non-glycosidic modifying groups are selected from linear and branched and can include one or more independently selected polymeric moieties, such as poly(ethylene glycol) and derivatives thereof. Additional modifying groups are described herein below. In one embodiment, the glycosyl linking group includes at least one modifying group.

In an exemplary embodiment, the modifying group is a water-soluble polymeric group, e.g., PEG, m-PEG, PPG, m-PPG, etc. The water-soluble polymer is covalently attached to the peptide via a glycosyl linking group. The glycosyl linking group is covalently attached to either an amino acid residue or a glycosyl residue of the peptide. Alternatively, the glycosyl linking group is attached to one or more glycosyl units of a glycopeptide. The invention also provides conjugates in which the glycosyl linking group (e.g., Sia or Man) is attached directly to an amino acid residue (e.g., tryptophan).

In addition to providing conjugates that are formed through an enzymatically added glycosyl linking group, the present invention provides conjugates that are highly homogenous in their substitution patterns. Using the methods of the invention, it is possible to form peptide conjugates in which essentially all of the modified sugar moieties across a population of conjugates of the invention are attached to a structurally identical amino acid or glycosyl residue. Thus, in one embodiment, the invention provides a peptide conjugate having a population of water-soluble polymeric moieties, which are covalently bound to the peptide through a glycosyl linking group. In another conjugate of the invention, essentially each member of the population is bound via the glycosyl linking group to a glycosyl residue of the peptide, and each glycosyl residue of the peptide to which the glycosyl linking group is attached has the same structure.

In another embodiment, essentially every member of the population of water soluble polymer moieties is bound to an amino acid residue of the peptide via a glycosyl linking group, and each amino acid residue having an intact glycosyl linking group attached thereto has the same structure.

Exemplary peptide conjugates of the invention include a tryptophan-C²-linked mannose residue that is bound to the glycosylation site through the action of a carbon-mannosyltransferase. Other exemplary peptide conjugates include a tryptophan N¹-linked mannose residue, which is attached to the polypeptide using a nitrogen-mannosyltransferase. The mannose (Man) itself may be the glycosyl linking group. In another example, the mannose is further elaborated by another glycosyl residue, such as a Gal, GalNAc, GlcNAc or Sia residue, either of which can act as the glycosyl linking group. In representative embodiments, the C-linked saccharyl residue (-Z*-X*) includes a member selected from Man-X*, Man-GlcNAc-X*, Man-GlcNAc-Gal-X*, Man-GlcNAc-Gal-Gal-X*, Man-GalNAc-X*, Man-GalNAc-Gal-X* and Man-GalNAc-Gal-Gal-X*, in which X* is a modifying group or a glycosyl linking group.

### Polypeptides

The polypeptide conjugates of the invention can include any polypeptide, such as wild-type and mutant polypeptides. In one embodiment, the invention describes isolated, mutant polypeptides having an amino acid sequence that includes one or more glycosylation consensus sequences of the invention, which are each recognized by an enzyme, such as a glycosyltransferase. In one example, the mutant polypeptides provided by the present invention include an amino acid sequence that is recognized as a mannose acceptor by one or more wild-type or mutant C-mannosyl- or N-mannosyltransferases.

Thus, in one embodiment, the invention provides a mutant polypeptide including a mutant amino acid sequence, wherein the mutant amino acid sequence includes a glycosylation consensus sequence that contains an aromatic amino acid (e.g., tryptophan), wherein the aromatic amino acid is the site of glycosylation.

Exemplary mutant polypeptides include at least one glycosylation consensus sequence that is either not present does not exist in the same position in the corresponding parent or wild-type polypeptide. The amino acid sequence of the mutant polypeptide may contain a combination of naturally occurring and unnatural glycosylation sites.

Exemplary parent and wild-type polypeptides, which can optionally be modified to incorporate one or more glycosylation consensus sequences of the invention include: bone morphogenetic protein (e.g., BMP-2, BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (ATT, or α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), TNF receptor-IgG Fc region fusion protein (Enbrel™), anti-HER2 monoclonal antibody (Herceptin™), monoclonal antibody to protein F of respiratory syncytial virus (Synagis™), monoclonal antibody to TNF-α (Remicade™), monoclonal antibody to glycoprotein IIb/IIIa (Reopro™), monoclonal antibody to CD20 (Rituxan™), anti-thrombin III (AT III), human chorionic gonadotropin (hCG), alpha-galactosidase (Fabrazyme™), alpha-iduronidase (Aldurazyme™), follicle stimulating hormone (FSH), beta-glucosidase, anti-TNF-alpha monoclonal antibody (MLB 5075), glucagon-like peptide-1 (GLP-1), beta-glucosidase (MLB 5064), alpha-galactosidase A (MLB 5082), fibroblast growth factor (FGF), Factor VII, Factor VIII, Factor IX, prokinetisin and extendin-4, as well as any modified versions (e.g., mutants) of any of the above listed polypeptides.

The mutant polypeptides of the invention can be generated using methods known in the art as well as those described herein below.

### Glycosylation Consensus Sequences

Exemplary glycosylation consensus sequences of the invention are short (e.g., 2 to 15) amino acid sequences that contain at least one amino acid with an aromatic amino acid side chain (aromatic amino acid). The amino acid can be a natural (e.g., tryptophan, tyrosine, phenylalanine) or an unnatural amino acid, such as those wherein the amino acid side chain includes an indole moiety (e.g., a tryptophan derivative).

### Carbon-Glycosylation

In one embodiment, the polypeptide or mutant polypeptide of the invention incorporates a glycosylation consensus sequence that includes an amino acid sequence, which is a member selected from:

| | |
|---|---|
| WX¹(X²W)ₘ; | |
| WX¹X²WX³(X⁴W)ₙ; | |
| WX¹X²C; | |
| WX¹X²WX³X⁴C; | |
| WX¹X²WX³X⁴WX⁵X⁶C; | (SEQ ID NO: 1) |
| and | |
| AX¹X²WX³X⁴X⁵X⁶X⁷C | |

wherein m and n are integers from 0-1, W is tryptophan, C is cysteine and wherein each of X¹, X², X³, X⁴, X⁵, X⁶ and X⁷ is a member independently selected from natural and unnatural amino acids.

In an exemplary embodiment, X¹, X², X³, X⁴, X⁵, X⁶ and X⁷ are members independently selected from glutamic acid (E), glutamine (Q), aspartic acid (D), asparagine (N), threonine (T), serine (S) and uncharged amino acids. In one embodiment, X¹, X³ and X⁵ are members independently selected from serine (S), threonine (T) and uncharged amino acids and are preferably not selected from large hydrophobic amino acids.

Exemplary carbon-glycosylation consensus sequences of the invention include:

| | |
|---|---|
| WSX²W | (SEQ ID NO: 2) |
| WTX²W | (SEQ ID NO: 3) |
| WAX²W; | (SEQ ID NO: 4) |
| for example: WAQW | (SEQ ID NO: 5) |
| WSX²WS; | (SEQ ID NO: 6) |
| for example WSQWS | (SEQ ID NO: 7) |
| WSX²C | (SEQ ID NO: 8) |
| WTX²C | (SEQ ID NO: 9) |
| WSCWSSW | (SEQ ID NO: 10) |
| WGCWSSW | (SEQ ID NO: 11) |

wherein X² is as defined above.

In another embodiment, the above sequences are optionally preceded and/or followed by one or more additional amino acids to extended consensus sequences of the invention. In an exemplary embodiment, sequences are selected from:

| | |
|---|---|
| (Y1)ₚWX¹(X²W)ₘ(Y2)_{q}; | |
| (Y1)ₚWX¹X²WX³(X⁴W)ₙ(Y2)_{q}; | |
| (Y1)ₚWX¹X²C(Y2)_{q}; | |
| (Y1)ₚWX¹X²WX³X⁴C(Y2)_{q}; | |
| (Y1)ₚWX¹X²WX³X⁴WX⁵X⁶C(Y2)_{q}; | (SEQ ID NO: 12) |
| and | |
| (Y1)ₚWX¹X²WX³X⁴X⁵X⁶X⁷C(Y2)_{q} | |

wherein the integers p and q are independently selected from 0 and 1. Y¹ and Y² are members independently selected from natural and unnatural amino acids. In an exemplary embodiment, Y¹ and Y² are members selected from uncharged amino acids, preferably those amino acids with a non-bulky side chain, such as glycine and alanine.

In one embodiment, the above described glycosylation consensus sequences are recognized by a wild-type or mutant glycosyltransferase. In a preferred embodiment, the glycosyltransferase is recombinantly produced. In one example, the glycosyltransferase is a mannosyltransferase. In another example, the mannosyltransferase is a C-mannosyltransferase, which can be used to transfer a glycosyl (e.g., mannosyl) residue to the C²-position of a tryptophan side chain located within a glycosylation consensus sequence of the invention as outlined in Scheme 1. Hence, the current invention provides polypeptide conjugates wherein a tryptophan residue is glycosylated at the C²-position of the indole moiety. In a preferred embodiment, the glycosyl residue, which is attached to the indole moiety, is mannose or a modified mannose moiety.

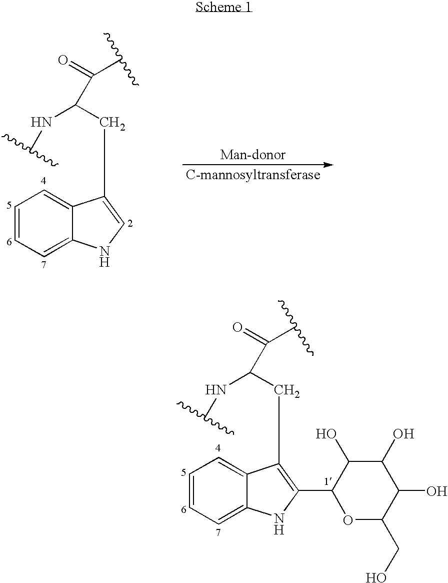

### Nitrogen-Glycosylation

In another example, the mannosyltransferase is a nitrogen-mannosyltransferase, which can be used to transfer a glycosyl (e.g., mannosyl) residue to the N¹-position of a tryptophan side chain located within a glycosylation consensus sequence of the invention as outlined in Scheme 2.

Thus, the current invention also provides polypeptide conjugates wherein a tryptophan residue is glycosylated at the N¹-position of the indole moiety. In a preferred embodiment, the glycosyl residue, which is attached to the indole moiety, is mannose or a modified mannose moiety.

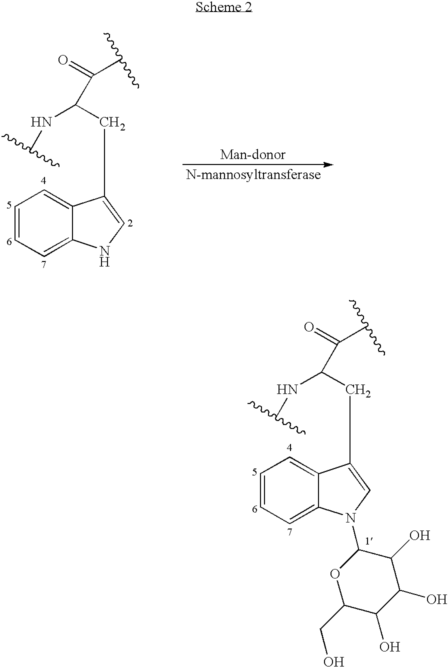

### Non-Naturally Occurring Polypeptides

The glycosylation consensus sequences of the invention can be introduced into the amino acid sequence of any parent peptide. In one embodiment, the parent sequence is mutated in such a way that the glycosylation site is inserted into the parent sequence by adding the entire length and respective number of amino acids to the amino acid sequence of the parent peptide. In another embodiment, the mutant glycosylation site replaces one or more amino acids of the parent peptide. In a further embodiment, the mutation is introduced into the parent peptide, using one or more of the pre-existing amino acids to become part of the consensus sequence. For instance, a tryptophan residue in a wild-type peptide is maintained and those amino acids immediately following the tryptophan are mutated to create an exemplary glycosylation consensus sequence.

The glycosylation consensus sequences of the invention can be introduced into a parent peptide at any position within its amino acid sequence. In one example, the consensus sequence is located at or near the amino-terminus of the parent peptide (amino-terminal mutants). In another example, the consensus sequence is located at or near the C-terminus of the parent peptide (carboxy-terminal mutants). In yet another example, the consensus sequence is located anywhere between the N-terminus and the C-terminus of the parent peptide (internal mutants).

An exemplary parent polypeptide is recombinant human BMP-7, which has the following amino acid sequence (140 amino acids):

(SEQ ID NO: 13)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCH

In one exemplary embodiment, mutations are introduced into the wild-type BMP-7 amino acid sequence (SEQ ID NO: 13) replacing the corresponding number of amino acids in the parent sequence, resulting in a mutant peptide that contains the same number of amino acid residues as the parent peptide. For instance, directly substituting four amino acids normally in BMP-7 with the consensus sequence tryptophan-threonine-glutamine-tryptophan (WTQW) and then sequentially moving the WTQW sequence towards the C-terminus of the peptide provides 136 BMP-7 peptide analogs containing WTQW. Exemplary sequences according to this embodiment are listed below.

Exemplary BMP-7 Mutants Wherein Four Amino Acids are Replaced with WTQW (140 Amino Acids):

(SEQ ID NO: 14)

M¹WTQWKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCH

(SEQ ID NO: 15)

M¹SWTQWQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCH

(SEQ ID NO: 16)

M¹STWTQWRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCH

And so forth, until:

(SEQ ID NO: 17)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRAWTQW

In another exemplary embodiment, mutations are introduced into the wild-type BMP-7 amino acid sequence (SEQ ID NO: 13) by adding one or more amino acids to the parent sequence. For instance, the glycosylation sequence WTQW is added to the parent BMP-7 sequence to replace 3, 2, 1 or none of the existing amino acids. In one example, the consensus sequence is added to either the N- or C-terminus of the parent sequence. Exemplary sequences according to this embodiment are listed below.

### Exemplary BMP-7 Mutants Including WTQW (141 to 144 Amino Acids):

(SEQ ID NO: 18)

M¹WTQWSTGSKQRSQNRSKTPKNQEALRMANVAENSSSDQRQACKKHELY
VSFRDLGWQDWIIAPEGYAAYYCEGECAFPLNSYMNATNHAIVQTLVHFI
NPETVPKPCCAPTQLNAISVLYFDDSSNVILKKYRNMVVRACGCH

(SEQ ID NO: 19)

M¹WTQWTGSKQRSQNRSKTPKNQEALRMANVAENSSSDQRQACKKHELYV
SFRDLGWQDWIIAPEGYAAYYCEGECAFPLNSYMNATNHAIVQTLVHFIN
PETVPKPCCAPTQLNAISVLYFDDSSNVILKKYRNMVVRACGCH

(SEQ ID NO: 20)

M¹WTQWGSKQRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

(SEQ ID NO: 21)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGWTQW

(SEQ ID NO: 22)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCWTQW

(SEQ ID NO: 23)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCHWTQW

In another example, a glycosylation consensus sequence is added to the peptide sequence at any amino acid position, thereby adding one or more amino acids to the parent sequence. In this example, the maximum number of added amino acid residues corresponds to the length of the inserted glycosylation site. In an exemplary embodiment, the parent sequence is extended by exactly two amino acids. For example, the consensus sequence WTQW is added to the parent BMP-7 peptide replacing 2 amino acids normally present in BMP-7. Exemplary sequences according to this embodiment are listed below.

### Exemplary BMP-7 Mutants Including WTQW (142 Amino Acids):

(SEQ ID NO: 24)

M¹WTQWGSKQRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

(SEQ ID NO: 25)

M¹SWTQWSKQRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

(SEQ ID NO: 26)

M¹STWTQWKQRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

(SEQ ID NO: 27)

M¹STGWTQWQRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

(SEQ ID NO: 28)

M¹STGSWTQWRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

An analogous example involves the addition of the WTQW consensus sequence to the parent BMP-7 peptide replacing 1 amino acid normally present in BMP-7 (triple amino acid insertion).

Yet another example involves the addition of the consensus sequence to the parent BMP-7 peptide replacing none of the amino acids normally present in BMP-7 adding the entire length of the consensus sequence (insertion of four amino acids) to any position within the parent peptide. Exemplary sequences according to this embodiment are listed below.

### Exemplary BMP-7 Mutants Including WTQW (144 Amino Acids):

(SEQ ID NO: 29)

M¹SWTQWTGSKQRSQNRSKTPKNQEALRMANVAENSSSDQRQACKKHELY
VSFRDLGWQDWIIAPEGYAAYYCEGECAFPLNSYMNATNHAIVQTLVHFI
NPETVPKPCCAPTQLNAISVLYFDDSSNVILKKYRNMVVRACGCH

(SEQ ID NO: 30)

M¹STWTQWGSKQRSQNRSKTPKNQEALRMANVAENSSSDQRQACKKHELY
VSFRDLGWQDWIIAPEGYAAYYCEGECAFPLNSYMNATNHAIVQTLVHFI
NPETVPKPCCAPTQLNAISVLYFDDSSNVILKKYRNMVVRACGCH

(SEQ ID NO: 31)

M¹STGWTQWSKQRSQNRSKTPKNQEALRMANVAENSSSDQRQACKKHELY
VSFRDLGWQDWIIAPEGYAAYYCEGECAFPLNSYMNATNHAIVQTLVHFI
NPETVPKPCCAPTQLNAISVLYFDDSSNVILKKYRNMVVRACGCH

Similar iterations of BMP-7 mutants can be generated using any of the oxygen glycosylation sites of the invention. For instance, instead of WTQW the sequence WSQWS (SEQ ID NO: 32) can be used. In an exemplary embodiment WSQWS is introduced into the parent peptide replacing 5 amino acids normally present in BMP-7.

In another example the oxygen glycosylation site WSQWS (SEQ ID NO: 32) is added to the wild-type BMP-7 sequence at either the N- or C-terminal area of the parent sequence, adding 1 to 5 amino acids to the wild-type. Exemplary sequences according to this embodiment are listed below.

### Exemplary BMP-7 Mutants Including WSQWS (141-145 Amino Acids)

Amino-terminal mutants:

(SEQ ID NO: 33)

M¹WSQWSSTGSKQRSQNRSKTPKNQEALRMANVAENSSSDQRQACKKHEL
YVSFRDLGWQDWIIAPEGYAAYYCEGECAFPLNSYMNATNHAIVQTLVHF
INPETVPKPCCAPTQLNAISVLYFDDSSNVILKKYRNMVVRACGCH

(SEQ ID NO: 34)

M¹WSQWSTGSKQRSQNRSKTPKNQEALRMANVAENSSSDQRQACKKHELY
VSFRDLGWQDWIIAPEGYAAYYCEGECAFPLNSYMNATNHAIVQTLVHFI
NPETVPKPCCAPTQLNAISVLYFDDSSNVILKKYRNMVVRACGCH

(SEQ ID NO: 35)

M¹WSQWSGSKQRSQNRSKTPKNQEALRMANVAENSSSDQRQACKKHELYV
SFRDLGWQDWIIAPEGYAAYYCEGECAFPLNSYMNATNHAIVQTLVHFIN
PETVPKPCCAPTQLNAISVLYFDDSSNVILKKYRNMVVRACGCH

(SEQ ID NO: 36)

M¹WSQWSSKQRSQNRSKTPKNQEALRMANVAENSSSDQRQACKKHELYVS
FRDLGWQDWIIAPEGYAAYYCEGECAFPLNSYMNATNHAIVQTLVHFINP
ETVPKPCCAPTQLNAISVLYFDDSSNVILKKYRNMVVRACGCH

(SEQ ID NO: 37)

M¹WSQWSKQRSQNRSKTPKNQEALRMANVAENSSSDQRQACKKHELYVSF
RDLGWQDWIIAPEGYAAYYCEGECAFPLNSYMNATNHAIVQTLVHFINPE
TVPKPCCAPTQLNAISVLYFDDSSNVILKKYRNMVVRACGCH
Carboxy-terminal mutants

(SEQ ID NO: 38)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCHWSQWS

(SEQ ID NO: 39)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGCWSQWS

(SEQ ID NO: 40)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACGWSQWS

(SEQ ID NO: 41)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRACWSQWS

(SEQ ID NO: 42)

M¹STGSKQRSQNRSKTPKNQEALRMANVAENSSSDQRQACKKHELYVSFR
DLGWQDWIIAPEGYAAYYCEGECAFPLNSYMNATNHAIVQTLVHFINPET
VPKPCCAPTQLNAISVLYFDDSSNVILKKYRNMVVRAWSQWS

The substitution of wild-type amino acids with glycosylation consensus sequences as well as the insertion of those sequences without substitution can be performed at pre-selected, specific regions of the parent protein. Mutations that cause little or no disruption of the tertiary structure of the peptide (compared with the tertiary structure of the parent or wild-type peptide) are generally preferred. In nature, glycosylation of the peptide backbone usually occurs in protein loops and not within helical or beta-sheet structures. For instance, a tryptophan side chain that is to be glycosylated must be accessible to the glycosyltransferase (e.g., C-mannosyltransferase). Hence, a conformation, in which the tryptophan side side chain is not oriented inward, forming hydrogen bonds with other regions of the peptide or neighboring proteins, is best suited to enable efficient glycosylation. Therefore, taking the tertiary structure of the wild-type or parent peptide into consideration, will allow for pre-selection of promising mutation sites.

Ideally, the crystal structure of a protein can be used to identify those domains of a peptide that are most suitable (e.g., easily accessible) for mutation and introduction of a glycosylation site. For those peptides, for which crystal structures are not available, the amino acid sequence can be used to pre-select promising mutation sites (e.g., prediction of loop areas versus beta-sheet conformations).

For example, the crystal structure of the protein BMP-7 contains two extended loop regions between A⁷² and A⁸⁶ as well as I⁹⁶ and P¹⁰³. Generating BMP-7 mutants, in which the glycosylation site is placed within those regions of the peptide sequence, may result in peptides, wherein the mutation causes little or no disruption of the original tertiary structure of the peptide. Biologically active BMP-7 mutants of the present invention include any BMP-7 polypeptide, in part or in whole, with one or more mutations that do not result in substantial or entire loss of its biological activity as measured by any suitable functional assay known to one of skill in the art.

In order to identify optimal positions for the oxygen-glycosylation site within the pre-selected sequence areas of the parent peptide, a variety of mutants are created and then screened for desired activity. In an exemplary embodiment, the mutation site is “moved” along the parent peptide from the N-terminus towards the C-terminus (for instance, one amino acid at a time). This procedure has been termed “sequon scanning”.

In an exemplary embodiment, the oxygen glycosylation site WTQW (SEQ ID NO: 43) is placed anywhere into selected peptide regions either by substitution of existing amino acids or by insertion. Exemplary sequences according to this embodiment are listed below.

### Exemplary BMP-7 Mutants Including WTQW Between A⁷³ and A⁸² or I⁹⁵ and P¹⁰³

| Substitution of existing amino acids A⁷³ to A⁸² | |
|---|---|
| ---A⁷³FPLNSYMNA⁸²TNHAIVQTLVHFI⁹⁵NPETVPKP¹⁰³--- | (SEQ ID NO: 44 wild-type) |
| ---W⁷³TQWNSYMNA⁸²TNHAIVQTLVHFI⁹⁵NPETVPKP¹⁰³--- | (SEQ ID NO: 45) |
| ---A⁷³WTQWSYMNA⁸²TNHAIVQTLVHFI⁹⁵NPETVPKP¹⁰³--- | (SEQ ID NO: 46) |
| ---A⁷³FWTQWYMNA⁸²TNHAIVQTLVHFI⁹⁵NPETVPKP¹⁰³--- | (SEQ ID NO: 47) |
| ---A⁷³FPWTQWMNA⁸²TNHAIVQTLVHFI⁹⁵NPETVPKP¹⁰³--- | (SEQ ID NO: 48) |
| ---A⁷³FPLNWTQWA⁸²TNHAIVQTLVHFI⁹⁵NPETVPKP¹⁰³--- | (SEQ ID NO: 49) |
| ---A⁷³FPLNWTQWA⁸²TNHAIVQTLVHFI⁹⁵NPETVPKP¹⁰³--- | (SEQ ID NO: 50) |
| ---A⁷³FPLNSWTQW⁸²TNHAIVQTLVHFI⁹⁵NPETVPKP¹⁰³--- | (SEQ ID NO: 51) |
| Substitution of existing amino acids I⁹⁵ to P¹⁰³ | |
| ---A⁷³FPLNSYMNA⁸²TNHAIVQTLVHFW⁹⁵TQWTVPKP¹⁰³--- | (SEQ ID NO: 52) |
| ---A⁷³FPLNSYMNA⁸²TNHAIVQTLVHFI⁹⁵WTQWVPKP¹⁰³--- | (SEQ ID NO: 53) |
| ---A⁷³FPLNSYMNA⁸²TNHAIVQTLVHFI⁹⁵NWTQWPKP¹⁰³--- | (SEQ ID NO: 54) |
| ---A⁷³FPLNSYMNA⁸²TNHAIVQTLVHFI⁹⁵NPWTQWKP¹⁰³--- | (SEQ ID NO: 55) |
| ---A⁷³FPLNSYMNA⁸²TNHAIVQTLVHFI⁹⁵NPEWTQWP¹⁰³--- | (SEQ ID NO: 56) |
| ---A⁷³FPLNSYMNA⁸²TNHAIVQTLVHFI⁹⁵NPETWTQW¹⁰³--- | (SEQ ID NO: 57) |
| Insertion (with two amino acids added) between existing amino acids A⁷³ to A⁸² | |
| ---W⁷³TQWPLNSYMNA⁸²TNHAIVQTLVHFI⁹⁵NPETVPKP¹⁰³--- | (SEQ ID NO: 58) |
| ---A⁷³WTQWLNSYMNA⁸²TNHAIVQTLVHFI⁹⁵NPETVPKP¹⁰³--- | (SEQ ID NO: 59) |
| ---A⁷³FWTQWNSYMNA⁸²TNHAIVQTLVHFI⁹⁵NPETVPKP¹⁰³--- | (SEQ ID NO: 60) |
| ---A⁷³FPWTQWSYMNA⁸²TNHAIVQTLVHFI⁹⁵NPETVPKP¹⁰³--- | (SEQ ID NO: 61) |
| ---A⁷³FPLWTQWYMNA⁸²TNHAIVQTLVHFI⁹⁵NPETVPKP¹⁰³--- | (SEQ ID NO: 62) |
| ---A⁷³FPLNWTQWMNA⁸²TNHAIVQTLVHFI⁹⁵NPETVPKP¹⁰³--- | (SEQ ID NO: 63) |
| ---A⁷³FPLNSWTQWNA⁸²TNHAIVQTLVHFI⁹⁵NPETVPKP¹⁰³--- | (SEQ ID NO: 64) |
| ---A⁷³FPLNSYWTQWA⁸²TNHAIVQTLVHFI⁹⁵NPETVPKP¹⁰³--- | (SEQ ID NO: 65) |
| ---A⁷³FPLNSYMWTQW⁸²TNHAIVQTLVHFI⁹⁵NPETVPKP¹⁰³--- | (SEQ ID NO: 66) |
| Insertion (with one amino acid added) between existing amino acids I⁹⁵ to P¹⁰³ | |
| ---A⁷³TPLNSYMNA⁸²TNHAIVQTLVHFW⁹⁵TQWPETVPKP¹⁰³--- | (SEQ ID NO: 67) |
| ---A⁷³FPLNSYMNA⁸²TNHAIVQTLVHFI⁹⁵WTQWETVPKP¹⁰³--- | (SEQ ID NO: 68) |
| ---A⁷³FPLNSYMNA⁸²TNHAIVQTLVHFI⁹⁵NWTQWTVPKP¹⁰³--- | (SEQ ID NO: 69) |
| ---A⁷³FPLNSYMNA⁸²TNHAIVQTLVHFI⁹⁵NPWTQWVPKP¹⁰³--- | (SEQ ID NO: 70) |
| ---A⁷³FPLNSYMNA⁸²TNHAIVQTLVHFI⁹⁵NPEWTQWPKP¹⁰³--- | (SEQ ID NO: 71) |
| ---A⁷³FPLNSYMNA⁸²TNHAIVQTLVHFI⁹⁵NPETWTQWKP¹⁰³--- | (SEQ ID NO: 72) |
| ---A⁷³FPLNSYMNA⁸²TNHAIVQTLVHFI⁹⁵NPETVWTQWP¹⁰³--- | (SEQ ID NO: 73) |
| ---A⁷³FPLNSYMNA⁸²TNHAIVQTLVHFI⁹⁵NPETVPWTQW¹⁰³--- | (SEQ ID NO: 74) |

The same substitutions and insertions can be generated using any other consensus sequences of the invention, such as SEQ ID NOs: 1 through 12. Likewise, mutants of another wild type peptide can be generated using an analogous approach together with any of the consensus sequences of the invention.

Appropriate C- and N-glycosylation consensus sequences for BMP-7 and peptides other than BMP-7 can be determined by (a) preparing a polypeptide that incorporates a putative glycosylation consensus sequence and (b) submitting that polypeptide to suitable glycosylation conditions (e.g, to an in vitro assay system), thereby confirming its ability to serve as a substrate for the respective glycosyltransferase (e.g., C-mannosyltransferase).

Once a variety of mutants are prepared, they can be evaluated for their ability to function as a substrate for C- and/or N-glycosylation (e.g., C- and N-mannosylation), in a screening effort, using a reaction procedure that involves, for instance, a C- or N-mannosyltransferase. In one example, successful glycosylation is detected using methods known in the art, such as mass spectroscopy (e.g., MALDI-TOF or Q-TOF). After identification of those mutants that are suitable substrates for respective glycosyltransferases, production of those mutants and glycosylation reactions can be scaled up. In one embodiment, additional sugar residues including mono- and oligosaccharides are added to a glycosylated peptide using a glycosyltransferase that is known to add to the existing glycosyl residue (e.g., mannose). Together these methods may result in glycosyl structures including two or more sugar residues that are bound to the engineered glycosylation site.

Moreover, as will be apparent to one of skill in the art, peptides that include one or more mutation are within the scope of the present invention. Additional mutations may be introduced to increase the number of glycosylation sites, and to adjust solubility, molecular size, biological activity, immunogenicity, metabolic stability etc. of the polypeptide.

### Glycosyl Linking Group

The saccharide component of the modified sugar, when interposed between the peptide and a selected moiety, becomes a “glycosyl linking group.” The glycosyl linking group is formed from any mono- or oligo-saccharide that, after modification with a selected moiety, is a substrate for an appropriate glycosyltransferase. The polypeptide conjugates of the invention can include glycosyl linking groups that are mono- or multi-valent (e.g., antennary structures). Thus, conjugates of the invention include species in which a selected moiety is attached to a peptide via a monovalent glycosyl linking group. Also included within the invention are conjugates in which more than one selected modifying groups are attached to a peptide via a multivalent linking group.

In one embodiment, X* in Formula (I) is a member selected from a sialyl moiety (Sia), a galactosyl moiety (Gal) and combinations thereof. In an exemplary embodiment, X* is a -Gal-Sia moiety. In another embodiment, X* in Formula (I) includes a moiety according to Formula (II):

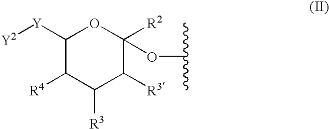

In Formula (II), R² is a member selected from H, —R¹, —CH₂R¹, and —C(X¹)R¹ wherein R¹ is a member selected from OR⁹, SR⁹, NR¹⁰R¹¹, substituted or unsubstituted alkyl and substituted or unsubstituted heteroalkyl. R⁹ is a member independently selected from H, a metal ion, substituted or unsubstituted alkyl, substituted or unsubstituted heteroalkyl and acyl. R¹⁰ is a member selected from H, substituted or unsubstituted alkyl, substituted or unsubstituted heteroalkyl and acyl. X¹ is a member selected from substituted or unsubstituted alkyl, O, S and NR⁸ wherein R⁸ is a member selected from H, OH, substituted or unsubstituted alkyl and substituted or unsubstituted heteroalkyl.

Y is a member selected from CH₂, CH(OH)CH₂, CH(OH)CH(OH)CH₂, CH, CH(OH)CH; CH(OH)CH(OH)CH, CH(OH), CH(OH)CH(OH), and CH(OH)CH(OH)CH(OH), and Y² is a member selected from H, OH, R⁶, substituted or unsubstituted alkyl, substituted or unsubstituted heteroalkyl,

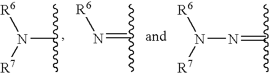

wherein R⁶ and R⁷ are members independently selected from H, L-R^{6b}, C(O)R^{6b}, C(O)-L^{a}—R^{6b}, substituted or unsubstituted alkyl and substituted or unsubstituted heteroalkyl wherein L^{a} is a member selected from a bond and a linker group, and R^{6b} is a member selected from H, substituted or unsubstituted alkyl, substituted or unsubstituted heteroalkyl and a modifying group.

R³ is a member selected from H, substituted or unsubstituted alkyl, substituted or unsubstituted heteroalkyl, and OR^{3″}, wherein R^{3″} is a member selected from H, substituted or unsubstituted alkyl and substituted or unsubstituted heteroalkyl. R^{3′} and R⁴ are members independently selected from H, OH, substituted or unsubstituted alkyl, substituted or unsubstituted heteroalkyl, L^{a}-R^{6c}, C(O)R^{6c}, C(O) L^{a}-R^{6c}, NHC(O)R^{6c}, member selected from a bond and a linker group. R^{6c} is a member selected from H, substituted or unsubstituted alkyl, substituted or unsubstituted heteroalkyl, substituted or unsubstituted aryl, substituted or unsubstituted heteroaryl, substituted or unsubstituted heterocycloalkyl, NR¹³R¹⁴ and a modifying group, wherein R¹³ and R¹⁴ are members independently selected from H, substituted or unsubstituted alkyl and substituted or unsubstituted heteroalkyl.

In another exemplary embodiment, X* in Formula (I) includes a moiety according to Formula (III):

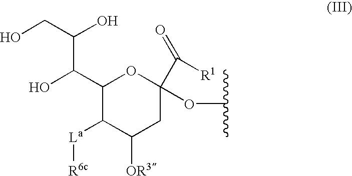

wherein L^{a}, R¹ and R^{6c} are defined as in Formula (II) above.

Exemplary R^{6c} groups include the following structures:

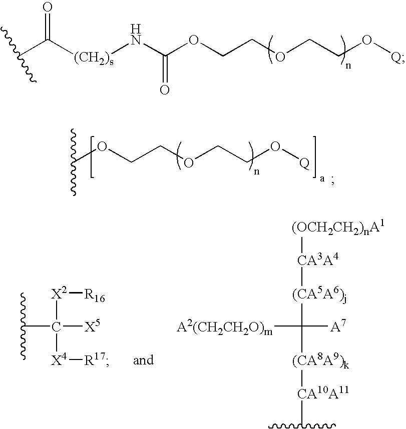

wherein the indices m and n are integers independently selected from 0 to 5000, s is an integer from 0 to 20, a is an integer from 1-5 and the indices j and k are independently selected from 0 to 20. Q is a member selected from H and C₁-C₆ alkyl, R¹⁶ and R¹⁷ are independently selected polymeric moieties, X² and X⁴ are independently selected linkage fragments joining polymeric moieties R¹⁶ and R¹⁷ to C, and X⁵ is a non-reactive group.

A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Additional modifying groups of the invention are discussed herein below.

### Sulfur-Linked Peptide Conjugates

In a further aspect, the invention provides a peptide conjugate that includes: (a) a peptide, and (b) a modifying group, wherein the modifying group is covalently attached to the peptide at a glycosyl or amino acid residue of the polypeptide via a glycosyl linking group, wherein the glycosyl linking group includes at least one thioglycosidic bond.

In an exemplary embodiment, the glycosyl linking group with a thioglycosidic bond includes a moiety according to Formula (V):

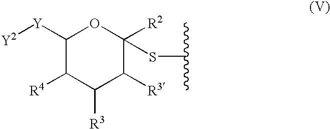

In Formula (V), R², R³, R^{3′}, R⁴, Y and Y² are as defined for Formula (II) above.

In another exemplary embodiment, the glycosyl linking group with a thioglycosidic bond includes a moiety according to Formula (VI):

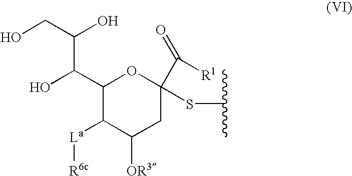

wherein L^{a}, R¹ and R^{6c} are defined as in Formula (III) above.

### Exemplary Polypeptide Conjugates

Exemplary polypeptide conjugates of the invention (including carbon- and nitrogen-linked conjugates as well as sulfur-linked conjugates) are derived from: bone morphogenetic protein (e.g., BMP-2, BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (ATT, or α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), TNF receptor-IgG Fc region fusion protein (Enbrel™), anti-HER2 monoclonal antibody (Herceptin™), monoclonal antibody to protein F of respiratory syncytial virus (Synagis™), monoclonal antibody to TNF-α (Remicade™), monoclonal antibody to glycoprotein IIb/IIIa (Reopro™), monoclonal antibody to CD20 (Rituxan™), anti-thrombin III (AT III), human chorionic gonadotropin (hCG), alpha-galactosidase (Fabrazyme™), alpha-iduronidase (Aldurazyme™), follicle stimulating hormone (FSH), beta-glucosidase, anti-TNF-alpha monoclonal antibody (MLB 5075), glucagon-like peptide-1 (GLP-1), beta-glucosidase (MLB 5064), alpha-galactosidase A (MLB 5082), fibroblast growth factor (FGF), Factor VII, Factor VIII, Factor IX, prokinetisin and extendin-4, as well as any modified versions (e.g., mutants) thereof.

In an exemplary embodiment, the polypeptide is an interferon. The interferons are antiviral glycoproteins that, in humans, are secreted by human primary fibroblasts after induction with virus or double-stranded RNA. Interferons are of interest as therapeutics, e.g, antiviral agents (e.g., hepatitis B and C), antitumor agents (e.g., hepatocellular carcinoma) and in the treatment of multiple sclerosis. For references relevant to interferon-α, see, Asano, et al., Eur. J. Cancer, 27(Suppl 4):S21-S25 (1991); Nagy, et al., Anticancer Research, 8(3):467-470 (1988); Dron, et al., J. Biol. Regul. Homeost. Agents, 3(1):13-19 (1989); Habib, et al., Am. Surg., 67(3):257-260 (3/2001); and Sugyiama, et al., Eur. J. Biochem., 217:921-927 (1993). For references discussing interferon-β, see, e.g., Yu, et al., J. Neuroimmunol., 64(1):91-100 (1996); Schmidt, J., J. Neurosci. Res., 65(1):59-67 (2001); Wender, et al., Folia Neuropathol., 39(2):91-93 (2001); Martin, et al., Springer Semin. Immunopathol., 18(1):1-24 (1996); Takane, et al., J. Pharmacol. Exp. Ther., 294(2):746-752 (2000); Sburlati, et al., Biotechnol. Prog., 14:189-192 (1998); Dodd, et al., Biochimica et Biophysica Acta, 787:183-187 (1984); Edelbaum, et al., J. Interferon Res., 12:449-453 (1992); Conradt, et al., J. Biol. Chem., 262(30):14600-14605 (1987); Civas, et al., Eur. J. Biochem., 173:311-316 (1988); Demolder, et al., J. Biotechnol., 32:179-189 (1994); Sedmak, et al., J. Interferon Res., 9(Suppl 1):S61-S65 (1989); Kagawa, et al., J. Biol. Chem., 263(33):17508-17515 (1988); Hershenson, et al., U.S. Pat. No. 4,894,330; Jayaram, et al., J. Interferon Res., 3(2):177-180 (1983); Menge, et al., Develop. Biol. Standard., 66:391-401 (1987); Vonk, et al., J. Interferon Res., 3(2):169-175 (1983); and Adolf, et al., J. Interferon Res., 10:255-267 (1990).

In an exemplary interferon conjugate, interferon alpha, e.g., interferon alpha 2b and 2a, is conjugated to a water soluble polymer through an intact glycosyl linking group.

In a further exemplary embodiment, the invention provides a conjugate of human granulocyte colony stimulating factor (G-CSF). G-CSF is a glycoprotein that stimulates proliferation, differentiation and activation of neutropoietic progenitor cells into functionally mature neutrophils. Injected G-CSF is rapidly cleared from the body. See, for example, Nohynek, et al., Cancer Chemother. Pharmacol., 39:259-266 (1997); Lord, et al., Clinical Cancer Research, 7(7):2085-2090 (07/2001); Rotondaro, et al., Molecular Biotechnology, 11(2):117-128 (1999); and Bonig, et al., Bone Marrow Transplantation, 28: 259-264 (2001).

The present invention encompasses a method for the modification of GM-CSF. GM-CSF is well known in the art as a cytokine produced by activated T-cells, macrophages, endothelial cells, and stromal fibroblasts. GM-CSF primarily acts on the bone marrow to increase the production of inflammatory leukocytes, and further functions as an endocrine hormone to initiate the replenishment of neutrophils consumed during inflammatory functions. Further GM-CSF is a macrophage-activating factor and promotes the differentiation of Lagerhans cells into dendritic cells. Like G-CSF, GM-CSF also has clinical applications in bone marrow replacement following chemotherapy

### Modified Sugars

Modified glycosyl donor species (“modified sugar donors”) are preferably selected from modified sugar nucleotides, activated modified sugars and modified sugars that are simple saccharides that are neither nucleotides nor activated. Any desired carbohydrate structure can be added to a peptide using the methods of the invention. Typically, the structure will be a monosaccharide, but the present invention is not limited to the use of modified monosaccharide sugars; oligosaccharides and polysaccharides are useful as well.

The modifying group is attached to a sugar moiety by enzymatic means, chemical means or a combination thereof, thereby producing a modified sugar. The sugars are substituted at any position that allows for the attachment of the modifying group, yet which still allows the sugar to function as a substrate for the enzyme used to ligate the modified sugar to the peptide. In an exemplary embodiment, when sialic acid is the sugar, the sialic acid is substituted with the modifying group at either the pyruvyl side chain or at the 5-position on the amine moiety that is normally acetylated in sialic acid.

### Sugar Nucleotides

In certain embodiments of the present invention, a modified sugar nucleotide is utilized to add the modified sugar to the peptide. Exemplary sugar nucleotides that are used in the present invention in their modified form include nucleotide mono-, di- or triphosphates or analogs thereof. In another embodiment, the modified sugar nucleotide is selected from a UDP-glycoside, CMP-glycoside, and a GDP-glycoside. Even more preferably, the modified sugar nucleotide is selected from an UDP-galactose, UDP-galactosamine, UDP-glucose, UDP-glucosamine, GDP-mannose, GDP-fucose, CMP-sialic acid, and CMP-NeuAc. N-acetylamine derivatives of the sugar nucleotides are also of use in the method of the invention.

In one example, the nucleotide sugar species is modified with a water-soluble polymer. An exemplary modified sugar nucleotide bears a sugar group that is modified through an amine moiety on the sugar. Modified sugar nucleotides, e.g., saccharyl-amine derivatives of a sugar nucleotide, are also of use in the methods of the invention. For example, a saccharyl amine (without the modifying group) can be enzymatically conjugated to a peptide (or other species) and the free saccharyl amine moiety subsequently conjugated to a desired modifying group. Alternatively, the modified sugar nucleotide can function as a substrate for an enzyme that transfers the modified sugar to a saccharyl acceptor on the polypeptide.

In an exemplary embodiment, the modified sugar is based upon a 6-amino-N-acetyl-glycosyl moiety. As shown below for N-acetylgalactosamine, the 6-amino-sugar moiety is readily prepared by standard methods.

In the scheme above, the index n represents an integer from 0 to 2500, preferably from 10 to 1500, and more preferably from 10 to 1200. The symbol “A” represents an activating group, e.g., a halo, a component of an activated ester (e.g., a N-hydroxysuccinimide ester), a component of a carbonate (e.g., p-nitrophenyl carbonate) and the like. Those of skill in the art will appreciate that other PEG-amide nucleotide sugars are readily prepared by this and analogous methods.

In other exemplary embodiments, the amide moiety is replaced by a group such as a urethane or a urea.

### Activated Sugars

In other embodiments, the modified sugar is an activated sugar. Activated, modified sugars, which are useful in the present invention are typically glycosides which have been synthetically altered to include a leaving group. In one example, the activated sugar is used in an enzymatic reaction to transfer the activated sugar onto an acceptor on the peptide or glycopeptide. In another example, the activated sugar is added to the peptide or glycopeptide by chemical means. Thus, a “leaving group” refers to those moieties, which are easily displaced in enzyme-regulated nucleophilic substitution reactions or alternatively, are replaced in a chemical reaction utilizing a nucleophilic reaction partner (e.g., a glycosyl moiety carrying a sufhydryl group). It is within the abilities of a skilled person to select a suitable leaving group for each type of reaction. Many activated sugars are known in the art. See, for example, Vocadlo et al., In CARBOHYDRATE CHEMISTRY AND BIOLOGY, Vol. 2, Ernst et al. Ed., Wiley-VCH Verlag: Weinheim, Germany, 2000; Kodama et al., Tetrahedron Lett. 34: 6419 (1993); Lougheed, et al., J. Biol. Chem. 274: 37717 (1999)).

Examples of activating groups (leaving groups) include fluoro, chloro, bromo, tosylate ester, mesylate ester, triflate ester and the like. Preferred leaving groups, for use in enzyme mediated reactions, are those that do not significantly sterically encumber the enzymatic transfer of the glycoside to the acceptor. Accordingly, preferred embodiments of activated glycoside derivatives include glycosyl fluorides and glycosyl mesylates, with glycosyl fluorides being particularly preferred. Among the glycosyl fluorides, α-galactosyl fluoride, α-mannosyl fluoride, α-glucosyl fluoride, α-fucosyl fluoride, α-xylosyl fluoride, α-sialyl fluoride, α-N-acetylglucosaminyl fluoride, α-N-acetylgalactosaminyl fluoride, β-galactosyl fluoride, β-mannosyl fluoride, β-glucosyl fluoride, β-fucosyl fluoride, β-xylosyl fluoride, β-sialyl fluoride, β-N-acetylglucosaminyl fluoride and β-N-acetylgalactosaminyl fluoride are most preferred. For non-enzymatic, nucleophilic substitutions, these and other leaving groups may be useful. For instance, the activated donor glycoside can be a dinitrophenyl (DNP), or bromo-glycoside.

By way of illustration, glycosyl fluorides can be prepared from the free sugar by first acetylating and then treating the sugar moiety with HF/pyridine. This generates the thermodynamically most stable anomer of the protected (acetylated) glycosyl fluoride (i.e., the α-glycosyl fluoride). If the less stable anomer (i.e., the β-glycosyl fluoride) is desired, it can be prepared by converting the peracetylated sugar with HBr/HOAc or with HCl to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g. NaOMe/MeOH). In addition, many glycosyl fluorides are commercially available.

Other activated glycosyl derivatives can be prepared using conventional methods known to those of skill in the art. For example, glycosyl mesylates can be prepared by treatment of the fully benzylated hemiacetal form of the sugar with mesyl chloride, followed by catalytic hydrogenation to remove the benzyl groups.

In a further exemplary embodiment, the modified sugar is an oligosaccharide having an antennary structure. In another embodiment, one or more of the termini of the antennae bear the modifying moiety. When more than one modifying moiety is attached to an oligosaccharide having an antennary structure, the oligosaccharide is useful to “amplify” the modifying moiety; each oligosaccharide unit conjugated to the peptide attaches multiple copies of the modifying group to the peptide. The general structure of a typical conjugate of the invention as set forth in the drawing above encompasses multivalent species resulting from preparing a conjugate of the invention utilizing an antennary structure. Many antennary saccharide structures are known in the art, and the present method can be practiced with them without limitation.

### Modifying Groups

The modifying group of the invention can be any chemical moiety. Exemplary modifying groups are discussed below. The modifying groups can be selected for their ability to alter the properties (e.g., biological or physicochemical properties) of a given polypeptide. Exemplary properties that may be altered by the use of modifying groups include, but are not limited to, pharmacokinetics, pharmacodynamics, metabolic stability, biodistribution, water solubility, lipophilicity, and tissue targeting capabilities. Preferred modifying groups are those which improve pharmacodynamics and pharmacokinetics of a pharmaceutical polypeptide that has been modified with such modifying group. Other modifying groups may be useful for the modification of peptides that are used in diagnostic applications or in vitro biological assay systems.

For example, the in vivo half-life of therapeutic glycopeptides can be enhanced with polyethylene glycol (PEG) moieties. Chemical modification of polypeptides with PEG (PEGylation) increases their molecular size and typically decreases surface- and functional group-accessibility, each of which are dependent on the number and size of the PEG moieties attached to the polypeptide. Frequently, this modification results in an improvement of plasma half-live and in proteolytic-stability, and a decrease in immunogenicity and hepatic uptake (Chaffee et al. J. Clin. Invest. 89: 1643-1651 (1992); Pyatak et al. Res. Commun. Chem. Pathol Pharmacol. 29: 113-127 (1980)). PEGylation of interleukin-2 has been reported to increase its antitumor potency in vivo (Katre et al. Proc. Natl. Acad. Sci. USA. 84: 1487-1491 (1987)) and PEGylation of a F(ab′)2 derived from the monoclonal antibody A7 has improved its tumor localization (Kitamura et al. Biochem. Biophys. Res. Commun. 28: 1387-1394 (1990)). Thus, in another embodiment, the in vivo half-life of a peptide derivatized with a PEG moiety by a method of the invention is increased relevant to the in vivo half-life of the non-derivatized peptide.

The increase in peptide in vivo half-life is best expressed as a range of percent increase in this quantity. The lower end of the range of percent increase is about 40%, about 60%, about 80%, about 100%, about 150% or about 200%. The upper end of the range is about 60%, about 80%, about 100%, about 150%, or more than about 250%.

### Water-Soluble Polymeric Modifying Groups

Many water-soluble polymers are known to those of skill in the art and are useful in practicing the present invention. The term water-soluble polymer encompasses species such as saccharides (e.g., dextran, amylose, hyalouronic acid, poly(sialic acid), heparans, heparins, etc.); poly(amino acids), e.g., poly(aspartic acid) and poly(glutamic acid); nucleic acids; synthetic polymers (e.g., poly(acrylic acid), poly(ethers), e.g., poly(ethylene glycol); peptides, proteins, and the like. The present invention may be practiced with any water-soluble polymer with the sole limitation that the polymer must include a point at which the remainder of the conjugate can be attached.

The use of reactive derivatives of PEG (or other linkers) to attach one or more peptide moieties to the linker is within the scope of the present invention. The invention is not limited by the identity of the reactive PEG analogue. Many activated derivatives of poly(ethyleneglycol) are available commercially and in the literature. It is well within the abilities of one of skill to choose, and synthesize if necessary, an appropriate activated PEG derivative with which to prepare a substrate useful in the present invention. See, Abuchowski et al. Cancer Biochem. Biophys., 7: 175-186 (1984); Abuchowski et al., J. Biol. Chem., 252: 3582-3586 (1977); Jackson et al., Anal. Biochem., 165: 114-127 (1987); Koide et al., Biochem Biophys. Res. Commun., 111: 659-667 (1983)), tresylate (Nilsson et al., Methods Enzymol., 104: 56-69 (1984); Delgado et al., Biotechnol. Appl. Biochem., 12: 119-128 (1990)); N-hydroxysuccinimide derived active esters (Buckmann et al., Makromol. Chem., 182: 1379-1384 (1981); Joppich et al., Makromol. Chem., 180: 1381-1384 (1979); Abuchowski et al., Cancer Biochem. Biophys., 7: 175-186 (1984); Katre et al. Proc. Natl. Acad. Sci. U.S.A., 84: 1487-1491 (1987); Kitamura et al., Cancer Res., 51: 4310-4315 (1991); Boccu et al., Z. Naturforsch., 38C: 94-99 (1983), carbonates (Zalipsky et al., POLY(ETHYLENE GLYCOL) CHEMISTRY: BIOTECHNICAL AND BIOMEDICAL APPLICATIONS, Harris, Ed., Plenum Press, New York, 1992, pp. 347-370; Zalipsky et al., Biotechnol. Appl. Biochem., 15: 100-114 (1992); Veronese et al., Appl Biochem. Biotech., 11: 141-152 (1985)), imidazolyl formates (Beauchamp et al., Anal. Biochem., 131: 25-33 (1983); Berger et al., Blood, 71: 1641-1647 (1988)), 4-dithiopyridines (Woghiren et al., Bioconjugate Chem., 4: 314-318 (1993)), isocyanates (Byun et al., ASAIO Journal, M649-M-653 (1992)) and epoxides (U.S. Pat. No. 4,806,595, issued to Noishiki et al., (1989). Other linking groups include the urethane linkage between amino groups and activated PEG. See, Veronese, et al., Appl Biochem. Biotechnol., 11: 141-152 (1985).

Methods for activation of polymers can be found in WO 94/17039, U.S. Pat. No. 5,324,844, WO 94/18247, WO 94/04193, U.S. Pat. No. 5,219,564, U.S. Pat. No. 5,122,614, WO 90/13540, U.S. Pat. No. 5,281,698, and more WO 93/15189, and for conjugation between activated polymers and peptides, e.g. Coagulation Factor VIII (WO 94/15625), hemoglobin (WO 94/09027), oxygen carrying molecule (U.S. Pat. No. 4,412,989), ribonuclease and superoxide dismutase (Veronese at al., App. Biochem. Biotech. 11: 141-45 (1985)).

In an exemplary embodiment in which a reactive PEG derivative is utilized, the invention provides a method for extending the blood-circulation half-life of a selected peptide, in essence targeting the peptide to the blood pool, by conjugating the peptide to a synthetic or natural polymer of a size sufficient to retard the filtration of the protein by the glomerulus (e.g., albumin). This embodiment of the invention is illustrated in Scheme 3, in which G-CSF is conjugated to albumin via a PEG linker using a combination of chemical and enzymatic modifications.

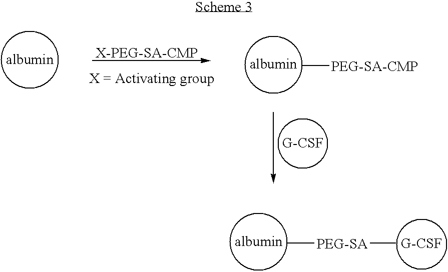

As shown in Scheme 3, a residue (e.g., amino acid side chain) of albumin is modified with a reactive PEG derivative, such as X-PEG-(CMP-sialic acid), in which X is an activating group (e.g, active ester, isothiocyanate, etc). The PEG derivative and G-CSF are combined and contacted with a transferase for which CMP-sialic acid is a substrate. In a further illustrative embodiment, an ε-amine of lysine is reacted with the N-hydroxysuccinimide ester of the PEG-linker to form the albumin conjugate. The CMP-sialic acid of the linker is enzymatically conjugated to an appropriate residue on GCSF, e.g, Gal, or GalNAc thereby forming the conjugate. Those of skill will appreciate that the above-described method is not limited to the reaction partners set forth. Moreover, the method can be practiced to form conjugates that include more than two protein moieties by, for example, utilizing a branched linker having more than two termini.

Exemplary water-soluble polymers are those in which a substantial proportion of the polymer molecules in a sample of the polymer are of approximately the same molecular weight; such polymers are “homodisperse.”

The present invention is further illustrated by reference to a poly(ethylene glycol) conjugate. Several reviews and monographs on the functionalization and conjugation of PEG are available. See, for example, Harris, Macronol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); Zalipsky, Bioconjugate Chem. 6: 150-165 (1995); and Bhadra, et al., Pharmazie, 57:5-29 (2002). Routes for preparing reactive PEG molecules and forming conjugates using the reactive molecules are known in the art. For example, U.S. Pat. No. 5,672,662 discloses a water soluble and isolatable conjugate of an active ester of a polymer acid selected from linear or branched poly(alkylene oxides), poly(oxyethylated polyols), poly(olefinic alcohols), and poly(acrylomorpholine).

U.S. Pat. No. 6,376,604 sets forth a method for preparing a water-soluble 1-benzotriazolylcarbonate ester of a water-soluble and non-peptidic polymer by reacting a terminal hydroxyl of the polymer with di(1-benzotriazoyl)carbonate in an organic solvent. The active ester is used to form conjugates with a biologically active agent such as a protein or peptide.

WO 99/45964 describes a conjugate comprising a biologically active agent and an activated water soluble polymer comprising a polymer backbone having at least one terminus linked to the polymer backbone through a stable linkage, wherein at least one terminus comprises a branching moiety having proximal reactive groups linked to the branching moiety, in which the biologically active agent is linked to at least one of the proximal reactive groups. Other branched poly(ethylene glycols) are described in WO 96/21469, U.S. Pat. No. 5,932,462 describes a conjugate formed with a branched PEG molecule that includes a branched terminus that includes reactive functional groups. The free reactive groups are available to react with a biologically active species, such as a protein or peptide, forming conjugates between the poly(ethylene glycol) and the biologically active species. U.S. Pat. No. 5,446,090 describes a bifunctional PEG linker and its use in forming conjugates having a peptide at each of the PEG linker termini.

Conjugates that include degradable PEG linkages are described in WO 99/34833; and WO 99/14259, as well as in U.S. Pat. No. 6,348,558. Such degradable linkages are applicable in the present invention.

The art-recognized methods of polymer activation set forth above are of use in the context of the present invention in the formation of the branched polymers set forth herein and also for the conjugation of these branched polymers to other species, e.g., sugars, sugar nucleotides and the like.

An exemplary water-soluble polymer is poly(ethylene glycol), e.g., methoxy-poly(ethylene glycol). The poly(ethylene glycol) used in the present invention is not restricted to any particular form or molecular weight range. For unbranched poly(ethylene glycol) molecules the molecular weight is preferably between 500 and 100,000. A molecular weight of 2000-60,000 is preferably used and more preferably of from about 5,000 to about 40,000.

Examplary poly(ethylene glycol) molecules of the invention include, but are not limited to, those species set forth below.

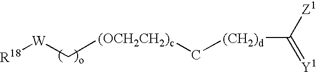

in which R¹⁸ is H, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—CH₂CH₂—, HS—CH₂CH₂—, and —(CH₂)_{q}C(Y¹)Z²; -sugar-nucleotide, or protein. The index “c” represents an integer from 1 to 2500. The indeces d, o, and q independently represent integers from 0 to 20. The symbol Z¹ represents OH, NH₂, halogen, S—R¹⁹, the alcohol portion of activated esters, —(CH₂)_{d1}C(Y³)V, —(CH₂)_{d1}U(CH₂)_{g}C(Y³)ᵥ, sugar-nucleotide, protein, and leaving groups, e.g., imidazole, p-nitrophenyl, HOBT, tetrazole, halide. The symbols X, Y¹, Y³, W, U independently represent the moieties O, S, N—R²⁰. The symbol V represents OH, NH₂, halogen, S—R²¹, the alcohol component of activated esters, the amine component of activated amides, sugar-nucleotides, and proteins. The indeces dl, g and v are members independently selected from the integers from 0 to 20. The symbols R¹⁹, R²⁰ and R²¹ independently represent H, substituted or unsubstituted alkyl, substituted or unsubstituted heteroalkyl, substituted or unsubstituted aryl, substituted or unsubstituted heterocycloalkyl and substituted or unsubstituted heteroaryl.

In another exemplary embodiments, the poly(ethylene glycol) molecule is selected from the following structures:

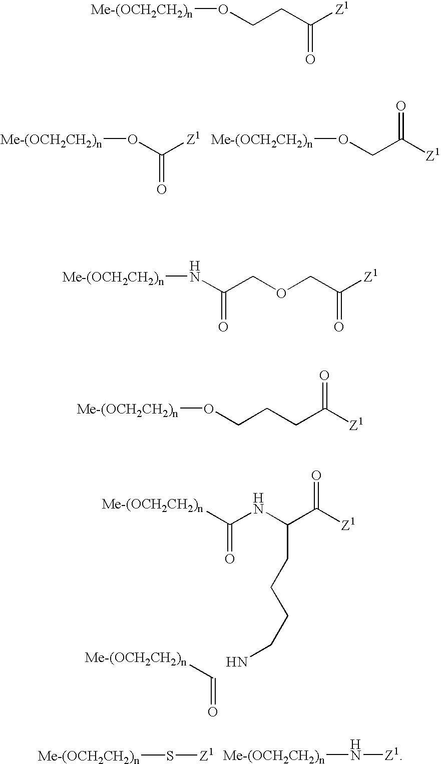

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660; WO 02/09766; Kodera Y., Bioconjugate Chemistry 5: 283-288 (1994); and Yamasaki et al., Agric. Biol. Chem., 52: 2125-2127, 1998. In a preferred embodiment the molecular weight of each poly(ethylene glycol) of the branched PEG is less than or equal to 40,000 daltons.

Representative polymeric modifying moieties include structures that are based on side chain-containing amino acids, e.g., serine, cysteine, lysine, and small peptides, e.g., lys-lys. Exemplary structures include:

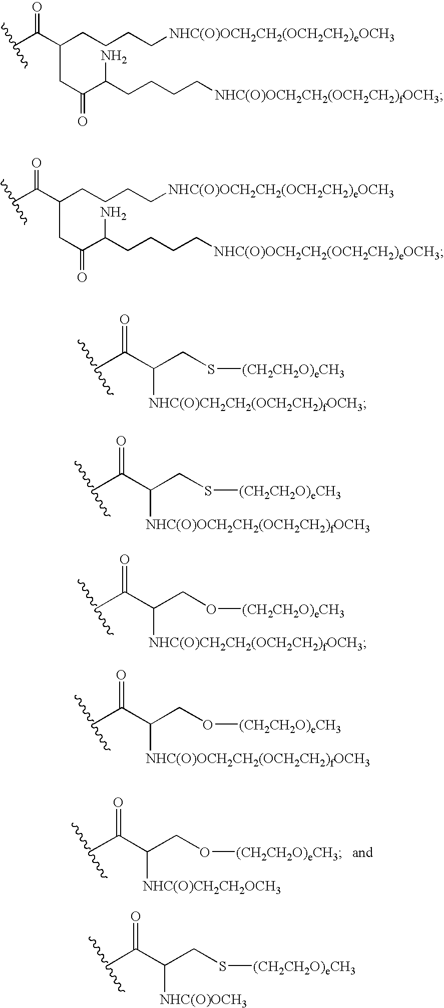

Those of skill will appreciate that the free amine in the di-lysine structures can also be pegylated through an amide or urethane bond with a PEG moiety. In yet another embodiment, the polymeric modifying moiety is a branched PEG moiety that is based upon a tri-lysine peptide. The tri-lysine can be mono-, di-, tri-, or tetra-PEG-ylated. Exemplary species according to this embodiment have the formulae:

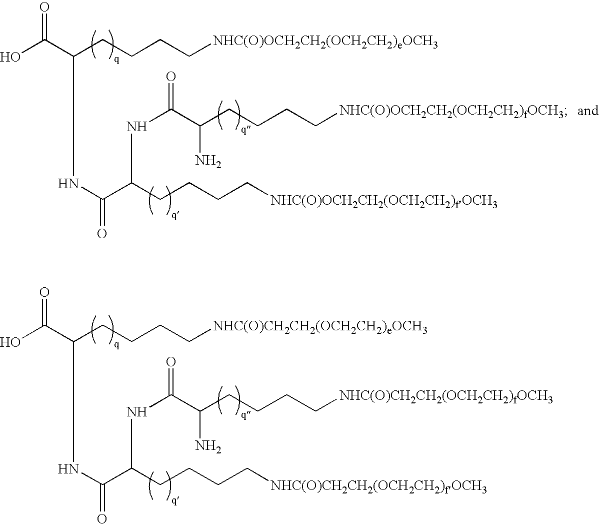

in which the indices e, f and f′ are independently selected integers from 1 to 2500; and the indices q, q′ and q″ are independently selected integers from 1 to 20.

As will be apparent to those of skill, the branched polymers of use in the invention include variations on the themes set forth above. For example the di-lysine-PEG conjugate shown above can include three polymeric subunits, the third bonded to the α-amine shown as unmodified in the structure above. Similarly, the use of a tri-lysine functionalized with three or four polymeric subunits labeled with the polymeric modifying moiety in a desired manner is within the scope of the invention.

As discussed herein, PEG moieties of use in the conjugates of the invention can be linear or branched. An exemplary precursor useful to form a peptide conjugate with a branched modifying group that includes one or more polymeric moiety (e.g., PEG) according to this embodiment has the formula:

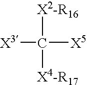

In one embodiment, the branched polymer species according to this formula are essentially pure water-soluble polymers. X^{3′} is a moiety that includes an ionizable (e.g., OH, COOH, H₂PO₄, HSO₃, NH₂, and salts thereof, etc.) or other reactive functional group, e.g., infra. C is carbon. X⁵, R¹⁶ and R¹⁷ are independently selected from non-reactive groups (e.g., H, unsubstituted alkyl, unsubstituted heteroalkyl) and polymeric arms (e.g., PEG). X² and X⁴ are linkage fragments that are preferably essentially non-reactive under physiological conditions, which may be the same or different. An exemplary linker includes neither aromatic nor ester moieties. Alternatively, these linkages can include one or more moiety that is designed to degrade under physiologically relevant conditions, e.g., esters, disulfides, etc. X² and X⁴ join polymeric arms R¹⁶ and R¹⁷ to C. In one embodiment, when X^{3′} is reacted with a reactive functional group of complementary reactivity on a linker, sugar or linker-sugar cassette, X^{3′} is converted to a component of linkage fragment.

Exemplary linkage fragments including X² and X⁴ are independently selected and include S, SC(O)NH, HNC(O)S, SC(O)O, O, NH, NHC(O), (O)CNH and NHC(O)O, and OC(O)NH, CH₂S, CH₂O, CH₂CH₂O, CH₂CH₂S, (CH₂)ₒO, (CH₂)ₒS or (CH₂)ₒY′-PEG wherein, Y′ is S, NH, NHC(O), C(O)NH, NHC(O)O, OC(O)NH, or O and o is an integer from 1 to 50. In an exemplary embodiment, the linkage fragments X² and X⁴ are different linkage fragments.

In an exemplary embodiment, one of the above the precursor or an activated derivative thereof, is reacted with, and thereby bound to a sugar, an activated sugar or a sugar nucleotide through a reaction between X^{3′} and a group of complementary reactivity on the sugar moiety, e.g., an amine. Alternatively, X^{3′} reacts with a reactive functional group on a precursor to linker L^{a} according to Scheme 3.

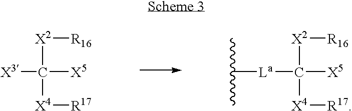

In an exemplary embodiment, the modifying group is derived from a natural or unnatural amino acid, amino acid analogue or amino acid mimetic, or a small peptide formed from one or more such species. For example, certain branched polymers found in the compounds of the invention have the formula:

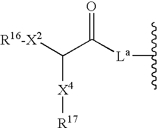

In this example, the linkage fragment C(O)L^{a} is formed by the reaction of a reactive functional group, e.g., X^{3′}, on a precursor of the branched polymeric modifying moiety and a reactive functional group on the sugar moiety, or a precursor to a linker. For example, when X^{3′} is a carboxylic acid, it can be activated and bound directly to an amine group pendent from an amino-saccharide (e.g., Sia, GalNH₂, GlcNH₂, ManNH₂, etc.), forming an amide. Additional exemplary reactive functional groups and activated precursors are described hereinbelow. The symbols have the same identity as those discussed above.

In another exemplary embodiment, L^{a} is a linking moiety having the structure:

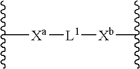

in which X^{a} and X^{b} are independently selected linkage fragments and L¹ is selected from a bond, substituted or unsubstituted alkyl or substituted or unsubstituted heteroalkyl.

Exemplary species for X^{a} and X^{b} include S, SC(O)NH, HNC(O)S, SC(O)O, O, NH, NHC(O), C(O)NH and NHC(O)O, and OC(O)NH.

In another exemplary embodiment, X⁴ is a peptide bond to R¹⁷, which is an amino acid, di-peptide (e.g., Lys-Lys) or tri-peptide (e.g., Lys-Lys-Lys) in which the alpha-amine moiety(ies) and/or side chain heteroatom(s) are modified with a polymeric modifying moiety.

The embodiments of the invention set forth above are further exemplified by reference to species in which the polymer is a water-soluble polymer, particularly poly(ethylene glycol) (“PEG”), e.g., methoxy-poly(ethylene glycol). Those of skill will appreciate that the focus in the sections that follow is for clarity of illustration and the various motifs set forth using PEG as an exemplary polymer are equally applicable to species in which a polymer other than PEG is utilized.

PEG of any molecular weight, e.g. 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa is of use in the present invention.

In other exemplary embodiments, the peptide conjugate includes a moiety selected from the group:

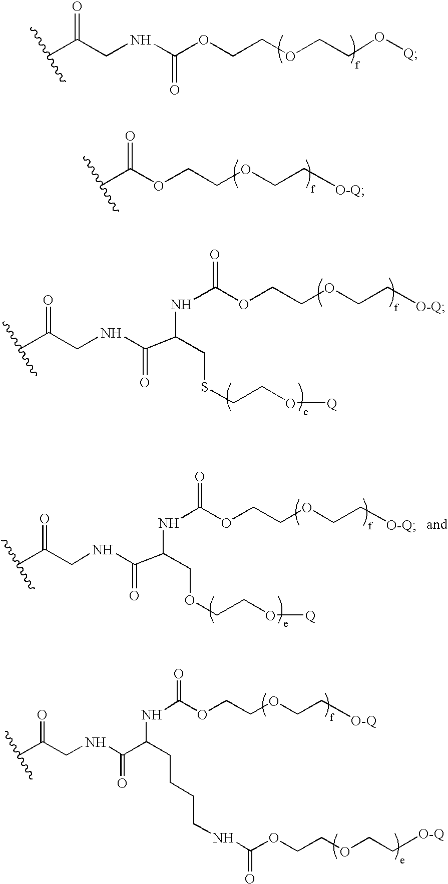

In each of the formulae above, the indices e and f are independently selected from the integers from 1 to 2500. In further exemplary embodiments, e and f are selected to provide a PEG moiety that is about 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa. The symbol Q represents substituted or unsubstituted alkyl (e.g., C₁-C₆ alkyl e.g., methyl), substituted or unsubstituted heteroalkyl or H.

Other branched polymers have structures based on di-lysine (Lys-Lys) peptides, e.g.:

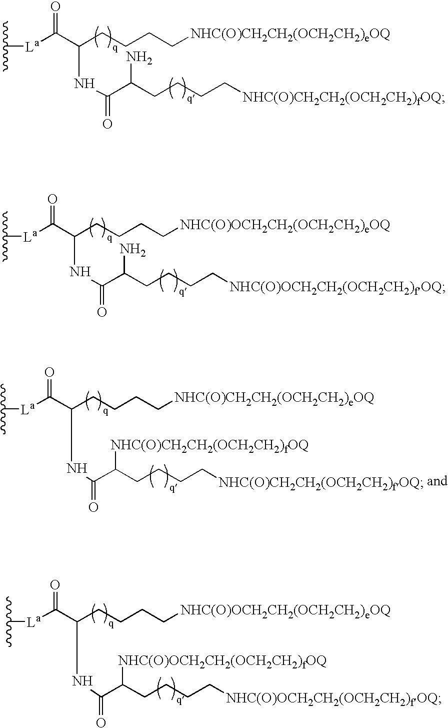

and tri-lysine peptides (Lys-Lys-Lys), e.g.:

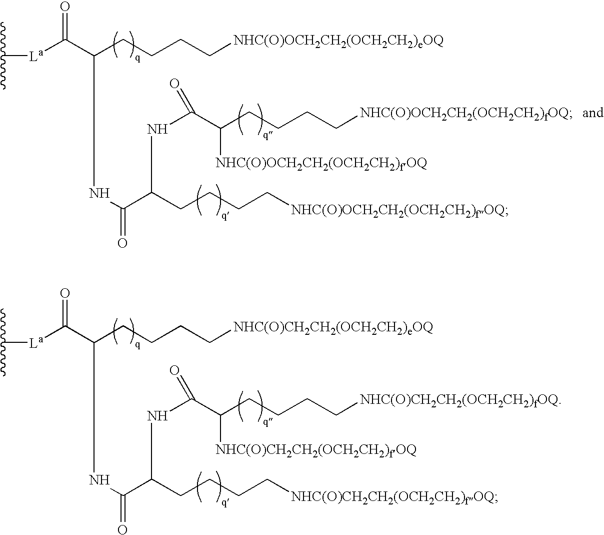

In each of the figures above, the indices e, f, f′ and f″ represent integers independently selected from 1 to 2500. The indices q, q′ and q″ represent integers independently selected from 1 to 20.

In another exemplary embodiment, the conjugates of the invention include a formula which is a member selected from:

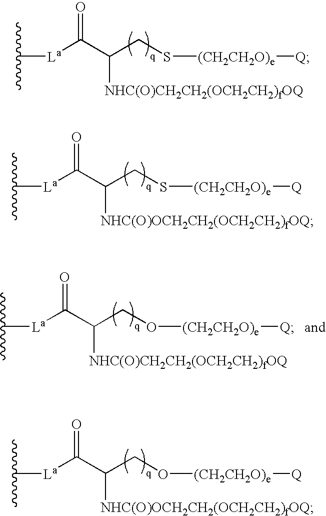

wherein Q is a member selected from H and substituted or unsubstituted C₁-C₆ alkyl. The indices e and f are integers independently selected from 1 to 2500, and the index q is an integer selected from 0 to 20.

In another exemplary embodiment, the conjugates of the invention include a formula which is a member selected from:

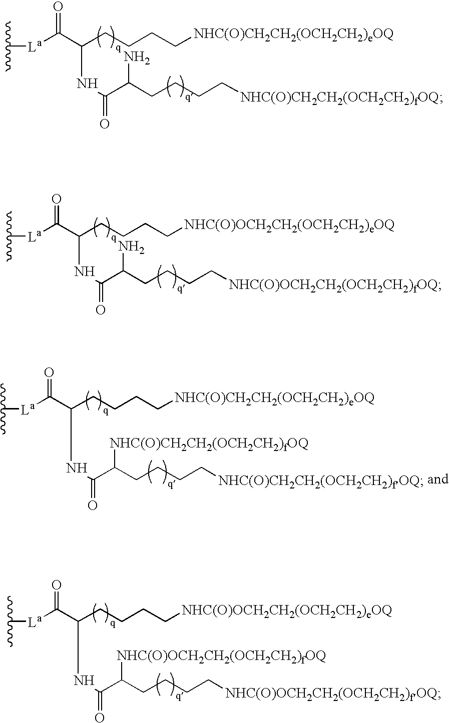

wherein Q is a member selected from H and substituted or unsubstituted C₁-C₆ alkyl, preferably Me. The indices e, f and f″ are integers independently selected from 1 to 2500, and q and q′ are integers independently selected from 1 to 20.

In another exemplary embodiment, the conjugate of the invention includes a structure according to the following formula:

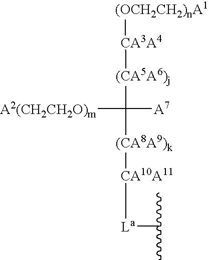

wherein the indices m and n are integers independently selected from 0 to 5000. The indices t and a are independently selected from 0 or 1. The indices j and k are integers independently selected from 0 to 20. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In one embodiment according to the formula above, the branched polymer has a structure according to the following formula:

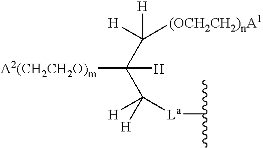

In an exemplary embodiment, A¹ and A² are members independently selected from —OCH₃ and OH.

In another exemplary embodiment, the linker L^{a} is a member selected from aminoglycine derivatives. Exemplary polymeric modifying group according to this embodiment have a structure according to the following formulae:

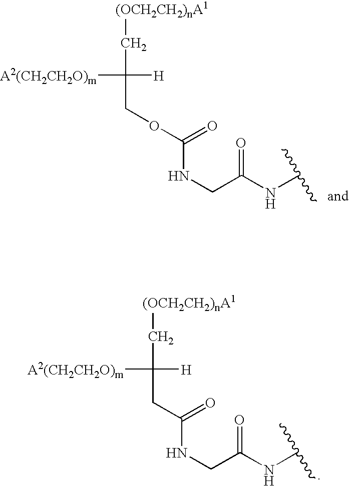

In one example, A¹ and A² are members independently selected from OCH₃ and OH. Exemplary polymeric modifying groups according to this example include:

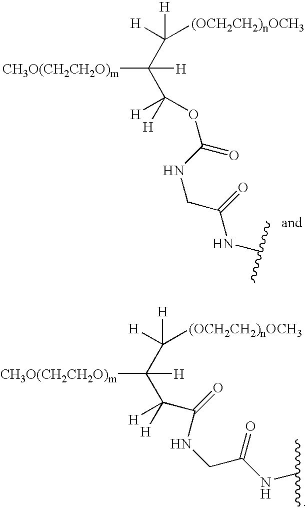

Activated PEG molecules useful in the present invention and methods of making those reagents are known in the art and are described, for example, in WO04/083259.

### Water-Insoluble Polymers

In another embodiment, analogous to those discussed above, the modified sugars include a water-insoluble polymer, rather than a water-soluble polymer. The conjugates of the invention may also include one or more water-insoluble polymers. This embodiment of the invention is illustrated by the use of the conjugate as a vehicle with which to deliver a therapeutic peptide in a controlled manner. Polymeric drug delivery systems are known in the art. See, for example, Dunn et al., Eds. POLYMERIC DRUGS AND DRUG DELIVERY SYSTEMS, ACS Symposium Series Vol. 469, American Chemical Society, Washington, D.C. 1991. Those of skill in the art will appreciate that substantially any known drug delivery system is applicable to the conjugates of the present invention.

Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Synthetically modified natural polymers of use in conjugates of the invention include, but are not limited to, alkyl celluloses, hydroxyalkyl celluloses, cellulose ethers, cellulose esters, and nitrocelluloses. Particularly preferred members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

These and the other polymers discussed herein can be readily obtained from commercial sources such as Sigma Chemical Co. (St. Louis, Mo.), Polysciences (Warrenton, Pa.), Aldrich (Milwaukee, Wis.), Fluka (Ronkonkoma, N.Y.), and BioRad (Richmond, Calif.), or else synthesized from monomers obtained from these suppliers using standard techniques.

Representative biodegradable polymers of use in the conjugates of the invention include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

The polymers of use in the invention include “hybrid’ polymers that include water-insoluble materials having within at least a portion of their structure, a bioresorbable molecule. An example of such a polymer is one that includes a water-insoluble copolymer, which has a bioresorbable region, a hydrophilic region and a plurality of crosslinkable functional groups per polymer chain.

For purposes of the present invention, “water-insoluble materials” includes materials that are substantially insoluble in water or water-containing environments. Thus, although certain regions or segments of the copolymer may be hydrophilic or even water-soluble, the polymer molecule, as a whole, does not to any substantial measure dissolve in water.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region may be either hydrophobic or hydrophilic, so long as the copolymer composition as a whole is not rendered water-soluble. Thus, the bioresorbable region is selected based on the preference that the polymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable polymers include, for example, synthetically produced resorbable block copolymers of poly(α-hydroxy-carboxylic acid)/poly(oxyalkylene, (see, Cohn et al., U.S. Pat. No. 4,826,945). These copolymers are not crosslinked and are water-soluble so that the body can excrete the degraded block copolymer compositions. See, Younes et al., J Biomed. Mater. Res. 21: 1301-1316 (1987); and Cohn et al., J Biomed. Mater. Res. 22: 993-1009 (1988).

Presently preferred bioresorbable polymers include one or more components selected from poly(esters), poly(hydroxy acids), poly(lactones), poly(amides), poly(ester-amides), poly(amino acids), poly(anhydrides), poly(orthoesters), poly(carbonates), poly(phosphazines), poly(phosphoesters), poly(thioesters), polysaccharides and mixtures thereof. More preferably still, the bioresorbable polymer includes a poly(hydroxy) acid component. Of the poly(hydroxy) acids, polylactic acid, polyglycolic acid, polycaproic acid, polybutyric acid, polyvaleric acid and copolymers and mixtures thereof are preferred.

In addition to forming fragments that are absorbed in vivo (“bioresorbed”), preferred polymeric coatings for use in the methods of the invention can also form an excretable and/or metabolizable fragment.

Higher order copolymers can also be used in the present invention. For example, Casey et al., U.S. Pat. No. 4,438,253, which issued on Mar. 20, 1984, discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other polymers based on lactic and/or glycolic acids can also be utilized. For example, Spinu, U.S. Pat. No. 5,202,413, which issued on Apr. 13, 1993, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a di-functional compound, such as, a diisocyanate, diacylchloride or dichlorosilane.

Bioresorbable regions of coatings useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene)oxide. Such poly(alkylene)oxides can include, for example, poly(ethylene)oxide, poly(propylene)oxide and mixtures and copolymers thereof.

Polymers that are components of hydrogels are also useful in the present invention. Hydrogels are polymeric materials that are capable of absorbing relatively large quantities of water. Examples of hydrogel forming compounds include, but are not limited to, polyacrylic acids, sodium carboxymethylcellulose, polyvinyl alcohol, polyvinyl pyrrolidine, gelatin, carrageenan and other polysaccharides, hydroxyethylenemethacrylic acid (HEMA), as well as derivatives thereof, and the like. Hydrogels can be produced that are stable, biodegradable and bioresorbable. Moreover, hydrogel compositions can include subunits that exhibit one or more of these properties.

Bio-compatible hydrogel compositions whose integrity can be controlled through crosslinking are known and are presently preferred for use in the methods of the invention. For example, Hubbell et al., U.S. Pat. Nos. 5,410,016, which issued on Apr. 25, 1995 and 5,529,914, which issued on Jun. 25, 1996, disclose water-soluble systems, which are crosslinked block copolymers having a water-soluble central block segment sandwiched between two hydrolytically labile extensions. Such copolymers are further end-capped with photopolymerizable acrylate functionalities. When crosslinked, these systems become hydrogels. The water soluble central block of such copolymers can include poly(ethylene glycol); whereas, the hydrolytically labile extensions can be a poly(α-hydroxy acid), such as polyglycolic acid or polylactic acid. See, Sawhney et al., Macromolecules 26: 581-587 (1993).

In another embodiment, the gel is a thermoreversible gel. Thermoreversible gels including components, such as pluronics, collagen, gelatin, hyalouronic acid, polysaccharides, polyurethane hydrogel, polyurethane-urea hydrogel and combinations thereof are presently preferred.

In yet another exemplary embodiment, the conjugate of the invention includes a component of a liposome. Liposomes can be prepared according to methods known to those skilled in the art, for example, as described in Eppstein et al., U.S. Pat. No. 4,522,811, which issued on Jun. 11, 1985. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its pharmaceutically acceptable salt is then introduced into the container. The container is then swirled by hand to free lipid material from the sides of the container and to disperse lipid aggregates, thereby forming the liposomal suspension.

The above-recited microparticles and methods of preparing the microparticles are offered by way of example and they are not intended to define the scope of microparticles of use in the present invention. It will be apparent to those of skill in the art that an array of microparticles, fabricated by different methods, are of use in the present invention.

The structural formats discussed above in the context of the water-soluble polymers, both straight-chain and branched are generally applicable with respect to the water-insoluble polymers as well. Thus, for example, the cysteine, serine, dilysine, and trilysine branching cores can be functionalized with two water-insoluble polymer moieties. The methods used to produce these species are generally closely analogous to those used to produce the water-soluble polymers.

### Other Modifying Groups

The present invention also provides conjugates analogous to those described above in which the peptide is conjugated to a therapeutic moiety, diagnostic moiety, targeting moiety, toxin moiety or the like via a glycosyl linking group. Each of the above-recited moieties can be a small molecule, natural polymer (e.g., polypeptide) or a synthetic polymer.

In a still further embodiment, the invention provides conjugates that localize selectively in a particular tissue due to the presence of a targeting agent as a component of the conjugate. In an exemplary embodiment, the targeting agent is a protein. Exemplary proteins include transferrin (brain, blood pool), HS-glycoprotein (bone, brain, blood pool), antibodies (brain, tissue with antibody-specific antigen, blood pool), coagulation factors V-XII (damaged tissue, clots, cancer, blood pool), serum proteins, e.g., α-acid glycoprotein, fetuin, α-fetal protein (brain, blood pool), β2-glycoprotein (liver, atherosclerosis plaques, brain, blood pool), G-CSF, GM-CSF, M-CSF, and EPO (immune stimulation, cancers, blood pool, red blood cell overproduction, neuroprotection), albumin (increase in half-life), IL-2 and IFN-α.

In an exemplary targeted conjugate, interferon alpha 2β (IFN-α2β) is conjugated to transferrin via a bifunctional linker that includes a glycosyl linking group at each terminus of the PEG moiety (Scheme 1). For example, one terminus of the PEG linker is functionalized with an intact sialic acid linker that is attached to transferrin and the other is functionalized with an intact C-linked Man linker that is attached to IFN-α2β.

### Biomolecules

In another embodiment, the modified sugar bears a biomolecule. In still further embodiments, the biomolecule is a functional protein, enzyme, antigen, antibody, peptide, nucleic acid (e.g., single nucleotides or nucleosides, oligonucleotides, polynucleotides and single- and higher-stranded nucleic acids), lectin, receptor or a combination thereof.

Preferred biomolecules are essentially non-fluorescent, or emit such a minimal amount of fluorescence that they are inappropriate for use as a fluorescent marker in an assay. Moreover, it is generally preferred to use biomolecules that are not sugars. An exception to this preference is the use of an otherwise naturally occurring sugar that is modified by covalent attachment of another entity (e.g., PEG, biomolecule, therapeutic moiety, diagnostic moiety, etc.). In an exemplary embodiment, a sugar moiety, which is a biomolecule, is conjugated to a linker arm and the sugar-linker arm cassette is subsequently conjugated to a peptide via a method of the invention.

Biomolecules useful in practicing the present invention can be derived from any source. The biomolecules can be isolated from natural sources or they can be produced by synthetic methods. Peptides can be natural peptides or mutated peptides. Mutations can be effected by chemical mutagenesis, site-directed mutagenesis or other means of inducing mutations known to those of skill in the art. Peptides useful in practicing the instant invention include, for example, enzymes, antigens, antibodies and receptors. Antibodies can be either polyclonal or monoclonal; either intact or fragments. The peptides are optionally the products of a program of directed evolution

Both naturally derived and synthetic peptides and nucleic acids are of use in conjunction with the present invention; these molecules can be attached to a sugar residue component or a crosslinking agent by any available reactive group. For example, peptides can be attached through a reactive amine, carboxyl, sulfhydryl, or hydroxyl group. The reactive group can reside at a peptide terminus or at a site internal to the peptide chain. Nucleic acids can be attached through a reactive group on a base (e.g., exocyclic amine) or an available hydroxyl group on a sugar moiety (e.g., 3′- or 5′-hydroxyl). The peptide and nucleic acid chains can be further derivatized at one or more sites to allow for the attachment of appropriate reactive groups onto the chain. See, Chrisey et al. Nucleic Acids Res. 24: 3031-3039 (1996).

In a further embodiment, the biomolecule is selected to direct the peptide modified by the methods of the invention to a specific tissue, thereby enhancing the delivery of the peptide to that tissue relative to the amount of underivatized peptide that is delivered to the tissue. In a still further embodiment, the amount of derivatized peptide delivered to a specific tissue within a selected time period is enhanced by derivatization by at least about 20%, more preferably, at least about 40%, and more preferably still, at least about 100%. Presently, preferred biomolecules for targeting applications include antibodies, hormones and ligands for cell-surface receptors.

In still a further exemplary embodiment, there is provided as conjugate with biotin. Thus, for example, a selectively biotinylated peptide is elaborated by the attachment of an avidin or streptavidin moiety bearing one or more modifying groups.

### Therapeutic Moieties

In another embodiment, the modified sugar includes a therapeutic moiety. Those of skill in the art will appreciate that there is overlap between the category of therapeutic moieties and biomolecules; many biomolecules have therapeutic properties or potential.

The therapeutic moieties can be agents already accepted for clinical use or they can be drugs whose use is experimental, or whose activity or mechanism of action is under investigation. The therapeutic moieties can have a proven action in a given disease state or can be only hypothesized to show desirable action in a given disease state. In another embodiment, the therapeutic moieties are compounds, which are being screened for their ability to interact with a tissue of choice. Therapeutic moieties, which are useful in practicing the instant invention include drugs from a broad range of drug classes having a variety of pharmacological activities. Preferred therapeutic moieties are essentially non-fluorescent, or emit such a minimal amount of fluorescence that they are inappropriate for use as a fluorescent marker in an assay. Moreover, it is generally preferred to use therapeutic moieties that are not sugars. An exception to this preference is the use of a sugar that is modified by covalent attachment of another entity, such as a PEG, biomolecule, therapeutic moiety, diagnostic moiety and the like. In another exemplary embodiment, a therapeutic sugar moiety is conjugated to a linker arm and the sugar-linker arm cassette is subsequently conjugated to a peptide via a method of the invention.

Methods of conjugating therapeutic and diagnostic agents to various other species are well known to those of skill in the art. See, for example Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991.

In an exemplary embodiment, the therapeutic moiety is attached to the modified sugar via a linkage that is cleaved under selected conditions. Exemplary conditions include, but are not limited to, a selected pH (e.g., stomach, intestine, endocytotic vacuole), the presence of an active enzyme (e.g, esterase, reductase, oxidase), light, heat and the like. Many cleavable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989).

Classes of useful therapeutic moieties include, for example, non-steroidal anti-inflammatory drugs (NSAIDS). The NSAIDS can, for example, be selected from the following categories: (e.g., propionic acid derivatives, acetic acid derivatives, fenamic acid derivatives, biphenylcarboxylic acid derivatives and oxicams); steroidal anti-inflammatory drugs including hydrocortisone and the like; antihistaminic drugs (e.g., chlorpheniramine, triprolidine); antitussive drugs (e.g., dextromethorphan, codeine, caramiphen and carbetapentane); antipruritic drugs (e.g., methdilazine and trimeprazine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyramide, quinidine, encamide); β-adrenergic blocker drugs (e.g., metoprolol, acebutolol, betaxolol, labetalol and timolol); cardiotonic drugs (e.g., milrinone, aminone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltiazem, amiodarone, isoxsuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chloroprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazepam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs).

Antimicrobial drugs which are preferred for incorporation into the present composition include, for example, pharmaceutically acceptable salts of β-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isothionate, metronidazole, pentamidine, gentamycin, kanamycin, lineomycin, methacycline, methenamine, minocycline, neomycin, netilmycin, paromomycin, streptomycin, tobramycin, miconazole and amantadine.

Other drug moieties of use in practicing the present invention include antineoplastic drugs (e.g., antiandrogens (e.g., leuprolide or flutamide), cytocidal agents (e.g., adriamycin, doxorubicin, taxol, cyclophosphamide, busulfan, cisplatin, β-2-interferon) anti-estrogens (e.g., tamoxifen), antimetabolites (e.g., fluorouracil, methotrexate, mercaptopurine, thioguanine). Also included within this class are radioisotope-based agents for both diagnosis and therapy, and conjugated toxins, such as ricin, geldanamycin, mytansin, CC-1065, the duocarmycins, Chlicheamycin and related structures and analogues thereof.

The therapeutic moiety can also be a hormone (e.g., medroxyprogesterone, estradiol, leuprolide, megestrol, octreotide or somatostatin); muscle relaxant drugs (e.g., cinnamedrine, cyclobenzaprine, flavoxate, orphenadrine, papaverine, mebeverine, idaverine, ritodrine, diphenoxylate, dantrolene and azumolen); antispasmodic drugs; bone-active drugs (e.g., diphosphonate and phosphonoalkylphosphinate drug compounds); endocrine modulating drugs (e.g., contraceptives (e.g., ethinodiol, ethinyl estradiol, norethindrone, mestranol, desogestrel, medroxyprogesterone), modulators of diabetes (e.g., glyburide or chlorpropamide), anabolics, such as testolactone or stanozolol, androgens (e.g., methyltestosterone, testosterone or fluoxymesterone), antidiuretics (e.g., desmopressin) and calcitonins).

Also of use in the present invention are estrogens (e.g., diethylstilbesterol), glucocorticoids (e.g., triamcinolone, betamethasone, etc.) and progestogens, such as norethindrone, ethynodiol, norethindrone, levonorgestrel; thyroid agents (e.g., liothyronine or levothyroxine) or anti-thyroid agents (e.g., methimazole); antihyperprolactinemic drugs (e.g., cabergoline); hormone suppressors (e.g., danazol or goserelin), oxytocics (e.g., methylergonovine or oxytocin) and prostaglandins, such as mioprostol, alprostadil or dinoprostone, can also be employed.

Other useful modifying groups include immunomodulating drugs (e.g., antihistamines, mast cell stabilizers, such as lodoxamide and/or cromolyn, steroids (e.g., triamcinolone, beclomethazone, cortisone, dexamethasone, prednisolone, methylprednisolone, beclomethasone, or clobetasol), histamine H2 antagonists (e.g., famotidine, cimetidine, ranitidine), immunosuppressants (e.g., azathioprine, cyclosporin), etc. Groups with anti-inflammatory activity, such as sulindac, etodolac, ketoprofen and ketorolac, are also of use. Other drugs of use in conjunction with the present invention will be apparent to those of skill in the art.

### Preparation of Modified Sugars

In general, a covalent bond between the sugar moiety and the modifying group is formed through the use of reactive functional groups, which are typically transformed by the linking process into a new organic functional group or unreactive species. In order to form the bond, the modifying group and the sugar moiety carry complimentary reactive functional groups. The reactive functional group(s), can be located at any position on the sugar moiety.

Reactive groups and classes of reactions useful in practicing the present invention are generally those that are well known in the art of bioconjugate chemistry. Currently favored classes of reactions available with reactive sugar moieties are those, which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

### Reactive Functional Groups

Useful reactive functional groups pendent from a sugar nucleus or modifying group include, but are not limited to:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc; and
(j) epoxides, which can react with, for example, amines and hydroxyl compounds.

The reactive functional groups can be chosen such that they do not participate in, or interfere with, the reactions necessary to assemble the reactive sugar nucleus or modifying group. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### Cross-Linking Groups

Preparation of the modified sugar for use in the methods of the present invention includes attachment of a modifying group to a sugar residue and forming a stable adduct, which is a substrate for a glycosyltransferase. The sugar and modifying group can be coupled by a zero- or higher-order cross-linking agent. Exemplary bifunctional compounds which can be used for attaching modifying groups to carbohydrate moieties include, but are not limited to, bifunctional poly(ethyleneglycols), polyamides, polyethers, polyesters and the like. General approaches for linking carbohydrates to other molecules are known in the literature. See, for example, Lee et al., Biochemistry 28: 1856 (1989); Bhatia et al., Anal. Biochem. 178: 408 (1989); Janda et al., J. Am. Chem. Soc. 112: 8886 (1990) and Bednarski et al., WO 92/18135. In the discussion that follows, the reactive groups are treated as benign on the sugar moiety of the nascent modified sugar. The focus of the discussion is for clarity of illustration. Those of skill in the art will appreciate that the discussion is relevant to reactive groups on the modifying group as well.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase(glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

In addition to the use of site-specific reactive moieties, the present invention contemplates the use of non-specific reactive groups to link the sugar to the modifying group.

Exemplary non-specific cross-linkers include photoactivatable groups, completely inert in the dark, which are converted to reactive species upon absorption of a photon of appropriate energy. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In yet a further embodiment, the linker group is provided with a group that can be cleaved to release the modifying group from the sugar residue. Many cleaveable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups is commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

In the discussion that follows, a number of specific examples of modified sugars that are useful in practicing the present invention are set forth. In the exemplary embodiments, a sialic acid derivative is utilized as the sugar nucleus to which the modifying group is attached. The focus of the discussion on sialic acid derivatives is for clarity of illustration only and should not be construed to limit the scope of the invention. Those of skill in the art will appreciate that a variety of other sugar moieties can be activated and derivatized in a manner analogous to that set forth using sialic acid as an example. For example, numerous methods are available for modifying galactose, glucose, N-acetylgalactosamine and fucose to name a few sugar substrates, which are readily modified by art recognized methods. See, for example, Elhalabi et al., Curr. Med. Chem. 6: 93 (1999) and Schafer et al., J. Org. Chem. 65: 24 (2000).

In an exemplary embodiment, the peptide that is modified by a method of the invention is a glycopeptide that is produced in prokaryotic cells (e.g., E. coli), eukaryotic cells including yeast and mammalian cells (e.g., CHO cells), or in a transgenic animal and thus contains N- and/or O-linked oligosaccharide chains, which are incompletely sialylated. The oligosaccharide chains of the glycopeptide lacking a sialic acid and containing a terminal galactose residue can be glyco-PEG-ylated, glyco-PPG-ylated or otherwise modified with a modified sialic acid.

In Scheme 4, the amino glycoside 1, is treated with the active ester of a protected amino acid (e.g., glycine) derivative, converting the sugar amine residue into the corresponding protected amino acid amide adduct. The adduct is treated with an aldolase to form α-hydroxy carboxylate 2. Compound 2 is converted to the corresponding CMP derivative by the action of CMP-SA synthetase, followed by catalytic hydrogenation of the CMP derivative to produce compound 3. The amine introduced via formation of the glycine adduct is utilized as a locus of PEG or PPG attachment by reacting compound 3 with an activated (m-) PEG or (m-) PPG derivative (e.g., PEG-C(O)NHS, PPG-C(O)NHS), producing 4 or 5, respectively.

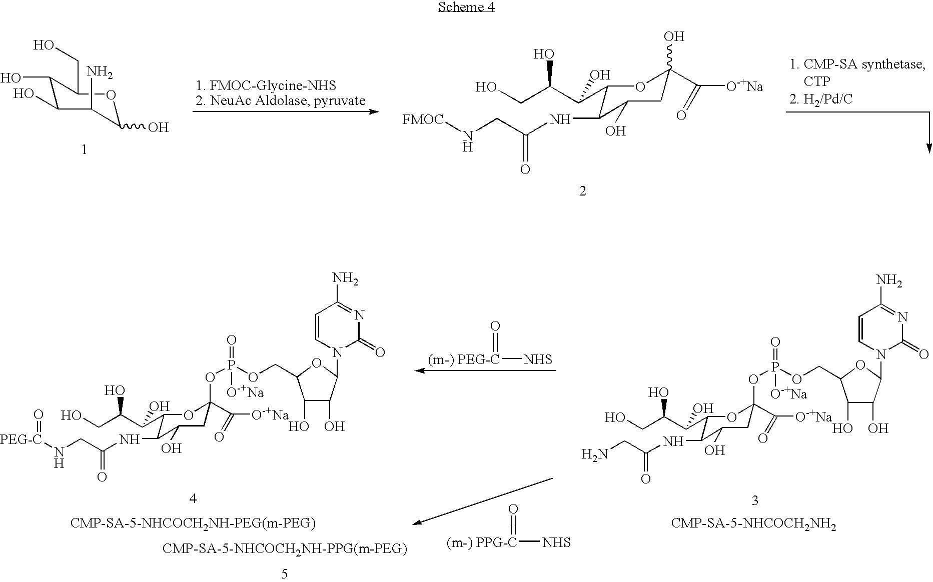

Table 1 sets forth representative examples of sugar monophosphates that are derivatized with a PEG or PPG moiety. Certain of the compounds of Table 2 are prepared by the method of Scheme 4. Other derivatives are prepared by art-recognized methods. See, for example, Keppler et al., Glycobiology 11: 11R (2001); and Charter et al., Glycobiology 10: 1049 (2000)). Other amine reactive PEG and PPG analogues are commercially available, or they can be prepared by methods readily accessible to those of skill in the art.

TABLE 1

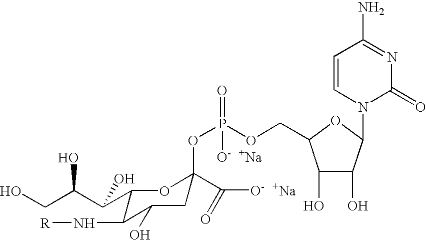

CMP-SA-5-NH—R

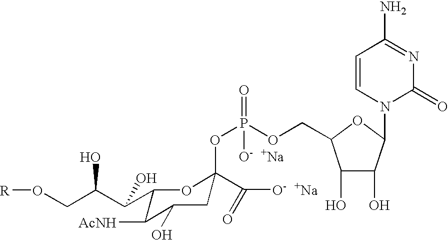

CMP-NeuAc-9-O—R

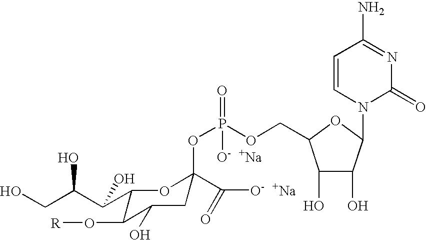

CMP-KDN-5-O—R

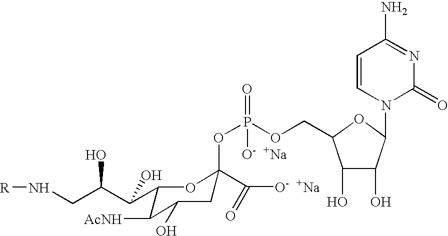

CMP-NeuAc-9-NH—R

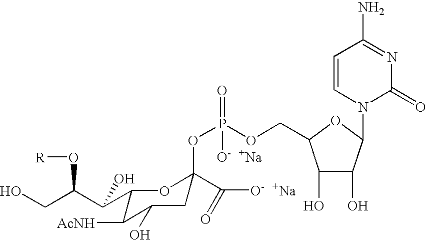

CMP-NeuAc-8-O—R

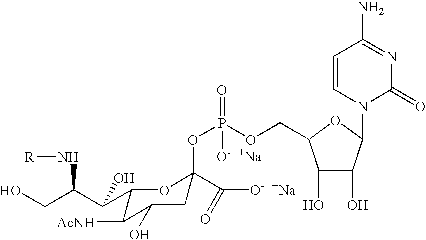

CMP-NeuAc-8-NH—R

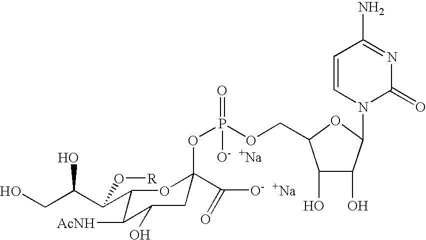

CMP-NeuAc-7-O—R

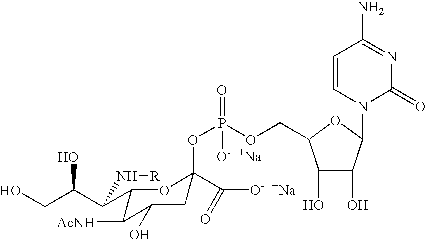

CMP-NeuAc-7-NH—R

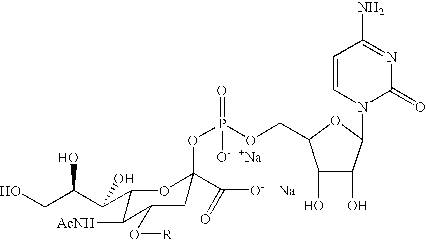

CMP-NeuAc-4-O—R

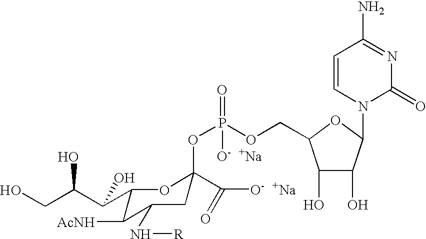

CMP-NeuAc-4-NH—R

The modified sugar phosphates of use in practicing the present invention can be substituted in other positions as well as those set forth above. Presently preferred substitutions of sialic acid are set forth in Formula (VI):

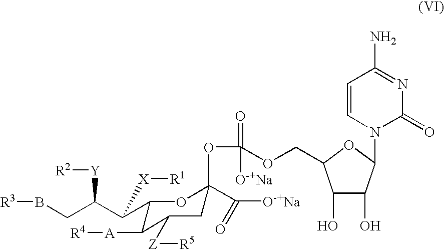

in which X is a linking group, which is preferably selected from —O—, —N(H)—, —S, CH₂—, and —N(R)₂, in which each R is a member independently selected from R¹-R⁵. The symbols Y, Z, A and B each represent a group that is selected from the group set forth above for the identity of X. X, Y, Z, A and B are each independently selected and, therefore, they can be the same or different. The symbols R¹, R², R³, R⁴ and R⁵ represent H, a water-soluble polymer, therapeutic moiety, biomolecule or other moiety. Alternatively, these symbols represent a linker that is bound to a water-soluble polymer, therapeutic moiety, biomolecule or other moiety.

Exemplary moieties attached to the conjugates disclosed herein include, but are not limited to, PEG derivatives (e.g., alkyl-PEG, acyl-PEG, acyl-alkyl-PEG, alkyl-acyl-PEG carbamoyl-PEG, aryl-PEG), PPG derivatives (e.g., alkyl-PPG, acyl-PPG, acyl-alkyl-PPG, alkyl-acyl-PPG carbamoyl-PPG, aryl-PPG), therapeutic moieties, diagnostic moieties, mannose-6-phosphate, heparin, heparan, SLeₓ, mannose, mannose-6-phosphate, Sialyl Lewis X, FGF, VFGF, proteins, chondroitin, keratan, dermatan, albumin, integrins, antennary oligosaccharides, peptides and the like. Methods of conjugating the various modifying groups to a saccharide moiety are readily accessible to those of skill in the art (POLY (ETHYLENE GLYCOL CHEMISTRY: BIOTECHNICAL AND BIOMEDICAL APPLICATIONS, J. Milton Harris, Ed., Plenum Pub. Corp., 1992; POLY(ETHYLENE GLYCOL) CHEMICAL AND BIOLOGICAL APPLICATIONS, J. Milton Harris, Ed., ACS Symposium Series No. 680, American Chemical Society, 1997; Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991).

### Nucleic Acids

In another aspect, the invention provides an isolated nucleic acid encoding a mutant polypeptide of the invention, that includes within its amino acid sequence one or more glycosylation consensus sequence. In one embodiment, the glycosylation consensus sequences are each recognized by an enzyme, such as a glycosyltransferase. In a preferred embodiment, at least one of the glycosylation consensus sequences is not present in the wild-type or parent polypeptide that corresponds to the mutant polypeptide, or is not present at the same site within the wild-type or parent sequence. In one embodiment the nucleic acid of the invention is part of an expression cassette or expression vector. Methods for obtaining the nucleic acid of the invention are known in the art and are described herein below. In another related embodiment, the present invention provides a cell including the nucleic acid of the present invention. In one embodiment, an expression vector including the nucleic acid of the invention is introduced into a cell-type to express the corresponding polypeptide. The polypeptide is then isolated from the cell culture. The cell can be any cell, including bacterial cells, yeast cells, insect cells and mammalian cells.

### Pharmaceutical Compositions

The polypeptide conjugates of the invention have a broad range of pharmaceutical applications. In one aspect, the invention provides a pharmaceutical composition including at least one polypeptide conjugate of the invention as well as a pharmaceutically acceptable diluent, carrier or additive. The pharmaceutical compositions of the invention can contain any polypeptide, including wild-type and mutant polypeptides, which are modified according to the methods of the invention. In an exemplary embodiment, the pharmaceutical composition includes a pharmaceutically acceptable diluent and a covalent conjugate between a non-naturally-occurring, water-soluble polymer and a glycosylated or non-glycosylated polypeptide. Exemplary water-soluble polymers include poly(ethylene glycol) and methoxy-poly(ethylene glycol). Alternatively, the peptide is conjugated to a modifying group other than a poly(ethylene glycol) derivative, such as a therapeutic moiety or a biomolecule. The modifying group is conjugated to the peptide via a glycosyl linking group interposed between and covalently linked to both the peptide or glycopeptide and the modifying group.

Exemplary wild-type or parent polypeptide include bone morphogenetic protein (e.g., BMP-2, BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (ATT, or α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), TNF receptor-IgG Fc region fusion protein (Enbrel™), anti-HER2 monoclonal antibody (Herceptin™), monoclonal antibody to protein F of respiratory syncytial virus (Synagis™), monoclonal antibody to TNF-α (Remicade™), monoclonal antibody to glycoprotein IIb/IIIa (Reopro™), monoclonal antibody to CD20 (Rituxan™), anti-thrombin III (AT III), human chorionic gonadotropin (hCG), alpha-galactosidase (Fabrazyme™), alpha-iduronidase (Aldurazyme™), follicle stimulating hormone (FSH), beta-glucosidase, anti-TNF-alpha monoclonal antibody (MLB 5075), glucagon-like peptide-1 (GLP-1), beta-glucosidase (MLB 5064), alpha-galactosidase A (MLB 5082), fibroblast growth factor (FGF), Factor VII, Factor VIII, Factor IX, prokinetisin and extendin-4, as well as any modified versions (e.g., mutants) thereof.

Pharmaceutical compositions of the invention are useful for the treatment and/or prevention of a variety of diseases and conditions. For example, modified erythropoietin (EPO) may be used for treating general anemia, aplastic anemia, chemo-induced injury (such as injury to bone marrow), chronic renal failure, nephritis, and thalassemia. Modified EPO may further be used for treating neurological disorders such as brain/spine injury, multiple sclerosis, and Alzheimer's disease.

A second example is interferon-α (IFN-α), which may be used for treating AIDS and hepatitis B or C, viral infections caused by a variety of viruses such as human papilloma virus (HBV), coronavirus, human immunodeficiency virus (HIV), herpes simplex virus (HSV), and varicella-zoster virus (VZV), cancers such as hairy cell leukemia, AIDS-related Kaposi's sarcoma, malignant melanoma, follicular non-Hodgkins lymphoma, Philladephia chromosome (Ph)-positive, chronic phase myelogenous leukemia (CML), renal cancer, myeloma, chronic myelogenous leukemia, cancers of the head and neck, bone cancers, as well as cervical dysplasia and disorders of the central nervous system (CNS) such as multiple sclerosis. In addition, IFN-α modified according to the methods of the present invention is useful for treating an assortment of other diseases and conditions such as Sjogren's syndrome (an autoimmune disease), Behcet's disease (an autoimmune inflammatory disease), fibromyalgia (a musculoskeletal pain/fatigue disorder), aphthous ulcer (canker sores), chronic fatigue syndrome, and pulmonary fibrosis.

Another example is interferon-β, which is useful for treating CNS disorders such as multiple sclerosis (either relapsing/remitting or chronic progressive), AIDS and hepatitis B or C, viral infections caused by a variety of viruses such as human papilloma virus (HBV), human immunodeficiency virus (HIV), herpes simplex virus (HSV), and varicella-zoster virus (VZV), otological infections, musculoskeletal infections, as well as cancers including breast cancer, brain cancer, colorectal cancer, non-small cell lung cancer, head and neck cancer, basal cell cancer, cervical dysplasia, melanoma, skin cancer, and liver cancer. IFN-β modified according to the methods of the present invention is also used in treating other diseases and conditions such as transplant rejection (e.g., bone marrow transplant), Huntington's chorea, colitis, brain inflammation, pulmonary fibrosis, macular degeneration, hepatic cirrhosis, and keratoconjunctivitis.

Granulocyte colony stimulating factor (G-CSF) is a further example. G-CSF modified according to the methods of the present invention may be used as an adjunct in chemotherapy for treating cancers, and to prevent or alleviate conditions or complications associated with certain medical procedures, e.g., chemo-induced bone marrow injury; leucopenia (general); chemo-induced febrile neutropenia; neutropenia associated with bone marrow transplants; and severe, chronic neutropenia. Modified G-CSF may also be used for transplantation; peripheral blood cell mobilization; mobilization of peripheral blood progenitor cells for collection in patients who will receive myeloablative or myelosuppressive chemotherapy; and reduction in duration of neutropenia, fever, antibiotic use, hospitalization following induction/consolidation treatment for acute myeloid leukemia (AML). Other condictions or disorders may be treated with modified G-CSF include asthma and allergic rhinitis.

As one additional example, human growth hormone (hGH) modified according to the methods of the present invention may be used to treat growth-related conditions such as dwarfism, short-stature in children and adults, cachexia/muscle wasting, general muscular atrophy, and sex chromosome abnormality (e.g., Turner's Syndrome). Other conditions may be treated using modified hGH include: short-bowel syndrome, lipodystrophy, osteoporosis, uraemaia, burns, female infertility, bone regeneration, general diabetes, type II diabetes, osteo-arthritis, chronic obstructive pulmonary disease (COPD), and insomia. Moreover, modified hGH may also be used to promote various processes, e.g., general tissue regeneration, bone regeneration, and wound healing, or as a vaccine adjunct.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable matrices, such as microspheres (e.g., polylactate polyglycolate), may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered subcutaneously or parenterally, e.g., intravenously. Thus, the invention provides compositions for parenteral administration, which comprise the compound dissolved or suspended in an acceptable carrier, preferably an aqueous carrier, e.g., water, buffered water, saline, PBS and the like. The compositions may also contain detergents such as Tween 20 and Tween 80; stabilizers such as mannitol, sorbitol, sucrose, and trehalose; and preservatives such as EDTA and meta-cresol. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the glycopeptides of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized glycopeptides of the invention.

Targeting mechanisms generally require that the targeting agents be positioned on the surface of the liposome in such a manner that the target moieties are available for interaction with the target, for example, a cell surface receptor. The carbohydrates of the invention may be attached to a lipid molecule before the liposome is formed using methods known to those of skill in the art (e.g., alkylation or acylation of a hydroxyl group present on the carbohydrate with a long chain alkyl halide or with a fatty acid, respectively). Alternatively, the liposome may be fashioned in such a way that a connector portion is first incorporated into the membrane at the time of forming the membrane. The connector portion must have a lipophilic portion, which is firmly embedded and anchored in the membrane. It must also have a reactive portion, which is chemically available on the aqueous surface of the liposome. The reactive portion is selected so that it will be chemically suitable to form a stable chemical bond with the targeting agent or carbohydrate, which is added later. In some cases it is possible to attach the target agent to the connector molecule directly, but in most instances it is more suitable to use a third molecule to act as a chemical bridge, thus linking the connector molecule which is in the membrane with the target agent or carbohydrate which is extended, three dimensionally, off of the vesicle surface.

The polypeptides and conjugates of the invention prepared by the methods of the invention may also find use as diagnostic reagents. For example, labeled compounds can be used to locate areas of inflammation or tumor metastasis in a patient suspected of having an inflammation. For this use, the compounds can, for example, be labeled with ¹²⁵I, ¹⁴C, or tritium.

### V. Methods

Preparation of Polypeptide Conjugates with Carbon- and Nitrogen-Linkages

In another aspect, the invention provides a method for making a polypeptide conjugate of the invention. The method includes the steps of: (a) recombinantly producing the polypeptide, and (b) enzymatically glycosylating the polypeptide at the aromatic amino acid (e.g., tryptophan) residue. In one example glycosylation occurs at a carbon atom or a nitrogen atom that is part of an aromatic amino acid side side. The amino acid, which is the site of glycosylation is preferably located within a glycosylation consensus sequence of the invention.

The conjugates of the invention are formed between polypeptides and diverse species such as water-soluble polymers, therapeutic moieties, diagnostic moieties, targeting moieties and the like. Any polypeptide can be used in the methods of the invention. Exemplary polypeptides include wild-type and mutant polypeptides.

Also provided are conjugates that include two or more peptides linked together through a linker arm, i.e., multifunctional conjugates; at least one peptide being glycosylated at or including a glycosylation consensus sequence of the invention. The multi-functional conjugates of the invention can include two or more copies of the same peptide or a collection of diverse peptides with different structures, and/or properties. In exemplary conjugates according to this embodiment, the linker between the two peptides is attached to at least one of the peptides at an amino acid residue which is part of a glycosylation consensus sequence, either directly or through a glycosyl residue, such as a glycosyl linking group.

In one embodiment, the conjugates of the invention are formed by the enzymatic attachment of a modified sugar to the glycosylated or unglycosylated peptide. The modified sugar is added either directly to an amino acid residue that is part of a glycosylation consensus sequence, or is added to a glycosyl residue attached either directly or indirectly (e.g., through one or more glycosyl residue) to an amino acid residue of a glycosylation consensus sequence.

The modified sugar, when interposed between the peptide (or glycosyl residue) and the modifying group on the sugar becomes what is referred to herein as “a glycosyl linking group.” Using the exquisite selectivity of enzymes, such as glycosyltransferases, the present method provides peptides that bear a desired group at one or more specific locations. Thus, according to the present invention, a modified sugar is attached directly to a selected locus on the peptide chain or, alternatively, the modified sugar is appended onto a carbohydrate moiety of a glycopeptide. Peptides in which modified sugars are bound to both a glycopeptide carbohydrate and directly to an amino acid residue of the peptide backbone are also within the scope of the present invention.

In contrast to known chemical and enzymatic peptide elaboration strategies, the methods of the invention, make it possible to assemble peptides and glycopeptides that have a substantially homogeneous derivatization pattern; the enzymes used in the invention are generally selective for a particular glycosylation consensus sequence. The methods are also practical for large-scale production of modified peptides and glycopeptides. Thus, the methods of the invention provide a practical means for large-scale preparation of glycopeptides having preselected uniform derivatization patterns. The methods are particularly well suited for modification of therapeutic peptides, including but not limited to, glycopeptides that are incompletely glycosylated during production in cell culture (e.g., mammalian cells, insect cells, plant cells, fungal cells, yeast cells, or prokaryotic cells) or transgenic plants or animals.

The methods of the invention also provide conjugates of glycosylated and unglycosylated peptides with increased therapeutic half-life due to, for example, reduced clearance rate, or reduced rate of uptake by the immune or reticuloendothelial system (RES). Moreover, the methods of the invention provide a means for masking antigenic determinants on peptides, thus reducing or eliminating a host immune response against the peptide. Selective attachment of targeting agents to a peptide using an appropriate modified sugar can also be used to target a peptide to a particular tissue or cell surface receptor that is specific for the particular targeting agent. Moreover, there is provided a class of peptides that are specifically modified with a therapeutic moiety conjugated through a glycosyl linking group.

Thus, the invention provides a method of forming a covalent conjugate between a modifying group and a polypeptide. In an exemplary embodiment, the conjugate is formed between a water-soluble polymer, a therapeutic moiety, targeting moiety or a biomolecule, and a glycosylated or non-glycosylated peptide. The polymer, therapeutic moiety or biomolecule is conjugated to the peptide via a glycosyl linking group, which is interposed between, and covalently linked to both the peptide and the modifying group (e.g. water-soluble polymer). The method includes contacting the peptide with a mixture containing a modified sugar and a glycosyltransferase for which the modified sugar is a substrate. The reaction is conducted under conditions appropriate to form a covalent bond between the modified sugar and the peptide. The sugar moiety of the modified sugar is preferably selected from nucleotide sugars, activated sugars and sugars, which are neither nucleotides nor activated.

The acceptor peptide is typically synthesized de novo, or recombinantly expressed in a prokaryotic cell (e.g., bacterial cell, such as E. coli) or in a eukaryotic cell such as a mammalian, yeast, insect, fungal or plant cell. The peptide can be either a full-length protein or a fragment. Moreover, the peptide can be a wild type or mutated peptide. In an exemplary embodiment, the peptide includes a mutation that adds one or more glycosylation sites to the peptide sequence.

In an exemplary embodiment, the peptide is glycosylated at one or more aromatic amino acid (e.g., tryptophan) residues and functionalized with a water-soluble polymer in the following manner: The polypeptide is produced (e.g., expressed in bacterial, insect or mammalian cells) and purified. The amino acid sequence of the polypeptide includes one or more glycosylation consensus sequence of the invention. Subsequently, the polypeptide is glycosylated at one or more of the glycosylation sites. For example, a modified or non-modified mannose residue is added to a tryptophan side chain (e.g., at C-2 or N-1 of the indole ring) using a reagent, that includes a C- or N-mannosyltransferase under conditions sufficient for the mannosyltransferase to transfer a mannosyl residue from a mannosyl-donor [e.g., (GDP)-mannose] onto the polypeptide.

In one embodiment, a modified mannose moiety is added to the aromatic amino acid residue of the glycosylation site using a modified mannosyl (Man*) donor molecule and a mannosyltransferase according to Scheme 5 below.

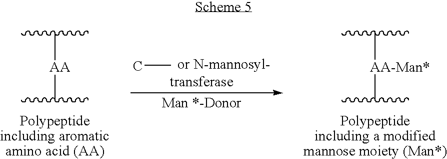

In another embodiment, a mannose moiety is added to the aromatic amino acid and the mannosylated polypeptide is then further glycosylated. In one example a modified glycosyl residue (X*), such as a sialic acid-, Gal-, GalNAc-, Glc or GlcNAc-modifying group cassette, is attached to the mannose residue directly through the use of an appropriate glycosyltransferase, such as a sialyltransferase (e.g., ST6Gal-1), galactosyltransferase or N-acetyl-galactosyltransferase as outlined in Scheme 6, below.

In another example, the modified sugar is added to the mannose residue through one or more additional glycosyl residue (Scheme 6).

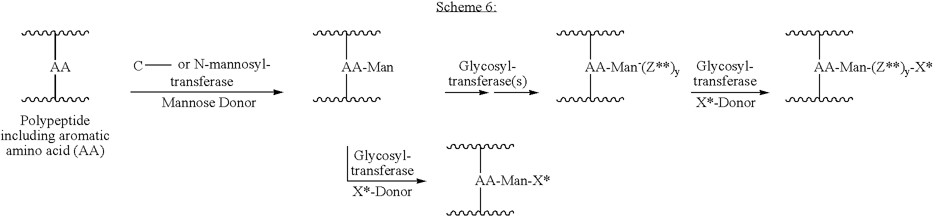

In Scheme 6, Z** represents a glycosyl residue and the integer y is selected from 1 to 20, preferably from 1 to 12. In one embodiment the added glycan is branched. For instance, the mannose residue that is attached to the peptide carries two glycosyl residues that are attached at different positions of the mannose moiety. In an exemplary embodiment, the glycosyl linker is build to resemble glycan structures found in nature for N- and O-linked glycosylation.

In one example the mannosylated peptide is galactosylated using Core-1-GalT-1 and the product is sialylated with a sialic acid-modifying group cassette using ST3GalT1. An exemplary conjugate according to this method has the following exemplary linkages: Try-α-1-GalNAc-β-1,3-Gal-α2,3-Sia*, in which Sia* is the sialic acid linking group that includes one or more modifying group. Scheme 7 summarizes exemplary modified glycan structures.

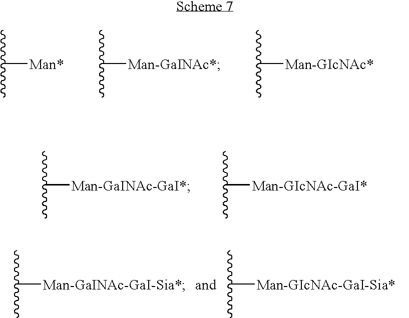

In the methods of the invention, such as that set forth above, using multiple enzymes and saccharyl donors, the individual glycosylation steps may be performed separately, or combined in a “single pot” reaction. For example, in the three enzyme reaction set forth above the GalNAc tranferase, GalT and SiaT and their donors may be combined in a single vessel. Alternatively, the GalNAc reaction can be performed alone and both the GalT and SiaT and the appropriate saccharyl donors added as a single step. Another mode of running the reactions involves adding each enzyme and an appropriate donor sequentially and conducting the reaction in a “single pot” motif. Combinations of each of the methods set forth above are of use in preparing the compounds of the invention.

In the conjugates of the invention, the Sia-modifying group cassette can be linked to the Gal in an α-2,6, or α-2,3 linkage.

The method of the invention also provides for modification of incompletely glycosylated peptides that are produced recombinantly. Many recombinantly produced glycoproteins are incompletely glycosylated, exposing carbohydrate residues that may have undesirable properties, e.g., immunogenicity, recognition by the RES. Employing a modified sugar in a method of the invention, the peptide can be simultaneously further glycosylated and derivatized with, e.g., a water-soluble polymer, therapeutic agent, or the like. The sugar moiety of the modified sugar can be the residue that would properly be conjugated to the acceptor in a fully glycosylated peptide, or another sugar moiety with desirable properties. In another embodiment, unwanted glycosyl residues can be stripped from the polypeptide before targeted glycosylation is performed as described herein.

Peptides modified by the methods of the invention can be synthetic or wild-type peptides or they can be mutated peptides, produced by methods known in the art, such as site-directed mutagenesis. Glycosylation of peptides is typically either N-linked or O-linked. An exemplary N-linkage is the attachment of the modified sugar to the side chain of an asparagine residue. The tripeptide sequences asparagine-X-serine and asparagine-X-threonine, where X is any amino acid except proline, are the recognition sequences for enzymatic attachment of a carbohydrate moiety to the asparagine side chain. Thus, the presence of either of these tripeptide sequences in a polypeptide creates a potential glycosylation site. O-linked glycosylation refers to the attachment of one sugar (e.g., N-acetylgalactosamine, galactose, mannose, GlcNAc, glucose, fucose or xylose) to the hydroxy side chain of a hydroxyamino acid, preferably serine or threonine, although unusual or non-natural amino acids, e.g., 5-hydroxyproline or 5-hydroxylysine may also be used.

Addition of glycosylation sites to a peptide is conveniently accomplished by altering the amino acid sequence such that it contains one or more glycosylation consensus sequences of the invention. The modification may be made by mutation or by full chemical synthesis of the peptide. The peptide amino acid sequence is preferably altered through changes at the DNA level, particularly by mutating the DNA encoding the peptide at preselected bases such that codons are generated that will translate into the desired amino acids. The DNA mutation(s) are preferably made using methods known in the art.

In an exemplary embodiment, the glycosylation site is created by shuffling polynucleotides. Polynucleotides encoding a candidate peptide can be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer, Proc. Natl. Acad. Sci. USA 91:10747-10751 (1994); Stemmer, Nature 370:389-391 (1994); and U.S. Pat. Nos. 5,605,793, 5,837,458, 5,830,721 and 5,811,238.

The present invention also provides means of adding (or removing) one or more selected glycosyl residues to a peptide, after which a modified sugar is conjugated to at least one of the selected glycosyl residues of the peptide. The present embodiment is useful, for example, when it is desired to conjugate the modified sugar to a selected glycosyl residue that is either not present on a peptide or is not present in a desired amount. Thus, prior to coupling a modified sugar to a peptide, the selected glycosyl residue is conjugated to the peptide by enzymatic or chemical coupling. In another embodiment, the glycosylation pattern of a glycopeptide is altered prior to the conjugation of the modified sugar by the removal of a carbohydrate residue from the glycopeptide. See, for example WO 98/31826.

Addition or removal of any carbohydrate moieties present on the glycopeptide is accomplished either chemically or enzymatically. Chemical deglycosylation is preferably brought about by exposure of the polypeptide variant to the compound trifluoromethanesulfonic acid, or an equivalent compound. This treatment results in the cleavage of most or all sugars except the linking sugar (N-acetylglucosamine or N-acetylgalactosamine), while leaving the peptide intact. Chemical deglycosylation is described by Hakimuddin et al., Arch. Biochem. Biophys. 259: 52 (1987) and by Edge et al., Anal Biochem. 118: 131 (1981). Enzymatic cleavage of carbohydrate moieties on polypeptide variants can be achieved by the use of a variety of endo- and exo-glycosidases as described by Thotakura et al., Meth. Enzymol. 138: 350 (1987).

Chemical addition of glycosyl moieties is carried out by any art-recognized method. Enzymatic addition of sugar moieties is preferably achieved using a modification of the methods set forth herein, substituting native glycosyl units for the modified sugars used in the invention. Other methods of adding sugar moieties are disclosed in U.S. Pat. Nos. 5,876,980, 6,030,815, 5,728,554, and 5,922,577.

Exemplary attachment points for selected glycosyl residue include, but are not limited to: (a) consensus sites for glycosylation of an aromatic amino acid and other consensus sites (e.g., O— or N— glycosylation sites), (b) terminal glycosyl moieties that are acceptors for a glycosyltransferase; (c) arginine, asparagine and histidine; (d) free carboxyl groups; (e) free sulfhydryl groups such as those of cysteine; (f) free hydroxyl groups such as those of serine, threonine, or hydroxyproline; (g) aromatic residues such as those of phenylalanine, tyrosine, or tryptophan; or (h) the amide group of glutamine. Exemplary methods of use in the present invention are described in WO 87/05330 published Sep. 11, 1987, and in Aplin and Wriston, CRC CRIT. REV. BIOCHEM., pp. 259-306 (1981).

In one embodiment, the invention provides a method for linking two or more peptides through a linking group. The linking group is of any useful structure and may be selected from straight- and branched-chain structures. Preferably, each terminus of the linker, which is attached to a peptide, includes a modified sugar (i.e., a nascent intact glycosyl linking group). In an exemplary method of the invention, two peptides are linked together via a linker moiety that includes a PEG linker.

### Enzymatic C-2 and N-1 Glycosylation of Tryptophan

Numerous examples are known for proteins, which contain N- or O-linked oligosaccharide residues. More recently, another type of glycosylation has been discovered, which differs fundamentally from N- and O-glycosylation with respect to the protein-carbohydrate linkage. It involves the C-glycosidic attachment of an alpha-mannopyranosyl residue to the C-2 atom of a tryptophan side chain. This modification has been shown to be catalyzed by a microsome associated transferase, which C-mannosylates the first Trp residue in the recognition sequence —WXXW—. The enzyme uses dolichylphosphate mannose as the sugar donor and its activity has been found in mammals, birds, amphibians, and fish. In addition to the wide-spread distribution of the C-mannosyltransferase, its recognition motif, WXXW, has been found in over 300 mammalian proteins. It was therefore suggested that C-mannosylation as a form of protein modification is a common event in vivo, despite the fact that, so far, only few C-mannosylated proteins have been characterized (Hofsteenge et al., J. Biol. Chem. 1999, 274(46): 32786, and cited literature therein).

In addition, it has been discovered (Li J. S. et al., J. Biol. Chem. 2005, 280(46): 38513), that tryptophan cannot only be mannosylated at the C-2 position of the indole ring, but can be mannosylated at the N-1 position as well.

In one embodiment, the present invention provides polypeptide conjugates, that are glycosylated at the side chain of an aromatic amino acid. In a preferred embodiment, the invention provides conjugates wherein the amino acid side chain of a tryptophan or tryptophan derivative is glycosylated as well as methods for forming such conjugates. Of particular interest are conjugates of mutant polypeptides that include suitable consensus sequences, which can be used to direct glycosylation to a particular site within the peptide sequence. In one example, the glycosylation site is a locus for attachment of a glycosyl residue that bears a modifying group.

In one embodiment, microsomal preparations characterized by mannosyltransferase activity can be used to prepare the conjugates of the invention. Alternatively, a desired mannosyltransferase can be isolated (e.g., from an active microsomal preparation) and then be used in the methods of the invention. In another example, the mannosyltransferase is isolated, characterized and then recombinantly produced. The desired enzyme can, for instance, be isolated using activity guided fractionation. Mannosyltransferase activity can be determined using an appropriate assay system. An exemplary assay system involves a test peptide containing a mannosylation consensus sequence of the invention. Mannosylation can be assessed using mass spectroscopical methods, known in the art.

Preparation of Peptide Conjugates with a Thioglycosidic Bond

Thioglycosides, in which the glycosidic oxygen atom has been replaced with a sulfur atom, typically demonstrate increased stability with respect to degradation by glycosidases. This feature of thioglycosides is particularly valuable in therapeutic peptide conjugates of the invention, which demonstrate comparatively short in vivo half-life characteristics. Thioglycoligases or S-glucosyltransferases, which can be useful in preparing thioglycosides, have been described, for example in Jahn M. et al., Angew. Chem. Int. Ed. 2003, 42(3): 352-354 and cited references.

Thus, in another aspect, the invention provides a method of making a peptide conjugate that contains a thioglycosidic linkage. The method includes the steps of: (a) contacting a glycopeptide and a glycosyl linking group and a thioglycoligase, under conditions sufficient for the thioglycoligase to form a covalent bond between said glycopeptide and said glycosyl linking group, wherein a member selected from the glycopeptide and the glycosyl linking group includes a sulfhydryl group.

In an exemplary embodiment, the thioglycoligase is a member selected from a S-glucosyltransferase, and a mutant glycosidase. Exemplary glycosidases include glucosidases, mannosidases, glucuronidases, sialydases, xylosidases and galactosidases. Preferably, the glycosidase is mutated at the acid-base position in the catalytic center of the enzyme. For instance, an amino acid with an acidic side chain (e.g., glutamic acid, aspartic acid) is replaced with an uncharged amino acid. In this example, the acid/base function of the catalytic center is impaired while the catalytic nucleophilic function is retained, thus creating an enzyme which has been described with the term “retaining glycosidase” as the enzyme holds on to the substrate until a secondary nucleophile is added to form a new glycosidic bond. The selection of a suitable glycosidase and a suitable mutation site to produce a desired thioglycoligase is well within the abilities of a skilled person and is described, for instance, in: Salleh H. M. et al., Carbohydrate Research 2006, 341: 49-59 and references cited therein; Muellegger J. et al., Protein Engineering, Design & Selection 2005, 18(1): 33-40 and references cited therein; Jahn M. et al., Angew. Chem. Int. Ed. 2003, 42(3): 352-354 and references cited therein. These references are incorporated herein in their entirety.

In an exemplary embodiment, the thioglycosidic bond of the polypeptide conjugate is formed by contacting a polypeptide that includes a glycosyl residue carrying a sulfhydryl group (e.g., Gal-SH) and a modified sialic acid that includes an activated glycosidic group, such as a dinitrophenyl (DNP) glycosidic group in the presence of a suitable mutated sialydase as outlined in Scheme 5 below:

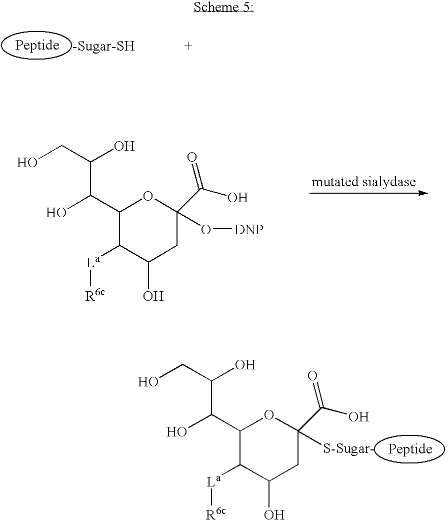

In a related example, the thioglycosidic bond of the polypeptide conjugate is formed by contacting a polypeptide that includes a sugar residue with an activated glycosidic group (such as a DNP galactose) and a thiosugar in the presence of a suitable mutated glycosidase (e.g., galactosidase) as outlined in Scheme 6.

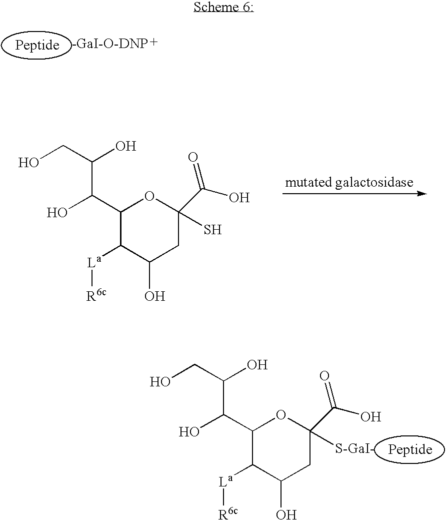

Methods for the preparation of thiosugars and their use in the formation of thioglycosides have been described. See e.g., Zhu X., Schmidt R., Chem. Eur. J. 2004, 10: 875-887; Loureiro Morais L. et al.,

### Acquisition of Peptide Coding Sequences

### General Recombinant Technology

This invention relies on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

Nucleic acid sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Entire genes can also be chemically synthesized. Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of the cloned wild-type peptide genes, polynucleotide encoding mutant peptides, and synthetic oligonucleotides can be verified after cloning using, e.g., the chain termination method for sequencing double-stranded templates of Wallace et al., Gene 16: 21-26 (1981).

### Cloning and Subcloning of a Wild-Type Peptide Coding Sequence

Numerous polynucleotide sequences encoding wild-type peptides have been determined and are available from a commercial supplier, e.g., human growth hormone, e.g., GenBank Accession Nos. NM 000515, NM 002059, NM 022556, NM 022557, NM 022558, NM 022559, NM 022560, NM 022561, and NM 022562.

The rapid progress in the studies of human genome has made possible a cloning approach where a human DNA sequence database can be searched for any gene segment that has a certain percentage of sequence homology to a known nucleotide sequence, such as one encoding a previously identified peptide. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding a peptide can be isolated from a human cDNA or genomic DNA library using standard cloning techniques such as polymerase chain reaction (PCR), where homology-based primers can often be derived from a known nucleic acid sequence encoding a peptide. Most commonly used techniques for this purpose are described in standard texts, e.g., Sambrook and Russell, supra.

cDNA libraries suitable for obtaining a coding sequence for a wild-type peptide may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full-length polynucleotide sequence encoding the wild-type peptide from the cDNA library. A general description of appropriate procedures can be found in Sambrook and Russell, supra.

A similar procedure can be followed to obtain a full length sequence encoding a wild-type peptide, e.g., any one of the GenBank Accession Nos mentioned above, from a human genomic library. Human genomic libraries are commercially available or can be constructed according to various art-recognized methods. In general, to construct a genomic library, the DNA is first extracted from an tissue where a peptide is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a wild-type peptide is obtained.

Upon acquiring a nucleic acid sequence encoding a wild-type peptide, the coding sequence can be subcloned into a vector, for instance, an expression vector, so that a recombinant wild-type peptide can be produced from the resulting construct. Further modifications to the wild-type peptide coding sequence, e.g., nucleotide substitutions, may be subsequently made to alter the characteristics of the molecule.

Introducing Mutations into a Peptide Sequence

From an encoding polynucleotide sequence, the amino acid sequence of a wild-type peptide can be determined. Subsequently, this amino acid sequence may be modified to alter the protein's glycosylation pattern, by introducing additional glycosylation site(s) at various locations in the amino acid sequence.

Several types of protein glycosylation sites are well known in the art. For instance, in eukaryotes, N-linked glycosylation occurs on the asparagine of the consensus sequence Asn-Xₐₐ-Ser/Thr, in which Xₐₐ is any amino acid except proline (Kornfeld et al., Ann Rev Biochem 54:631-664 (1985); Kukuruzinska et al., Proc. Natl. Acad. Sci. USA 84:2145-2149 (1987); Herscovics et al., FASEB J 7:540-550 (1993); and Orlean, Saccharomyces Vol. 3 (1996)). O-linked glycosylation takes place at serine or threonine residues (Tanner et al., Biochim. Biophys. Acta. 906:81-91 (1987); and Hounsell et al., Glycoconj. J. 13:19-26 (1996)). Other glycosylation patterns are formed by linking glycosylphosphatidylinositol to the carboxyl-terminal carboxyl group of the protein (Takeda et al., Trends Biochem. Sci. 20:367-371 (1995); and Udenfriend et al., Ann. Rev. Biochem. 64:593-591 (1995). Based on this knowledge, suitable mutations can thus be introduced into a wild-type peptide sequence to form new glycosylation sites.

Although direct modification of an amino acid residue within a peptide polypeptide sequence may be suitable to introduce a new N-linked or O-linked glycosylation site, more frequently, introduction of a new glycosylation site is accomplished by mutating the polynucleotide sequence encoding a peptide. This can be achieved by using any of known mutagenesis methods, some of which are discussed below. Exemplary modifications to a G-CSF peptide include those illustrated in SEQ ID NO:5-18.

A variety of mutation-generating protocols are established and described in the art. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

### Modification of Nucleic Acids for Preferred Codon Usage in a Host Organism

The polynucleotide sequence encoding a mutant peptide can be further altered to coincide with the preferred codon usage of a particular host. For example, the preferred codon usage of one strain of bacterial cells can be used to derive a polynucleotide that encodes a mutant peptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell. U.S. Pat. No. 5,824,864, for example, provides the frequency of codon usage by highly expressed genes exhibited by dicotyledonous plants and monocotyledonous plants.

At the completion of modification, the mutant peptide coding sequences are verified by sequencing and are then subcloned into an appropriate expression vector for recombinant production in the same manner as the wild-type peptides.

### Expression of the Mutant Peptide

Following sequence verification, the mutant peptide of the present invention can be produced using routine techniques in the field of recombinant genetics, relying on the polynucleotide sequences encoding the polypeptide disclosed herein.

### Expression Systems

To obtain high-level expression of a nucleic acid encoding a mutant peptide of the present invention, one typically subclones a polynucleotide encoding the mutant peptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the wild-type or mutant peptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the mutant peptide in host cells. A typical expression cassette thus contains a promoter operably linked to the nucleic acid sequence encoding the mutant peptide and signals required for efficient polyadenylation of the transcript, ribosome binding sites, and translation termination. The nucleic acid sequence encoding the peptide is typically linked to a cleavable signal peptide sequence to promote secretion of the peptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322-based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

In some exemplary embodiments the expression vector is chosen from pCWin1, pCWin2, pCWin2/MBP, pCWin2-MBP-SBD (pMS₃₉), and pCWin2-MBP-MCS-SBD (pMXS₃₉) as disclosed in co-owned U.S. patent application filed Apr. 9, 2004 which is incorporated herein by reference.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the mutant peptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

When periplasmic expression of a recombinant protein (e.g., a hgh mutant of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that various conservative substitutions can be made to any wild-type or mutant peptide or its coding sequence while still retaining the biological activity of the peptide. Moreover, modifications of a polynucleotide coding sequence may also be made to accommodate preferred codon usage in a particular expression host without altering the resulting amino acid sequence.

### Transfection Methods

Standard transfection methods are used to produce bacterial, mammalian, yeast or insect cell lines that express large quantities of the mutant peptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the mutant peptide.

### Detection of Expression of Mutant Peptide in Host Cells

After the expression vector is introduced into appropriate host cells, the transfected cells are cultured under conditions favoring expression of the mutant peptide. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a mutant peptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a mutant peptide of the present invention, such as a polypeptide having the amino acid sequence of SEQ ID NO:1-7, (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the mutant peptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6: 511-519 (1976). More detailed descriptions of preparing antibody against the mutant peptide of the present invention and conducting immunological assays detecting the mutant peptide are provided in a later section.

### Purification of Recombinantly Produced Mutant Peptide

Once the expression of a recombinant mutant peptide in transfected host cells is confirmed, the host cells are then cultured in an appropriate scale for the purpose of purifying the recombinant polypeptide.

1. Purification from Bacteria

When the mutant peptides of the present invention are produced recombinantly by transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, NY). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant peptide from bacterial inclusion body, see, e.g., Patra et al., Protein Expression and Purification 18: 182-190 (2000).

Alternatively, it is possible to purify recombinant polypeptides, e.g., a mutant peptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 2. Standard Protein Separation Techniques for Purification

When a recombinant polypeptide, e.g., the mutant peptide of the present invention, is expressed in host cells in a soluble form, its purification can follow the standard protein purification procedure described below.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a mutant peptide of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Ultrafiltration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a mutant peptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as the mutant peptide of the present invention) can also be separated from other proteins on the basis of their size, net surface charge, hydrophobicity, or affinity for ligands. In addition, antibodies raised against peptide can be conjugated to column matrices and the peptide immunopurified. All of these methods are well known in the art.

It will be apparent to one of skill that chromatographic techniques can be performed at any scale and using equipment from many different manufacturers (e.g., Pharmacia Biotech).

### Immunoassays for Detection of Mutant Peptide Expression

To confirm the production of a recombinant mutant peptide, immunological assays may be useful to detect in a sample the expression of the polypeptide. Immunological assays are also useful for quantifying the expression level of the recombinant hormone. Antibodies against a mutant peptide are necessary for carrying out these immunological assays.

Production of Antibodies against Mutant Peptide

Methods for producing polyclonal and monoclonal antibodies that react specifically with an immunogen of interest are known to those of skill in the art (see, e.g., Coligan, Current Protocols in Immunology Wiley/Greene, N.Y., 1991; Harlow and Lane, Antibodies: A Laboratory Manual Cold Spring Harbor Press, N.Y., 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256: 495-497, 1975). Such techniques include antibody preparation by selection of antibodies from libraries of recombinant antibodies in phage or similar vectors (see, Huse et al., Science 246: 1275-1281, 1989; and Ward et al., Nature 341: 544-546, 1989).

In order to produce antisera containing antibodies with desired specificity, the polypeptide of interest (e.g., a mutant peptide of the present invention) or an antigenic fragment thereof can be used to immunize suitable animals, e.g., mice, rabbits, or primates. A standard adjuvant, such as Freund's adjuvant, can be used in accordance with a standard immunization protocol. Alternatively, a synthetic antigenic peptide derived from that particular polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be performed subsequently, see, Harlow and Lane, supra, and the general descriptions of protein purification provided above.

Monoclonal antibodies are obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. The general principles and methods of recombinant polypeptide production discussed above are applicable for antibody production by recombinant methods.

When desired, antibodies capable of specifically recognizing a mutant peptide of the present invention can be tested for their cross-reactivity against the wild-type peptide and thus distinguished from the antibodies against the wild-type protein. For instance, antisera obtained from an animal immunized with a mutant peptide can be run through a column on which a wild-type peptide is immobilized. The portion of the antisera that passes through the column recognizes only the mutant peptide and not the wild-type peptide. Similarly, monoclonal antibodies against a mutant peptide can also be screened for their exclusivity in recognizing only the mutant but not the wild-type peptide.

Polyclonal or monoclonal antibodies that specifically recognize only the mutant peptide of the present invention but not the wild-type peptide are useful for isolating the mutant protein from the wild-type protein, for example, by incubating a sample with a mutant peptide-specific polyclonal or monoclonal antibody immobilized on a solid support.

### Immunoassays for Detecting Mutant Peptide Expression

Once antibodies specific for a mutant peptide of the present invention are available, the amount of the polypeptide in a sample, e.g., a cell lysate, can be measured by a variety of immunoassay methods providing qualitative and quantitative results to a skilled artisan. For a review of immunological and immunoassay procedures in general see, e.g., Stites, supra; U.S. Pat. Nos. 4,366,241; 4,376,110; 4,517,288; and 4,837,168.

### Labeling in Immunoassays

Immunoassays often utilize a labeling agent to specifically bind to and label the binding complex formed by the antibody and the target protein. The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and calorimetric labels such as colloidal gold or colored glass or plastic (e.g., polystyrene, polypropylene, latex, etc.) beads.

In some cases, the labeling agent is a second antibody bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al., J. Immunol., 135: 2589-2542 (1985)).

### Immunoassay Formats

Immunoassays for detecting a target protein of interest (e.g., a mutant human growth hormone) from samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second or third antibody bearing a label, as described above.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized.

In some cases, western blot (immunoblot) analysis is used to detect and quantify the presence of a mutant peptide in the samples. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against a mutant peptide.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5: 34-41 (1986)).

### Conjugation of Modified Sugars to Peptides

The modified sugars are conjugated to a glycosylated or non-glycosylated peptide using an appropriate enzyme to mediate the conjugation. Preferably, the concentrations of the modified donor sugar(s), enzyme(s) and acceptor peptide(s) are selected such that glycosylation proceeds until the acceptor is consumed. The considerations discussed below, while set forth in the context of a sialyltransferase, are generally applicable to other glycosyltransferase reactions.

A number of methods of using glycosyltransferases to synthesize desired oligosaccharide structures are known and are generally applicable to the instant invention. Exemplary methods are described, for instance, WO 96/32491, Ito et al., Pure Appl. Chem. 65: 753 (1993), and U.S. Pat. Nos. 5,352,670, 5,374,541, and 5,545,553.

The present invention is practiced using a single glycosyltransferase or a combination of glycosyltransferases. For example, one can use a combination of a sialyltransferase and a galactosyltransferase. In those embodiments using more than one enzyme, the enzymes and substrates are preferably combined in an initial reaction mixture, or the enzymes and reagents for a second enzymatic reaction are added to the reaction medium once the first enzymatic reaction is complete or nearly complete. By conducting two enzymatic reactions in sequence in a single vessel, overall yields are improved over procedures in which an intermediate species is isolated. Moreover, cleanup and disposal of extra solvents and by-products is reduced.

In another embodiment, each of the first and second enzyme is a glycosyltransferase. In another embodiment, one enzyme is an endoglycosidase. In an additional embodiment, more than two enzymes are used to assemble the modified glycoprotein of the invention. The enzymes are used to alter a saccharide structure on the peptide at any point either before or after the addition of the modified sugar to the peptide.

The O-linked glycosyl moieties of the conjugates of the invention are generally originate with a GalNAc moiety that is attached to the peptide. Any member of the family of GalNAc transferases can be used to bind a GalNAc moiety to the peptide (Hassan H, Bennett EP, Mandel U, Hollingsworth Mass., and Clausen H (2000). Control of Mucin-Type O-Glycosylation: O-Glycan Occupancy is Directed by Substrate Specificities of Polypeptide GalNAc-Transferases. (Eds. Ernst, Hart, and Sinay). Wiley-VCH chapter “Carbohydrates in Chemistry and Biology—a Comprehension Handbook”, 273-292). The GalNAc moiety itself can be the intact glycosyl linker. Alternatively, the saccharyl residue is built out using one more enzyme and one or more appropriate glycosyl substrate for the enzyme, the modified sugar being added to the built out glycosyl moiety.

In another embodiment, the method makes use of one or more exo- or endoglycosidase. The glycosidase is typically a mutant, which is engineered to form glycosyl bonds rather than cleave them. The mutant glycanase typically includes a substitution of an amino acid residue for an active site acidic amino acid residue. For example, when the endoglycanase is endo-H, the substituted active site residues will typically be Asp at position 130, Glu at position 132 or a combination thereof. The amino acids are generally replaced with serine, alanine, asparagine, or glutamine.

The mutant enzyme catalyzes the reaction, usually by a synthesis step that is analogous to the reverse reaction of the endoglycanase hydrolysis step. In these embodiments, the glycosyl donor molecule (e.g., a desired oligo- or mono-saccharide structure) contains a leaving group and the reaction proceeds with the addition of the donor molecule to a GlcNAc residue on the protein. For example, the leaving group can be a halogen, such as fluoride. In other embodiments, the leaving group is a Asn, or a Asn-peptide moiety. In yet further embodiments, the GlcNAc residue on the glycosyl donor molecule is modified. For example, the GlcNAc residue may comprise a 1,2 oxazoline moiety.

In another embodiment, each of the enzymes utilized to produce a conjugate of the invention are present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

The temperature at which an above process is carried out can range from just above freezing to the temperature at which the most sensitive enzyme denatures. Preferred temperature ranges are about 0° C. to about 55° C., and more preferably about 20° C. to about 30° C. In another exemplary embodiment, one or more components of the present method are conducted at an elevated temperature using a thermophilic enzyme.

The reaction mixture is maintained for a period of time sufficient for the acceptor to be glycosylated, thereby forming the desired conjugate. Some of the conjugate can often be detected after a few hours, with recoverable amounts usually being obtained within 24 hours or less. Those of skill in the art understand that the rate of reaction is dependent on a number of variable factors (e.g, enzyme concentration, donor concentration, acceptor concentration, temperature, solvent volume), which are optimized for a selected system.

The present invention also provides for the industrial-scale production of modified peptides. As used herein, an industrial scale generally produces at least about 250 mg, preferably at least about 500 mg, and more preferably at least about 1 gram of finished, purified conjugate, preferably after a single reaction cycle, i.e., the conjugate is not a combination the reaction products from identical, consecutively iterated synthesis cycles.

In the discussion that follows, the invention is exemplified by the conjugation of modified sialic acid moieties to a glycosylated peptide. The exemplary modified sialic acid is labeled with (m-) PEG. The focus of the following discussion on the use of PEG-modified sialic acid and glycosylated peptides is for clarity of illustration and is not intended to imply that the invention is limited to the conjugation of these two partners. One of skill understands that the discussion is generally applicable to the additions of modified glycosyl moieties other than sialic acid. Moreover, the discussion is equally applicable to the modification of a glycosyl unit with agents other than PEG including other water-soluble polymers, therapeutic moieties, and biomolecules.

An enzymatic approach can be used for the selective introduction of (m-) PEG-ylated or (m-) PPG-ylated carbohydrates onto a peptide or glycopeptide. The method utilizes modified sugars containing PEG, PPG, or a masked reactive functional group, and is combined with the appropriate glycosyltransferase or glycosynthase. By selecting the glycosyltransferase that will make the desired carbohydrate linkage and utilizing the modified sugar as the donor substrate, the PEG or PPG can be introduced directly onto the peptide backbone, onto existing sugar residues of a glycopeptide or onto sugar residues that have been added to a peptide.

An acceptor for the sialyltransferase is present on the peptide to be modified by the methods of the present invention either as a naturally occurring structure or one placed there recombinantly, enzymatically or chemically. Suitable acceptors, include, for example, galactosyl acceptors such as GalNAc, Galβ1,4GlcNAc, Galβ1,4GalNAc, Galβ1,3GalNAc, lacto-N-tetraose, Galβ1,3GlcNAc, Galβ1,3Ara, Galβ1,6GlcNAc, Galβ1,4Glc (lactose), and other acceptors known to those of skill in the art (see, e.g., Paulson et al., J. Biol. Chem. 253: 5617-5624 (1978)).

In one embodiment, an acceptor for the sialyltransferase is present on the glycopeptide to be modified upon in vivo synthesis of the glycopeptide. Such glycopeptides can be sialylated using the claimed methods without prior modification of the glycosylation pattern of the glycopeptide. Alternatively, the methods of the invention can be used to sialylate a peptide that does not include a suitable acceptor; one first modifies the peptide to include an acceptor by methods known to those of skill in the art. In an exemplary embodiment, a GalNAc residue is added to an O-linked glycosylation site by the action of a GalNAc transferase. Hassan H, Bennett E P, Mandel U, Hollingsworth M A, and Clausen H (2000). Control of Mucin-Type O-Glycosylation: O-Glycan Occupancy is Directed by Substrate Specificities of Polypeptide GalNAc-Transferases. (Eds. Ernst, Hart, and Sinay). Wiley-VCH chapter “Carbohydrates in Chemistry and Biology—a Comprehension Handbook”, 273-292.

In an exemplary embodiment, the galactosyl acceptor is assembled by attaching a galactose residue to an appropriate acceptor linked to the peptide, e.g., a GalNAc. The method includes incubating the peptide to be modified with a reaction mixture that contains a suitable amount of a galactosyltransferase (e.g., Galβ1,3 or Galβ1,4), and a suitable galactosyl donor (e.g., UDP-galactose). The reaction is allowed to proceed substantially to completion or, alternatively, the reaction is terminated when a preselected amount of the galactose residue is added. Other methods of assembling a selected saccharide acceptor will be apparent to those of skill in the art.

In yet another embodiment, glycopeptide-linked oligosaccharides are first “trimmed,” either in whole or in part, to expose either an acceptor for the sialyltransferase or a moiety to which one or more appropriate residues can be added to obtain a suitable acceptor. Enzymes such as glycosyltransferases and endoglycosidases (see, for example U.S. Pat. No. 5,716,812) are useful for the attaching and trimming reactions.

In the discussion that follows, the method of the invention is exemplified by the use of modified sugars having a water-soluble polymer attached thereto. The focus of the discussion is for clarity of illustration. Those of skill will appreciate that the discussion is equally relevant to those embodiments in which the modified sugar bears a therapeutic moiety, biomolecule or the like.

In an exemplary embodiment, an O-linked carbohydrate residue is “trimmed” prior to the addition of the modified sugar. For example a GalNAc-Gal residue is trimmed back to GalNAc. A modified sugar bearing a water-soluble polymer is conjugated to one or more of the sugar residues exposed by the “trimming.” In one example, a glycopeptide is “trimmed” and a water-soluble polymer is added to the resulting O-side chain amino acid or glycopeptide glycan via a saccharyl moiety, e.g., Sia, Gal or GalNAc moiety conjugated to the water-soluble polymer. The modified saccharyl moiety is attached to an acceptor site on the “trimmed” glycopeptide. Alternatively, an unmodified saccharyl moiety, e.g., Gal can be added the terminus of the O-linked glycan.

In another exemplary embodiment, a water-soluble polymer is added to a GalNAc residue via a modified sugar having a galactose residue. Alternatively, an unmodified Gal can be added to the terminal GalNAc residue.

In yet a further example, a water-soluble polymer is added onto a Gal residue using a modified sialic acid.

In another exemplary embodiment, an O-linked glycosyl residue is “trimmed back” to the GalNAc attached to the amino acid. In one example, a water-soluble polymer is added via a Gal modified with the polymer. Alternatively, an unmodified Gal is added to the GalNAc, followed by a Gal with an attached water-soluble polymer. In yet another embodiment, one or more unmodified Gal residue is added to the GalNAc, followed by a sialic acid moiety modified with a water-soluble polymer.

The exemplary embodiments discussed above provide an illustration of the power of the methods set forth herein. Using the methods of the invention, it is possible to “trim back” and build up a carbohydrate residue of substantially any desired structure. The modified sugar can be added to the termini of the carbohydrate moiety as set forth above, or it can be intermediate between the peptide core and the terminus of the carbohydrate.

In an exemplary embodiment, the water-soluble polymer is added to a terminal Gal residue using a polymer modified sialic acid. An appropriate sialyltransferase is used to add a modified sialic acid. The approach is summarized in Scheme 5.

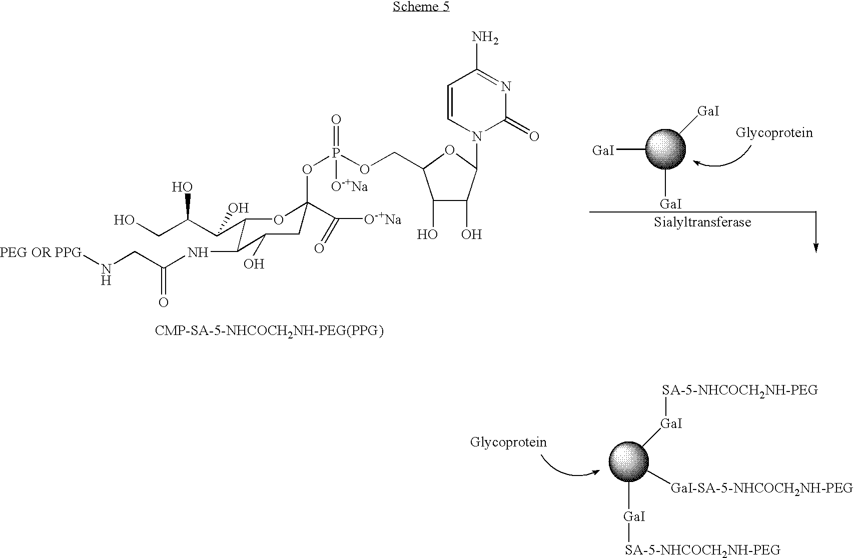

In yet a further approach, summarized in Scheme 6, a masked reactive functionality is present on the sialic acid. The masked reactive group is preferably unaffected by the conditions used to attach the modified sialic acid to the peptide. After the covalent attachment of the modified sialic acid to the peptide, the mask is removed and the peptide is conjugated with an agent such as PEG, PPG, a therapeutic moiety, biomolecule or other agent. The agent is conjugated to the peptide in a specific manner by its reaction with the unmasked reactive group on the modified sugar residue.

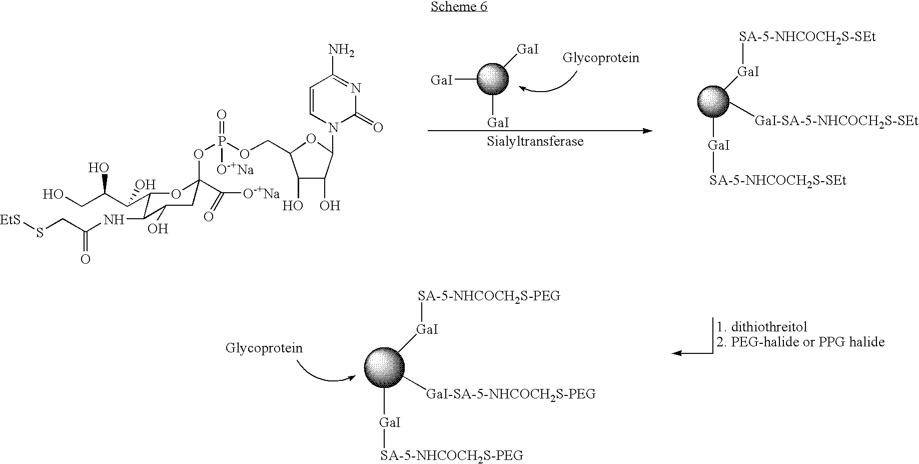

Any modified sugar can be used with its appropriate glycosyltransferase, depending on the terminal sugars of the oligosaccharide side chains of the glycopeptide (Table 3). As discussed above, the terminal sugar of the glycopeptide required for introduction of the PEG-ylated or PPGylated structure can be introduced naturally during expression or it can be produced post expression using the appropriate glycosidase(s), glycosyltransferase(s) or mix of glycosidase(s) and glycosyltransferase(s).

TABLE 3

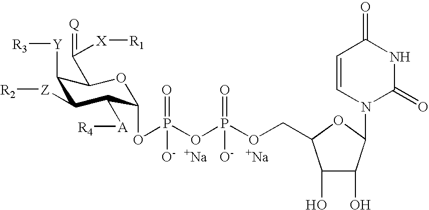

UDP-galactose-derivatives

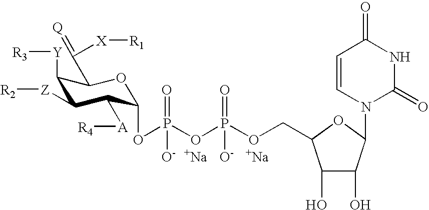

UDP-galactosamine-derivatives
(when A = NH, R₄ may be acetyl)

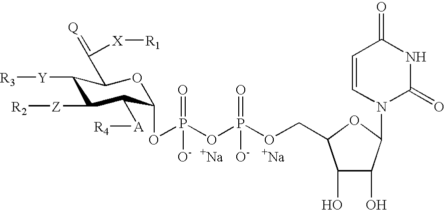

UDP-Glucose-derivatives

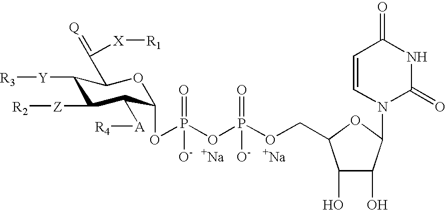

P-Glucosamine-derivatives
(when A = Nh, R₄ may be acetyl)

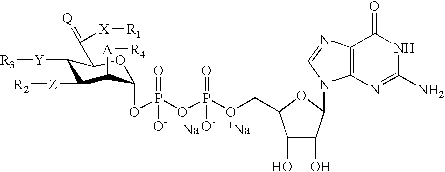

GDP-Mannose-derivatives

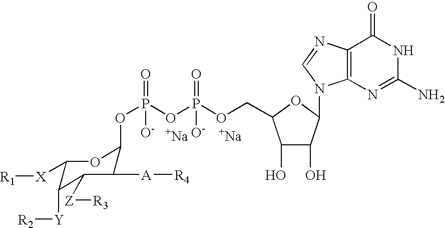

GDP-fucose-derivatives
X = O, NH, S, CH₂, N—(R₁₋₅)₂.
Y = X; Z = X; A = X; B = X.
Q = H₂, O, S, NH, N—R.
R, R₁₋₄ = H, Linker-M, M.
M = Ligand of interest
Ligand of interest = acyl-PEG, acyl-PPG, alkyl-PEG, acyl-alkyl-PEG, acyl-alkyl-PEG, carbamoyl-PEG, carbamoyl-PPG, PEG, PPG, acyl-aryl-PEG, acyl-aryl-PPG, aryl-PEG, aryl-PPG, Mannose-₆-phosphate, heparin, heparan, SLex, Mannose, FGF, VFGF, protein, chondroitin, keratan, dermatan, albumin, integrins, peptides, etc.

In an alternative embodiment, the modified sugar is added directly to the peptide backbone using a glycosyltransferase known to transfer sugar residues to the O-linked glycosylation site on the peptide backbone. This exemplary embodiment is set forth in Scheme 7. Exemplary glycosyltransferases useful in practicing the present invention include, but are not limited to, GalNAc transferases (GalNAc T1-20), GlcNAc transferases, fucosyltransferases, glucosyltransferases, xylosyltransferases, mannosyltransferases and the like. Use of this approach allows the direct addition of modified sugars onto peptides that lack any carbohydrates or, alternatively, onto existing glycopeptides. In both cases, the addition of the modified sugar occurs at specific positions on the peptide backbone as defined by the substrate specificity of the glycosyltransferase and not in a random manner as occurs during modification of a protein's peptide backbone using chemical methods. An array of agents can be introduced into proteins or glycopeptides that lack the glycosyltransferase substrate peptide sequence by engineering the appropriate amino acid sequence into the polypeptide chain.

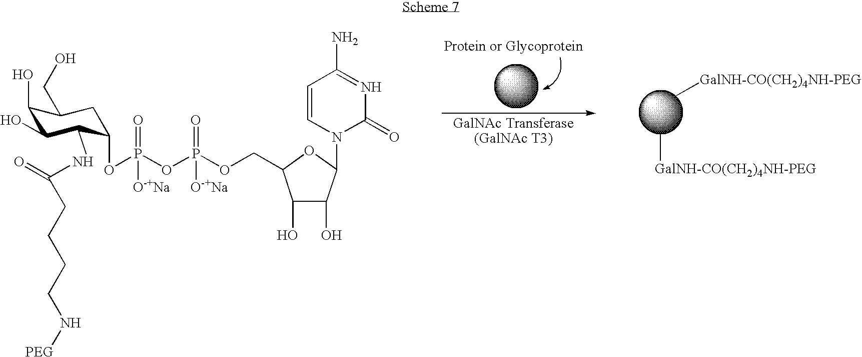

In each of the exemplary embodiments set forth above, one or more additional chemical or enzymatic modification steps can be utilized following the conjugation of the modified sugar to the peptide. In an exemplary embodiment, an enzyme (e.g., fucosyltransferase) is used to append a glycosyl unit (e.g., fucose) onto the terminal modified sugar attached to the peptide. In another example, an enzymatic reaction is utilized to “cap” (e.g., sialylate) sites to which the modified sugar failed to conjugate. Alternatively, a chemical reaction is utilized to alter the structure of the conjugated modified sugar. For example, the conjugated modified sugar is reacted with agents that stabilize or destabilize its linkage with the peptide component to which the modified sugar is attached. In another example, a component of the modified sugar is deprotected following its conjugation to the peptide. One of skill will appreciate that there is an array of enzymatic and chemical procedures that are useful in the methods of the invention at a stage after the modified sugar is conjugated to the peptide. Further elaboration of the modified sugar-peptide conjugate is within the scope of the invention.

In another exemplary embodiment, the glycopeptide is conjugated to a targeting agent, e.g., transferrin (to deliver the peptide across the blood-brain barrier, and to endosomes), carnitine (to deliver the peptide to muscle cells; see, for example, LeBorgne et al., Biochem. Pharmacol. 59: 1357-63 (2000), and phosphonates, e.g., bisphosphonate (to target the peptide to bone and other calciferous tissues; see, for example, Modern Drug Discovery, August 2002, page 10). Other agents useful for targeting are apparent to those of skill in the art. For example, glucose, glutamine and IGF are also useful to target muscle.

The targeting moiety and therapeutic peptide are conjugated by any method discussed herein or otherwise known in the art. Those of skill will appreciate that peptides in addition to those set forth above can also be derivatized as set forth herein. Exemplary peptides are set forth in the Appendix attached to copending, commonly owned U.S. Provisional Patent Application No. 60/328,523 filed Oct. 10, 2001.

In an exemplary embodiment, the targeting agent and the therapeutic peptide are coupled via a linker moiety. In this embodiment, at least one of the therapeutic peptide or the targeting agent is coupled to the linker moiety via an intact glycosyl linking group according to a method of the invention. In an exemplary embodiment, the linker moiety includes a poly(ether) such as poly(ethylene glycol). In another exemplary embodiment, the linker moiety includes at least one bond that is degraded in vivo, releasing the therapeutic peptide from the targeting agent, following delivery of the conjugate to the targeted tissue or region of the body.

In yet another exemplary embodiment, the in vivo distribution of the therapeutic moiety is altered via altering a glycoform on the therapeutic moiety without conjugating the therapeutic peptide to a targeting moiety. For example, the therapeutic peptide can be shunted away from uptake by the reticuloendothelial system by capping a terminal galactose moiety of a glycosyl group with sialic acid (or a derivative thereof).

### Enzymes

### Glycosyltransferases

Glycosyltransferases catalyze the addition of activated sugars (donor NDP-sugars), in a step-wise fashion, to a protein, glycopeptide, lipid or glycolipid or to the non-reducing end of a growing oligosaccharide. N-linked glycopeptides are synthesized via a transferase and a lipid-linked oligosaccharide donor Dol-PP-NAG₂Glc₃Man₉ in an en block transfer followed by trimming of the core. In this case the nature of the “core” saccharide is somewhat different from subsequent attachments. A very large number of glycosyltransferases are known in the art.

The glycosyltransferase to be used in the present invention may be any as long as it can utilize the modified sugar as a sugar donor. Examples of such enzymes include Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like.

For enzymatic saccharide syntheses that involve glycosyltransferase reactions, glycosyltransferase can be cloned, or isolated from any source. Many cloned glycosyltransferases are known, as are their polynucleotide sequences. See, e.g., “The WWW Guide To Cloned Glycosyltransferases,” (http://www.vei.co.uk/TGN/gt_guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligosaccharyltransferases. Suitable glycosyltransferases include those obtained from eukaryotes, as well as from prokaryotes.

DNA encoding glycosyltransferases may be obtained by chemical synthesis, by screening reverse transcripts of mRNA from appropriate cells or cell line cultures, by screening genomic libraries from appropriate cells, or by combinations of these procedures. Screening of mRNA or genomic DNA may be carried out with oligonucleotide probes generated from the glycosyltransferases gene sequence. Probes may be labeled with a detectable group such as a fluorescent group, a radioactive atom or a chemiluminescent group in accordance with known procedures and used in conventional hybridization assays. In the alternative, glycosyltransferases gene sequences may be obtained by use of the polymerase chain reaction (PCR) procedure, with the PCR oligonucleotide primers being produced from the glycosyltransferases gene sequence. See, U.S. Pat. No. 4,683,195 to Mullis et al. and U.S. Pat. No. 4,683,202 to Mullis.

The glycosyltransferase may be synthesized in host cells transformed with vectors containing DNA encoding the glycosyltransferases enzyme. Vectors are used either to amplify DNA encoding the glycosyltransferases enzyme and/or to express DNA which encodes the glycosyltransferases enzyme. An expression vector is a replicable DNA construct in which a DNA sequence encoding the glycosyltransferases enzyme is operably linked to suitable control sequences capable of effecting the expression of the glycosyltransferases enzyme in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences which control the termination of transcription and translation. Amplification vectors do not require expression control domains. All that is needed is the ability to replicate in a host, usually conferred by an origin of replication, and a selection gene to facilitate recognition of transformants.

In an exemplary embodiment, the invention utilizes a prokaryotic enzyme. Such glycosyltransferases include enzymes involved in synthesis of lipooligosaccharides (LOS), which are produced by many gram negative bacteria (Preston et al., Critical Reviews in Microbiology 23(3): 139-180 (1996)). Such enzymes include, but are not limited to, the proteins of the rfa operons of species such as E. coli and Salmonella typhimurium, which include a β1,6 galactosyltransferase and a β1,3 galactosyltransferase (see, e.g., EMBL Accession Nos. M80599 and M86935 (E. coli); EMBL Accession No. S56361 (S. typhimurium)), a glucosyltransferase (Swiss-Prot Accession No. P25740 (E. coli), an a 1,2-glucosyltransferase (rfaJ) (Swiss-Prot Accession No. P27129 (E. coli) and Swiss-Prot Accession No. P19817 (S. typhimurium)), and an β1,2-N-acetylglucosaminyltransferase (rfaK) (EMBL Accession No. U00039 (E. coli). Other glycosyltransferases for which amino acid sequences are known include those that are encoded by operons such as rfaB, which have been characterized in organisms such as Klebsiella pneumoniae, E. coli, Salmonella typhimurium, Salmonella enterica, Yersinia enterocolitica, Mycobacterium leprosum, and the rhl operon of Pseudomonas aeruginosa.

Also suitable for use in the present invention are glycosyltransferases that are involved in producing structures containing lacto-N-neotetraose, D-galactosyl-β-1,4-N-acetyl-D-glucosaminyl-β-1,3-D-galactosyl-β-1,4-D-glucose, and the P^{k} blood group trisaccharide sequence, D-galactosyl-α-1,4-D-galactosyl-β-1,4-D-glucose, which have been identified in the LOS of the mucosal pathogens Neisseria gonnorhoeae and N. meningitidis (Scholten et al., J. Med. Microbiol. 41: 236-243 (1994)). The genes from N. meningitidis and N. gonorrhoeae that encode the glycosyltransferases involved in the biosynthesis of these structures have been identified from N. meningitidis immunotypes L3 and L1 (Jennings et al., Mol. Microbiol. 18: 729-740 (1995)) and the N. gonorrhoeae mutant F62 (Gotshlich, J. Exp. Med. 180: 2181-2190 (1994)). In N. meningitidis, a locus consisting of three genes, lgtA, lgtB and lg E, encodes the glycosyltransferase enzymes required for addition of the last three of the sugars in the lacto-N-neotetraose chain (Wakarchuk et al., J. Biol. Chem. 271: 19166-73 (1996)). Recently the enzymatic activity of the lgtB and lgtA gene product was demonstrated, providing the first direct evidence for their proposed glycosyltransferase function (Wakarchuk et al., J. Biol. Chem. 271(45): 28271-276 (1996)). In N. gonorrhoeae, there are two additional genes, lgtD which adds β-D-GalNAc to the 3 position of the terminal galactose of the lacto-N-neotetraose structure and lgtC which adds a terminal α-D-Gal to the lactose element of a truncated LOS, thus creating the P^{k} blood group antigen structure (Gotshlich (1994), supra.). In N. meningitidis, a separate immunotype LI also expresses the P^{k} blood group antigen and has been shown to carry an lgtC gene (Jennings et al., (1995), supra.). Neisseria glycosyltransferases and associated genes are also described in U.S. Pat. No. 5,545,553 (Gotschlich). Genes for α1,2-fucosyltransferase and α1,3-fucosyltransferase from Helicobacter pylori has also been characterized (Martin et al., J. Biol. Chem. 272: 21349-21356 (1997)). Also of use in the present invention are the glycosyltransferases of Campylobacter jejuni (see, for example, http://afmb.cnrs-mrs.fr/˜pedro/CAZY/gtf_{—}42.html).

### a) Fucosyltransferases

In some embodiments, a glycosyltransferase used in the method of the invention is a fucosyltransferase. Fucosyltransferases are known to those of skill in the art. Exemplary fucosyltransferases include enzymes, which transfer L-fucose from GDP-fucose to a hydroxy position of an acceptor sugar. Fucosyltransferases that transfer non-nucleotide sugars to an acceptor are also of use in the present invention.

In some embodiments, the acceptor sugar is, for example, the GlcNAc in a Galβ(1→3,4)GlcNAcβ-group in an oligosaccharide glycoside. Suitable fucosyltransferases for this reaction include the Galβ(1→3,4)GlcNAcβ1-α(1→3,4)fucosyltransferase (FTIII E.C. No. 2.4.1.65), which was first characterized from human milk (see, Palcic, et al., Carbohydrate Res. 190:1-11 (1989); Prieels, et al., J. Biol. Chem. 256: 10456-10463 (1981); and Nunez, et al., Can. J. Chem. 59: 2086-2095 (1981)) and the Galβ(1→4)GlcNAcβ-αfucosyltransferases (FTIV, FTV, FTVI) which are found in human serum. FTVII (E.C. No. 2.4.1.65), a sialyl α(2→3)Galβ((1→3)GlcNAcβ fucosyltransferase, has also been characterized. A recombinant form of the Galβ(1→3,4) GlcNAcβ-α(1→3,4)fucosyltransferase has also been characterized (see, Dumas, et al., Bioorg. Med. Letters 1: 425-428 (1991) and Kukowska-Latallo, et al., Genes and Development 4: 1288-1303 (1990)). Other exemplary fucosyltransferases include, for example, α1,2 fucosyltransferase (E.C. No. 2.4.1.69). Enzymatic fucosylation can be carried out by the methods described in Mollicone, et al., Eur. J. Biochem. 191: 169-176 (1990) or U.S. Pat. No. 5,374,655. Cells that are used to produce a fucosyltransferase will also include an enzymatic system for synthesizing GDP-fucose.

### b) Galactosyltransferases

In another group of embodiments, the glycosyltransferase is a galactosyltransferase. Exemplary galactosyltransferases include α(1,3) galactosyltransferases (E.C. No. 2.4.1.151, see, e.g., Dabkowski et al., Transplant Proc. 25:2921 (1993) and Yamamoto et al. Nature 345: 229-233 (1990), bovine (GenBank j04989, Joziasse et al., J. Biol. Chem. 264: 14290-14297 (1989)), murine (GenBank m26925; Larsen et al., Proc. Nat'l. Acad. Sci. USA 86: 8227-8231 (1989)), porcine (GenBank L36152; Strahan et al., Immunogenetics 41: 101-105 (1995)). Another suitable α1,3 galactosyltransferase is that which is involved in synthesis of the blood group B antigen (EC 2.4.1.37, Yamamoto et al., J. Biol. Chem. 265: 1146-1151 (1990) (human)). Yet a further exemplary galactosyltransferase is core Gal-T1.

Also suitable for use in the methods of the invention are β(1,4) galactosyltransferases, which include, for example, EC 2.4.1.90 (LacNAc synthetase) and EC 2.4.1.22 (lactose synthetase) (bovine (D'Agostaro et al., Eur. J. Biochem. 183: 211-217 (1989)), human (Masri et al., Biochem. Biophys. Res. Commun. 157: 657-663 (1988)), murine (Nakazawa et al., J. Biochem. 104: 165-168 (1988)), as well as E.C. 2.4.1.38 and the ceramide galactosyltransferase (EC 2.4.1.45, Stahl et al., J. Neurosci. Res. 38: 234-242 (1994)). Other suitable galactosyltransferases include, for example, α1,2 galactosyltransferases (from e.g., Schizosaccharomyces pombe, Chapell et al., Mol. Biol. Cell 5: 519-528 (1994)).

Also suitable in the practice of the invention are r soluble forms of α1,3-galactosyltransferase such as that reported by Cho, S. K. and Cummings, R. D. (1997) J. Biol. Chem., 272, 13622-13628.

### c) Sialyltransferases

Sialyltransferases are another type of glycosyltransferase that is useful in the recombinant cells and reaction mixtures of the invention. Cells that produce recombinant sialyltransferases will also produce CMP-sialic acid, which is a sialic acid donor for sialyltransferases. Examples of sialyltransferases that are suitable for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al., Glycobiology 6: v-xiv (1996)). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem. 256: 3159 (1981), Weinstein et al., J. Biol. Chem. 257: 13845 (1982) and Wen et al., J. Biol. Chem. 267: 21011 (1992). Another exemplary α-2,3-sialyltransferase (EC 2.4.99.4) transfers sialic acid to the non-reducing terminal Gal of the disaccharide or glycoside. see, Rearick et al., J. Biol. Chem. 254: 4444 (1979) and Gillespie et al., J. Biol. Chem. 267: 21004 (1992). Further exemplary enzymes include Gal-β-1,4-GlcNAc α-2,6 sialyltransferase (See, Kurosawa et al. Eur. J. Biochem. 219: 375-381 (1994)).

Preferably, for glycosylation of carbohydrates of glycopeptides the sialyltransferase will be able to transfer sialic acid to the sequence Galβ1,4GlcNAc-, the most common penultimate sequence underlying the terminal sialic acid on fully sialylated carbohydrate structures (see, Table 5).

As an example, when a modified sialic acid is used, a sialyltransferase or a trans-sialidase (for α2,3-linked sialic acid only) can be used in these methods.

TABLE 5
Sialyltransferases which use the Galβ1,4GlcNAc sequence as an acceptor
substrate

| Sialyltransferase | Source | Sequence(s) formed | Ref. |
|---|---|---|---|
| ST6Gal I | Mammalian | NeuAcα2,6Galβl,4GlcNAc- | 1 |
| ST3Gal III | Mammalian | NeuAcα2,3Galβ1,4GlcNAc- | 1 |
| | | NeuAcα2,3Galβ1,3GlcNAc- | |
| ST3Gal IV | Mammalian | NeuAcα2,3Galβ1,4GlcNAc- | 1 |
| | | NeuAcα2,3Galβ1,3GlcNAc- | |
| ST6Gal II | Mammalian | NeuAcα2,6Galβ1,4GlcNAc | |
| ST6Gal II | photobacterium | NeuAcα2,6Galβ1,4GlcNAc- | 2 |
| ST3Gal V | N. meningitides | NeuAcα2,3Galβ1,4GlcNAc- | 3 |
| | N. gonorrhoeae | | |

1) Goochee et al., Bio/Technology 9: 1347-1355 (1991)
2) Yamamoto et al., J. Biochem. 120: 104-110 (1996)
3) Gilbert et al., J. Biol. Chem. 271: 28271-28276 (1996)

An example of a sialyltransferase that is useful in the claimed methods is ST3Gal III, which is also referred to as α(2,3)sialyltransferase (EC 2.4.99.6). This enzyme catalyzes the transfer of sialic acid to the Gal of a Galβ1,3GlcNAc or Galβ1,4GlcNAc glycoside (see, e.g., Wen et al., J. Biol. Chem. 267: 21011 (1992); Van den Eijnden et al., J. Biol. Chem. 256: 3159 (1991)) and is responsible for sialylation of asparagine-linked oligosaccharides in glycopeptides. The sialic acid is linked to a Gal with the formation of an α-linkage between the two saccharides. Bonding (linkage) between the saccharides is between the 2-position of NeuAc and the 3-position of Gal. This particular enzyme can be isolated from rat liver (Weinstein et al., J. Biol. Chem. 257: 13845 (1982)); the human cDNA (Sasaki et al. (1993) J. Biol. Chem. 268: 22782-22787; Kitagawa & Paulson (1994) J. Biol. Chem. 269: 1394-1401) and genomic (Kitagawa et al. (1996) J. Biol. Chem. 271: 931-938) DNA sequences are known, facilitating production of this enzyme by recombinant expression. In another embodiment, the claimed sialylation methods use a rat ST3Gal III.

Other exemplary sialyltransferases of use in the present invention include those isolated from Campylobacter jejuni, including the α(2,3). See, e.g, WO99/49051.

Sialyltransferases other those listed in Table 5, are also useful in an economic and efficient large-scale process for sialylation of commercially important glycopeptides. As a simple test to find out the utility of these other enzymes, various amounts of each enzyme (1-100 mU/mg protein) are reacted with asialo-α₁ AGP (at 1-10 mg/ml) to compare the ability of the sialyltransferase of interest to sialylate glycopeptides relative to either bovine ST6Gal I, ST3Gal III or both sialyltransferases. Alternatively, other glycopeptides or glycopeptides, or N-linked oligosaccharides enzymatically released from the peptide backbone can be used in place of asialo-α₁ AGP for this evaluation. Sialyltransferases with the ability to sialylate N-linked oligosaccharides of glycopeptides more efficiently than ST6Gal I are useful in a practical large-scale process for peptide sialylation (as illustrated for ST3Gal III in this disclosure). Other exemplary sialyltransferases are shown in FIG. 10.

### d) GalNAc Transferases

N-acetylgalactosaminyltransferases are of use in practicing the present invention, particularly for binding a GalNAc moiety to an amino acid of the O-linked glycosylation site of the peptide. Suitable N-acetylgalactosaminyltransferases include, but are not limited to, α(1,3) N-acetylgalactosaminyltransferase, β(1,4) N-acetylgalactosaminyltransferases (Nagata et al., J. Biol. Chem. 267: 12082-12089 (1992) and Smith et al., J. Biol. Chem. 269: 15162 (1994)) and polypeptide N-acetylgalactosaminyltransferase (Homa et al., J. Biol. Chem. 268: 12609 (1993)).

Production of proteins such as the enzyme GalNAc T_{I-XX} from cloned genes by genetic engineering is well known. See, eg., U.S. Pat. No. 4,761,371. One method involves collection of sufficient samples, then the amino acid sequence of the enzyme is determined by N-terminal sequencing. This information is then used to isolate a cDNA clone encoding a full-length (membrane bound) transferase which upon expression in the insect cell line Sf9 resulted in the synthesis of a fully active enzyme. The acceptor specificity of the enzyme is then determined using a semiquantitative analysis of the amino acids surrounding known glycosylation sites in 16 different proteins followed by in vitro glycosylation studies of synthetic peptides. This work has demonstrated that certain amino acid residues are overrepresented in glycosylated peptide segments and that residues in specific positions surrounding glycosylated serine and threonine residues may have a more marked influence on acceptor efficiency than other amino acid moieties.

### Sulfotransferases

The invention also provides methods for producing peptides that include sulfated molecules, including, for example sulfated polysaccharides such as heparin, heparan sulfate, carragenen, and related compounds. Suitable sulfotransferases include, for example, chondroitin-6-sulphotransferase (chicken cDNA described by Fukuta et al., J. Biol. Chem. 270: 18575-18580 (1995); GenBank Accession No. D49915), glycosaminoglycan N-acetylglucosamine N-deacetylase/N-sulphotransferase 1 (Dixon et al., Genomics 26: 239-241 (1995); UL18918), and glycosaminoglycan N-acetylglucosamine N-deacetylase/N-sulphotransferase 2 (murine cDNA described in Orellana et al., J. Biol. Chem. 269: 2270-2276 (1994) and Eriksson et al., J. Biol. Chem. 269: 10438-10443 (1994); human cDNA described in GenBank Accession No. U2304).

### Cell-Bound Glycosyltransferases

In another embodiment, the enzymes utilized in the method of the invention are cell-bound glycosyltransferases. Although many soluble glycosyltransferases are known (see, for example, U.S. Pat. No. 5,032,519), glycosyltransferases are generally in membrane-bound form when associated with cells. Many of the membrane-bound enzymes studied thus far are considered to be intrinsic proteins; that is, they are not released from the membranes by sonication and require detergents for solubilization. Surface glycosyltransferases have been identified on the surfaces of vertebrate and invertebrate cells, and it has also been recognized that these surface transferases maintain catalytic activity under physiological conditions. However, the more recognized function of cell surface glycosyltransferases is for intercellular recognition (Roth, MOLECULAR APPROACHES to SUPRACELLULAR PHENOMENA, 1990).

Methods have been developed to alter the glycosyltransferases expressed by cells. For example, Larsen et al., Proc. Natl. Acad. Sci. USA 86: 8227-8231 (1989), report a genetic approach to isolate cloned cDNA sequences that determine expression of cell surface oligosaccharide structures and their cognate glycosyltransferases. A cDNA library generated from mRNA isolated from a murine cell line known to express UDP-galactose: .β.-D-galactosyl-1,4-N-acetyl-D-glucosaminide α-1,3-galactosyltransferase was transfected into COS-1 cells. The transfected cells were then cultured and assayed for a 1-3 galactosyltransferase activity.

Francisco et al., Proc. Natl. Acad. Sci. USA 89: 2713-2717 (1992), disclose a method of anchoring β-lactamase to the external surface of Escherichia coli. A tripartite fusion consisting of (i) a signal sequence of an outer membrane protein, (ii) a membrane-spanning section of an outer membrane protein, and (iii) a complete mature β-lactamase sequence is produced resulting in an active surface bound β-lactamase molecule. However, the Francisco method is limited only to procaryotic cell systems and as recognized by the authors, requires the complete tripartite fusion for proper functioning.

### Fusion Proteins

In other exemplary embodiments, the methods of the invention utilize fusion proteins that have more than one enzymatic activity that is involved in synthesis of a desired glycopeptide conjugate. The fusion polypeptides can be composed of, for example, a catalytically active domain of a glycosyltransferase that is joined to a catalytically active domain of an accessory enzyme. The accessory enzyme catalytic domain can, for example, catalyze a step in the formation of a nucleotide sugar that is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressible nucleotide sequence. In other embodiments the fusion protein includes the catalytically active domains of two or more glycosyltransferases. See, for example, U.S. Pat. No. 5,641,668. The modified glycopeptides of the present invention can be readily designed and manufactured utilizing various suitable fusion proteins (see, for example, PCT Patent Application PCT/CA98/01180, which was published as WO 99/31224 on Jun. 24, 1999.)

### Immobilized Enzymes

In addition to cell-bound enzymes, the present invention also provides for the use of enzymes that are immobilized on a solid and/or soluble support. In an exemplary embodiment, there is provided a glycosyltransferase that is conjugated to a PEG via an intact glycosyl linker according to the methods of the invention. The PEG-linker-enzyme conjugate is optionally attached to solid support. The use of solid supported enzymes in the methods of the invention simplifies the work up of the reaction mixture and purification of the reaction product, and also enables the facile recovery of the enzyme. The glycosyltransferase conjugate is utilized in the methods of the invention. Other combinations of enzymes and supports will be apparent to those of skill in the art.

### Purification of Peptide Conjugates

The products produced by the above processes can be used without purification. However, it is usually preferred to recover the product. Standard, well-known techniques for recovery of glycosylated saccharides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have molecular weight cutoff of about 3000 to about 10,000 can be used to remove proteins such as glycosyl transferases. Nanofiltration or reverse osmosis can then be used to remove salts and/or purify the product saccharides (see, e.g., WO 98/15581). Nanofilter membranes are a class of reverse osmosis membranes that pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

If the modified glycoprotein is produced intracellularly, as a first step, the particulate debris, either host cells or lysed fragments, is removed, for example, by centrifugation or ultrafiltration; optionally, the protein may be concentrated with a commercially available protein concentration filter, followed by separating the polypeptide variant from other impurities by one or more steps selected from immunoaffinity chromatography, ion-exchange column fractionation (e.g., on diethylaminoethyl (DEAE) or matrices containing carboxymethyl or sulfopropyl groups), chromatography on Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, SP-Sepharose, or protein A Sepharose, SDS-PAGE chromatography, silica chromatography, chromatofocusing, reverse phase HPLC (e.g., silica gel with appended aliphatic groups), gel filtration using, e.g., Sephadex molecular sieve or size-exclusion chromatography, chromatography on columns that selectively bind the polypeptide, and ethanol or ammonium sulfate precipitation.

Modified glycopeptides produced in culture are usually isolated by initial extraction from cells, enzymes, etc., followed by one or more concentration, salting-out, aqueous ion-exchange, or size-exclusion chromatography steps, e.g., SP Sepharose. Additionally, the modified glycoprotein may be purified by affinity chromatography. HPLC may also be employed for one or more purification steps.

A protease inhibitor, e.g., methylsulfonylfluoride (PMSF) may be included in any of the foregoing steps to inhibit proteolysis and antibiotics may be included to prevent the growth of adventitious contaminants.

Within another embodiment, supernatants from systems which sproduce the modified glycopeptide of the invention are first concentrated using a commercially available protein concentration filter, for example, an Amicon or Millipore Pellicon ultrafiltration unit. Following the concentration step, the concentrate may be applied to a suitable purification matrix. For example, a suitable affinity matrix may comprise a ligand for the peptide, a lectin or antibody molecule bound to a suitable support. Alternatively, an anion-exchange resin may be employed, for example, a matrix or substrate having pendant DEAE groups. Suitable matrices include acrylamide, agarose, dextran, cellulose, or other types commonly employed in protein purification. Alternatively, a cation-exchange step may be employed. Suitable cation exchangers include various insoluble matrices comprising sulfopropyl or carboxymethyl groups. Sulfopropyl groups are particularly preferred.

Finally, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

The modified glycopeptide of the invention resulting from a large-scale fermentation may be purified by methods analogous to those disclosed by Urdal et al., J. Chromatog. 296: 171 (1984). This reference describes two sequential, RP-HPLC steps for purification of recombinant human IL-2 on a preparative HPLC column. Alternatively, techniques such as affinity chromatography may be utilized to purify the modified glycoprotein.

### Methods of Treatment

In addition to the conjugates discussed above, the present invention provides methods of preventing, curing or ameliorating a disease state by administering a conjugate of the invention to a subject at risk of developing the disease or a subject that has the disease. Additionally, the invention provides methods for targeting conjugates of the invention to a particular tissue or region of the body.

### EXEMPLARY EMBODIMENTS

In one embodiment, the application provides a polypeptide conjugate comprising a structure according to Formula (I):

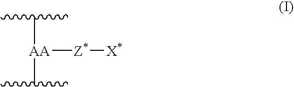

wherein
AA is an aromatic amino acid residue of said polypeptide;
Z* is a member selected from a bond, a glycosyl mimetic moiety and a glycosyl moiety, which is a member selected from a monosaccharide and an oligosaccharide; and
X* is a member selected from a modifying group, a glycosyl linking group, and a glycosyl linking group that comprises a modifying group.

The polypeptide according to any of the above embodiments, wherein said polypeptide is a member selected from bone morphogenetic protein 2 (BMP-2), bone morphogenetic protein 7 (BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), human chorionic gonadotropin (hCG), alpha-galactosidase, alpha-iduronidase, beta-glucosidase, alpha-galactosidase A, anti-thrombin III (AT III), follicle stimulating hormone, glucagon-like peptide-1 (GLP-1), glucagon-like peptide-2 (GLP-2), fibroblast growth factor 7 (FGF-7), fibroblast growth factor 21 (FGF-21), fibroblast growth factor 23 (FGF-23), Factor VII, Factor VIII, B-domain deleted Factor VIII, Factor IX, prokinetisin, extendin-4, anti-TNF-alpha monoclonal antibody, TNF receptor-IgG Fc region fusion protein, anti-HER2 monoclonal antibody, monoclonal antibody to protein F of respiratory syncytial virus, monoclonal antibody to TNF-α, monoclonal antibody to glycoprotein IIb/IIIa, monoclonal antibody to CD20, monoclonal antibody to VEGF-A, and mutants thereof.

The polypeptide conjugate according to any of the above embodiments, wherein said aromatic amino acid is tryptophan (W).

The polypeptide conjugate according to any of the above embodiments, wherein said tryptophan is glycosylated at the C²- or N¹-position of the indole moiety.

The polypeptide conjugate according to any of the above embodiments, wherein Z* comprises a mannosyl (Man) moiety.

The polypeptide conjugate according to any of the above embodiments,

wherein Z* is a bond and X* is a mannosyl (Man) moiety comprising a modifying group.

The polypeptide conjugate according to any of the above embodiments, wherein said aromatic amino acid is part of a glycosylation consensus sequence.

The polypeptide conjugate according to any of the above embodiments, wherein said glycosylation consensus sequence comprises an amino acid sequence, which is a member selected from: WX¹(X²W)ₘ; WX¹X²WX³(X⁴W)ₙ; WX¹X²C; WX¹X²WX³X⁴C; WX¹X²WX³X⁴WX⁵X⁶C (SEQ ID NO: 1); and WX¹X²WX³X⁴X⁵X⁶X⁷C

wherein
m and n are integers from 0-1;
W is tryptophan;
C is cysteine; and
X¹, X², X³, X⁴, X⁵, X⁶ and X⁷ are members independently selected from glutamic acid (E), glutamine (Q), aspartic acid (D), asparagine (N), threonine (T), serine (S) and uncharged amino acids.

The polypeptide conjugate according to any of the above embodiments, wherein X¹, X³ and X⁵ are members independently selected from serine (S), threonine (T) and uncharged amino acids.

The polypeptide conjugate according to any of the above embodiments, wherein said modifying group is a non-glycosidic modifying group.

The polypeptide conjugate according to any of the above embodiments, wherein said non-glycosidic modifying group is a member selected from linear and branched and comprises one or more polymeric moiety, wherein each polymeric moiety is independently selected.

The polypeptide conjugate according to any of the above embodiments, wherein said polymeric moiety is a member selected from poly(ethylene glycol) and derivatives thereof.

The polypeptide conjugate according to any of the above embodiments, wherein Z* comprises a member selected from a mannosyl (Man) moiety, a galactosyl (Gal) moiety, a GalNAc moiety, a GlcNAc moiety, a xylosyl (Xyl) moiety, a glucosyl (Glc) moiety, a sialyl (Sia) moiety and combinations thereof.

The polypeptide conjugate according to any of the above embodiments, wherein X* comprises a moiety, which is a member selected from a mannosyl (Man) moiety, a sialyl (Sia) moiety, a galactosyl (Gal) moiety, a GlcNAc moiety, a GalNAc moiety and a Gal-Sia moiety.

The polypeptide conjugate according to any of the above embodiments, wherein X* comprises a moiety according to Formula (II):

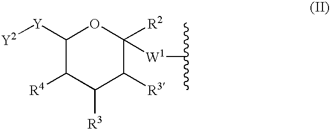

wherein
W¹ is a member selected from a bond, S and O;
R² is a member selected from H, —R¹, —CH₂R¹, and —C(X¹)R¹
wherein
   R¹ is a member selected from OR⁹, SR⁹, NR¹⁰R¹¹, substituted or unsubstituted alkyl and substituted or unsubstituted heteroalkyl
   wherein
      R⁹ is a member selected from H, a metal ion, substituted or unsubstituted alkyl, substituted or unsubstituted heteroalkyl and acyl;
      R¹⁰ and R¹¹ are members independently selected from H, substituted or unsubstituted alkyl, substituted or unsubstituted heteroalkyl and acyl;
   X¹ is a member selected from substituted or unsubstituted alkyl, O, S and NR⁸ wherein
      R⁸ is a member selected from H, OH, substituted or unsubstituted alkyl and substituted or unsubstituted heteroalkyl;
Y is a member selected from CH₂, CH(OH)CH₂, CH(OH)CH(OH)CH₂, CH, CH(OH)CH; CH(OH)CH(OH)CH, CH(OH), CH(OH)CH(OH), and CH(OH)CH(OH)CH(OH);
Y² is a member selected from H, OR⁶, R⁶, substituted or unsubstituted alkyl, substituted or unsubstituted heteroalkyl,

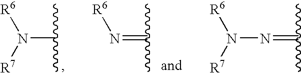

wherein
   R⁶ and R⁷ are members independently selected from H, L^{a}-R^{6b}, C(O)R^{6b}, C(O)-L^{a}-R^{6b}, substituted or unsubstituted alkyl and substituted or unsubstituted heteroalkyl
   wherein
      L^{a} is a member selected from a bond and a linker group; and
      R^{6b} is a member selected from H, substituted or unsubstituted alkyl, substituted or unsubstituted heteroalkyl and a modifying group;
   R³, R^{3′} and R⁴ are members independently selected from H, OR^{3″}, substituted or unsubstituted alkyl, substituted or unsubstituted heteroalkyl, L^{aa}-R^{6c}, C(O)R^{6c}, C(O)-L^{aa}-R^{6c}, NHC(O)R^{6c}
   wherein
      each R^{3″} is a member independently selected from H, substituted or unsubstituted alkyl and substituted or unsubstituted heteroalkyl;
      L^{aa} is a member selected from a bond and a linker group; and
      R^{6c} is a member selected from H, substituted or unsubstituted alkyl, substituted or unsubstituted heteroalkyl, substituted or unsubstituted aryl, substituted or unsubstituted heteroaryl, substituted or unsubstituted heterocycloalkyl, NR¹³R¹⁴ and a modifying group
   wherein
      R¹³ and R¹⁴ are members independently selected from H, substituted or unsubstituted alkyl and substituted or unsubstituted heteroalkyl.

The polypeptide conjugate according to any of the above embodiments, wherein X* comprises a moiety according to Formula (III):

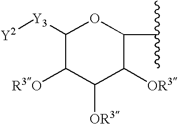

wherein Y³ is a member selected from CH and CH₂.

The polypeptide conjugate according to any of the above embodiments, wherein X* comprises a moiety according to Formula (IV):

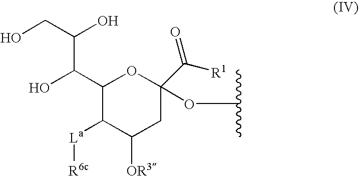

The polypeptide conjugate according to any of the above embodiments, wherein at least one of R^{6b} and R^{6c} is a member selected from:

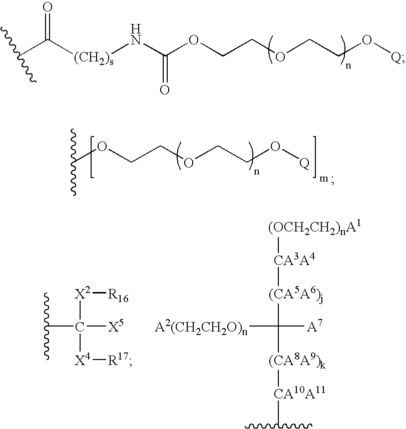

wherein
s, j and k are integers independently selected from 0 to 20;
each n is an integer independently selected from 0 to 2500;
m is an integer from 1-5;
Q is a member selected from H and C₁-C₆ alkyl;
R¹⁶ and R¹⁷ are independently selected polymeric moieties;
X² and X⁴ are independently selected linkage fragments joining polymeric moieties R¹⁶ and R¹⁷ to C; and
X⁵ is a non-reactive group;
A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NA¹²A¹³, —OA¹² and —SiA¹²A¹³
   wherein
      A¹² and A¹³ are members independently selected from substituted or unsubstituted alkyl, substituted or unsubstituted heteroalkyl, substituted or unsubstituted heterocycloalkyl, substituted or unsubstituted aryl, and substituted or unsubstituted heteroaryl.

The invention further provides a method for making a polypeptide conjugate according to any of the above embodiments, comprising the steps of:

(i) recombinantly producing said polypeptide, and

(ii) enzymatically glycosylating said polypeptide at said aromatic amino acid residue.

The invention further provides a polypeptide conjugate derived from a non-naturally occurring polypeptide, said polypeptide conjugate comprising a structure according to Formula (V):

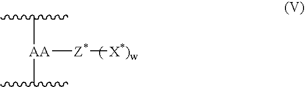

wherein
w is an integer selected from 0 to 1;
AA is an aromatic amino acid residue of said non-naturally occurring polypeptide;
Z* is a member selected from a bond, a glycosyl mimetic moiety and a glycosyl moiety, which is a member selected from mono- and oligosaccharides; and
X* is a member selected from a modifying group, a glycosyl linking group and a glycosyl linking group comprising a modifying group.

The polypeptide conjugate according to any of the above embodiments, wherein said non-naturally occurring polypeptide has an amino acid sequence comprising a glycosylation consensus sequence, said glycosylation consensus sequence comprising said aromatic amino acid.

The polypeptide conjugate according to any of the above embodiments, wherein said non-naturally occurring polypeptide is derived from a parent polypeptide, which is a member selected from bone morphogenetic protein 2 (BMP-2), bone morphogenetic protein 7 (BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), human chorionic gonadotropin (hCG), alpha-galactosidase, alpha-iduronidase, beta-glucosidase, alpha-galactosidase A, anti-thrombin III (AT III), follicle stimulating hormone, glucagon-like peptide-1 (GLP-1), glucagon-like peptide-2 (GLP-2), fibroblast growth factor 7 (FGF-7), fibroblast growth factor 21 (FGF-21), fibroblast growth factor 23 (FGF-23), Factor VII, Factor VIII, B-domain deleted Factor VIII, Factor IX, prokinetisin, extendin-4, anti-TNF-alpha monoclonal antibody, TNF receptor-IgG Fc region fusion protein, anti-HER2 monoclonal antibody, monoclonal antibody to protein F of respiratory syncytial virus, monoclonal antibody to TNF-α, monoclonal antibody to glycoprotein IIb/IIIa, monoclonal antibody to CD20, monoclonal antibody to VEGF-A, and mutants thereof.

The polypeptide conjugate according to any of the above embodiments, wherein said aromatic amino acid is tryptophan (W).

The polypeptide conjugate according to any of the above embodiments, wherein said non-naturally occurring polypeptide is glycosylated at the C²- or N¹-position of the indole moiety of said tryptophan.

The invention further provides an isolated nucleic acid encoding said non-naturally occurring polypeptide according to any of the above embodiments.

The invention further provides an expression cassette comprising said nucleic acid according to any of the above embodiments.

The invention further provides a cell comprising said nucleic acid according to any of the above embodiments.

The invention further provides a polypeptide conjugate comprising:
a) a polypeptide; and
b) a modifying group, wherein said modifying group is covalently attached to said polypeptide at a glycosyl or amino acid residue of said polypeptide via a glycosyl mimetic linking group, wherein said glycosyl mimetic linking group comprises a structure according to Formula (VII):

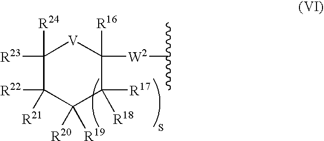

wherein
s is an integer from 0 to 3;
V and W² are members independently selected from a bond, O, S, NR¹² and CR¹³R¹⁴, wherein R¹², R¹³ and R¹⁴ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl,
   with the proviso that at least one of V and W² is other than O;
R¹⁶, R¹⁷, R¹⁸, R¹⁹, R²⁰, R²¹, R²², R²³ and R²⁴ are members independently selected from H, halogen, CN, OR⁹, SR⁹, NR¹⁰R¹¹, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
   at least two of R¹⁶, R¹⁷, R¹⁸, R¹⁹, R²⁰, R²¹, R²², R²³ and R²⁴, together with the atoms to which they are attached, are optionally joined to form a 5- to 7-membered ring;
   R⁹ is a member independently selected from H, a metal ion, substituted or unsubstituted alkyl, substituted or unsubstituted heteroalkyl and acyl;
   R¹⁰ and R¹¹ are members independently selected from H, substituted or unsubstituted alkyl, substituted or unsubstituted heteroalkyl and acyl.

The polypeptide conjugate according to any of the above embodiments wherein said polypeptide is a member selected from bone morphogenetic protein 2 (BMP-2), bone morphogenetic protein 7 (BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), human chorionic gonadotropin (hCG), alpha-galactosidase, alpha-iduronidase, beta-glucosidase, alpha-galactosidase A, anti-thrombin III (AT III), follicle stimulating hormone, glucagon-like peptide-1 (GLP-1), glucagon-like peptide-2 (GLP-2), fibroblast growth factor 7 (FGF-7), fibroblast growth factor 21 (FGF-21), fibroblast growth factor 23 (FGF-23), Factor VII, Factor VIII, B-domain deleted Factor VIII, Factor IX, prokinetisin, extendin-4, anti-TNF-alpha monoclonal antibody, TNF receptor-IgG Fc region fusion protein, anti-HER2 monoclonal antibody, monoclonal antibody to protein F of respiratory syncytial virus, monoclonal antibody to TNF-α, monoclonal antibody to glycoprotein IIb/IIIa, monoclonal antibody to CD20, monoclonal antibody to VEGF-A, and variants and mutants thereof.

The invention further provides a pharmaceutical composition comprising a polypeptide conjugate according to any of the above embodiments and a pharmaceutically acceptable carrier, vehicle or diluent.

The invention further provides a method of making a polypeptide conjugate according to any of the above embodiments said method comprising:
(i) contacting a polypeptide or glycopeptide and a linking moiety in the presence of an enzyme, under conditions sufficient for said enzyme to form a covalent bond between said polypeptide or said glycopeptide and said linking moiety, wherein a member selected from said polypeptide or said glycopeptide and
   said linking moiety comprises a nucleophile and wherein the other member comprises a leaving group,
      thereby creating said polypeptide conjugate.

The method according to any of the above embodiments wherein said linking moiety is a member selected from a glycosyl moiety and a glycosyl mimetic moiety.

The method according to any of the above embodiments, wherein said enzyme is a member selected from a glycosyltransferase, a glycosynthase (GS) and a glycohydrase (GH).

The method according to any of the above embodiments, wherein said glycosynthase is a non-naturally occurring glycosidase.

The method according to any of the above embodiments, wherein said leaving group is a member selected from a halogen, an azide, a tosylate, a mesylate, an —O-nitrophenyl and an —O-dinitrophenyl group.

A polypeptide conjugate according to any of the above embodiments, wherein V is O and W² is S.

The polypeptide conjugate according to any of the above embodiments wherein said glycosyl mimetic linking group comprises a structure according to Formula (VII):

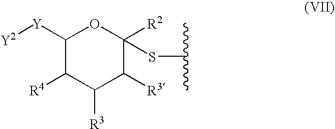

wherein
R² is a member selected from H, —R¹, —CH₂R¹, and —C(X¹)R¹
wherein
   R¹ is a member selected from OR⁹, SR⁹, NR⁹R¹⁰, substituted or unsubstituted alkyl and substituted or unsubstituted heteroalkyl
   wherein
      R⁹ and R¹⁰ are members independently selected from H, a metal ion, substituted or unsubstituted alkyl, substituted or unsubstituted heteroalkyl and acyl;
   X¹ is a member selected from substituted or unsubstituted alkyl, O, S and NR⁸ wherein
      R⁸ is a member selected from H, OH, substituted or unsubstituted alkyl and substituted or unsubstituted heteroalkyl;
Y is a member selected from CH₂, CH(OH)CH₂, CH(OH)CH(OH)CH₂, CH, CH(OH)CH; CH(OH)CH(OH)CH, CH(OH), CH(OH)CH(OH), and CH(OH)CH(OH)CH(OH);
Y² is a member selected from H, OR⁶, R⁶, substituted or unsubstituted alkyl, substituted or unsubstituted heteroalkyl,

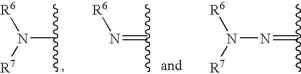

wherein
   R⁶ and R⁷ are members independently selected from H, L^{a}-R^{6b}, C(O)R^{6b}, C(O)-L^{a}-R^{6b}, substituted or unsubstituted alkyl and substituted or unsubstituted heteroalkyl
   wherein
      L^{a} is a member selected from a bond and a linker group; and
      R^{6b} is a member selected from H, substituted or unsubstituted alkyl, substituted or unsubstituted heteroalkyl and a modifying group;
R³ is a member selected from H, substituted or unsubstituted alkyl, substituted or unsubstituted heteroalkyl, and OR^{3″}, wherein R^{3″} is a member selected from H, substituted or unsubstituted alkyl and substituted or unsubstituted heteroalkyl;
R^{3′} and R⁴ are members independently selected from H, OH, substituted or unsubstituted alkyl, substituted or unsubstituted heteroalkyl, L^{aa}-R^{6c}, C(O)R^{6c}, C(O)-L^{aa}-R^{6c}, NHC(O)R^{6c}
   wherein
      L^{aa} is a member selected from a bond and a linker group; and
      R^{6c} is a member selected from H, substituted or unsubstituted alkyl, substituted or unsubstituted heteroalkyl, substituted or unsubstituted aryl, substituted or unsubstituted heteroaryl, substituted or unsubstituted heterocycloalkyl, NR¹³R¹⁴ and a modifying group
      wherein
         R¹³ and R¹⁴ are members independently selected from H, substituted or unsubstituted alkyl and substituted or unsubstituted heteroalkyl.

The invention further provides a method of making a polypeptide conjugate according to any of the above embodiments, said method comprising:
(i) contacting a glycopeptide and a glycosyl moiety in the presence of a thioglycoligase, under conditions sufficient for said thioglycoligase to form a covalent bond between said glycopeptide and said glycosyl moiety, wherein a member selected from said glycopeptide and said glycosyl moiety comprises a sulfhydryl group,
   thereby creating said polypeptide conjugate.

The method according to any of the above embodiments, wherein said thioglycoligase is a mutant glycosidase.

The method according to any of the above embodiments, wherein said glycosidase is a member selected from a glucosidase, a mannosidase, a glucuronidase, a sialydase, a xylosidase and a galactosidase.

The method according to any of the above embodiments, wherein said mutant glycosidase is mutated at the acid/base position.

The method according to any of the above embodiments, wherein said thioglycoligase is recombinantly produced.

While this invention has been disclosed with reference to specific embodiments, it is apparent that other embodiments and variations of this invention may be devised by others skilled in the art without departing from the true spirit and scope of the invention.

All patents, patent applications, and other publications cited in this application are incorporated by reference in the entirety.

What is claimed is:

1. A polypeptide conjugate comprising a structure according to Formula (I): 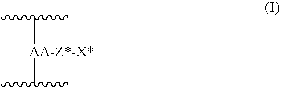
wherein
AA is an aromatic amino acid residue of said polypeptide;
Z* is a member selected from a bond, a glycosyl mimetic moiety and a glycosyl moiety, which is a member selected from a monosaccharide and an oligosaccharide; and
X* is a member selected from a modifying group, a glycosyl linking group, and a glycosyl linking group that comprises a modifying group.

2. The polypeptide conjugate of claim 1, wherein said polypeptide is a member selected from bone morphogenetic protein 2 (BMP-2), bone morphogenetic protein 7 (BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (α-1 protease inhibitor), glucocerebrosidase, tissue-type plasminogen activator (TPA), interleukin-2 (IL-2), urokinase, human DNase, insulin, hepatitis B surface protein (HbsAg), human growth hormone (hGH), human chorionic gonadotropin (hCG), alpha-galactosidase, alpha-iduronidase, beta-glucosidase, alpha-galactosidase A, anti-thrombin III (AT III), follicle stimulating hormone, glucagon-like peptide-1 (GLP-1), glucagon-like peptide-2 (GLP-2), fibroblast growth factor 7 (FGF-7), fibroblast growth factor 21 (FGF-21), fibroblast growth factor 23 (FGF-23), Factor VII, Factor VIII, B-domain deleted Factor VIII, Factor IX, prokinetisin, extendin-4, anti-TNF-alpha monoclonal antibody, TNF receptor-IgG Fc region fusion protein, anti-HER2 monoclonal antibody, monoclonal antibody to protein F of respiratory syncytial virus, monoclonal antibody to TNF-α, monoclonal antibody to glycoprotein IIb/IIIa, monoclonal antibody to CD20, monoclonal antibody to VEGF-A, and mutants thereof.

3. The polypeptide conjugate of claim 1, wherein said aromatic amino acid is tryptophan (W).

4. The polypeptide conjugate of claim 3, wherein said tryptophan is glycosylated at the C²- or N¹-position of the indole moiety.

5. The polypeptide conjugate of claim 4, wherein Z* comprises a mannosyl (Man) moiety.

6. The polypeptide conjugate of claim 4, wherein Z* is a bond and X* is a mannosyl (Man) moiety comprising a modifying group.

7. The polypeptide conjugate according to claim 1, wherein said aromatic amino acid is part of a glycosylation consensus sequence.

8. The polypeptide conjugate according to claim 7, wherein said glycosylation consensus sequence comprises an amino acid sequence, which is a member selected from: WX¹(X²W)ₘ; WX¹X²WX³(X⁴W)ₙ; WX¹X²C; WX¹X²WX³X⁴C; WX¹X²WX³X⁴WX⁵X⁶C (SEQ ID NO: 1); and WX¹X²WX³X⁴X⁵X⁶X⁷C
wherein
m and n are integers from 0-1;
W is tryptophan;
C is cysteine; and
X¹, X², X³, X⁴, X⁵, X⁶ and X⁷ are members independently selected from glutamic acid (E), glutamine (Q), aspartic acid (D), asparagine (N), threonine (T), serine (S) and uncharged amino acids.

9. The polypeptide conjugate according to claim 8, wherein X¹, X³ and X⁵ are members independently selected from serine (S), threonine (T) and uncharged amino acids.

10. The polypeptide conjugate of claim 1, wherein said modifying group is a non-glycosidic modifying group.

11. The polypeptide conjugate of claim 10, wherein said non-glycosidic modifying group is a member selected from linear and branched and comprises one or more polymeric moiety, wherein each polymeric moiety is independently selected.

12. The polypeptide conjugate of claim 11, wherein said polymeric moiety is a member selected from poly(ethylene glycol) and derivatives thereof.

13. The polypeptide conjugate of claim 1, wherein Z* comprises a member selected from a mannosyl (Man) moiety, a galactosyl (Gal) moiety, a GalNAc moiety, a GlcNAc moiety, a xylosyl (Xyl) moiety, a glucosyl (Glc) moiety, a sialyl (Sia) moiety and combinations thereof.

14. The polypeptide conjugate of claim 1, wherein X* comprises a moiety, which is a member selected from a mannosyl (Man) moiety, a sialyl (Sia) moiety, a galactosyl (Gal) moiety, a GlcNAc moiety, a GalNAc moiety and a Gal-Sia moiety.

15. The polypeptide conjugate of claim 1, wherein X* comprises a moiety according to Formula (II): 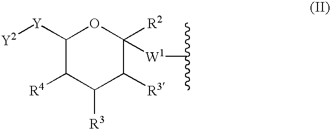
wherein
W¹ is a member selected from a bond, S and O;
R² is a member selected from H, —R¹, —CH₂R¹, and —C(X¹)R¹
wherein
R¹ is a member selected from OR⁹, SR⁹, NR¹⁰R¹¹, substituted or unsubstituted alkyl and substituted or unsubstituted heteroalkyl
wherein
R⁹ is a member selected from H, a metal ion, substituted or unsubstituted alkyl, substituted or unsubstituted heteroalkyl and acyl;
R¹⁰ and R¹¹ are members independently selected from H, substituted or unsubstituted alkyl, substituted or unsubstituted heteroalkyl and acyl;
X¹ is a member selected from substituted or unsubstituted alkyl, O, S and NR⁸
wherein
R⁸ is a member selected from H, OH, substituted or unsubstituted alkyl and substituted or unsubstituted heteroalkyl;
Y is a member selected from CH₂, CH(OH)CH₂, CH(OH)CH(OH)CH₂, CH, CH(OH)CH; CH(OH)CH(OH)CH, CH(OH), CH(OH)CH(OH), and CH(OH)CH(OH)CH(OH);
Y² is a member selected from H, OR⁶, R⁶, substituted or unsubstituted alkyl, substituted or unsubstituted heteroalkyl, 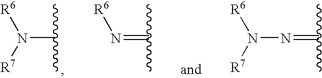
wherein
R⁶ and R⁷ are members independently selected from H, L^{a}-R^{6b}, C(O)R^{6b}, C(O)-L^{a}-R^{6b}, substituted or unsubstituted alkyl and substituted or unsubstituted heteroalkyl
wherein
L^{a} is a member selected from a bond and a linker group; and
R^{6b} is a member selected from H, substituted or unsubstituted alkyl, substituted or unsubstituted heteroalkyl and a modifying group;
R³, R^{3′} and R⁴ are members independently selected from H, OR^{3″}, substituted or unsubstituted alkyl, substituted or unsubstituted heteroalkyl, L^{aa}-R^{6c}, C(O)R^{6c}, C(O)-L^{aa}-R^{6c}, NHC(O)R^{6c}
wherein
each R^{3″} is a member independently selected from H, substituted or unsubstituted alkyl and substituted or unsubstituted heteroalkyl;
L^{aa} is a member selected from a bond and a linker group; and
R^{6c} is a member selected from H, substituted or unsubstituted alkyl, substituted or unsubstituted heteroalkyl, substituted or unsubstituted aryl, substituted or unsubstituted heteroaryl, substituted or unsubstituted heterocycloalkyl, NR¹³R¹⁴ and a modifying group
wherein
R¹³ and R¹⁴ are members independently selected from H, substituted or unsubstituted alkyl and substituted or unsubstituted heteroalkyl.

16. The polypeptide conjugate of claim 15, wherein X* comprises a moiety according to Formula (III): 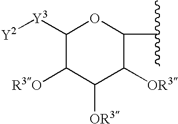
wherein Y³ is a member selected from CH and CH₂.

17. The polypeptide conjugate of claim 15, wherein X* comprises a moiety according to Formula (IV):

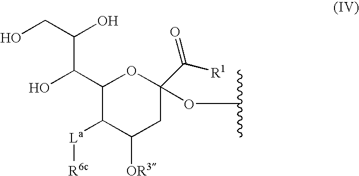

18. The polypeptide conjugate of claim 15, wherein at least one of R^{6b} and R^{6c} is a member selected from: 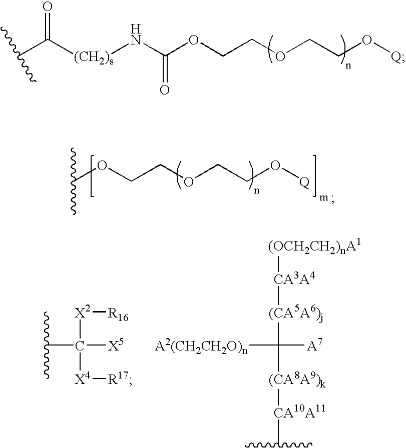
wherein
s, j and k are integers independently selected from 0 to 20;
each n is an integer independently selected from 0 to 2500;
m is an integer from 1-5;
Q is a member selected from H and C₁-C₆ alkyl;
R¹⁶ and R¹⁷ are independently selected polymeric moieties;
X² and X⁴ are independently selected linkage fragments joining polymeric moieties R¹⁶ and R¹⁷ to C; and
X⁵ is a non-reactive group;
A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NA¹²A¹³, —OA¹² and —SiA¹²A¹³
wherein
A¹² and A¹³ are members independently selected from substituted or unsubstituted alkyl, substituted or unsubstituted heteroalkyl, substituted or unsubstituted heterocycloalkyl, substituted or unsubstituted aryl, and substituted or unsubstituted heteroaryl.

19. A pharmaceutical composition comprising a polypeptide conjugate according to claim 1 and a pharmaceutically acceptable carrier, vehicle or diluent.

20. A method for making a polypeptide conjugate of claim 1, comprising the steps of:
(i) recombinantly producing said polypeptide, and
(ii) enzymatically glycosylating said polypeptide at said aromatic amino acid residue.


SEQUENCE LISTING (74 sequences)

SEQ ID NO: 1 - protein, 10 aa, Artificial Sequence
  glycosylation consensus sequence
WXXWXXWXXC

SEQ ID NO: 2 - protein, 4 aa, Artificial Sequence
  glycosylation consensus sequence
WSXW

SEQ ID NO: 3 - protein, 4 aa, Artificial Sequence
  glycosylation consensus sequence
WTXW

SEQ ID NO: 4 - protein, 4 aa, Artificial Sequence
  glycosylation consensus sequence
WAXW

SEQ ID NO: 5 - protein, 4 aa, Artificial Sequence
  glycosylation consensus sequence
WAQW

SEQ ID NO: 6 - protein, 5 aa, Artificial Sequence
  glycosylation consensus sequence
WSXWS

SEQ ID NO: 7 - protein, 5 aa, Artificial Sequence
  glycosylation consensus sequence
WSQWS

SEQ ID NO: 8 - protein, 4 aa, Artificial Sequence
  glycosylation consensus sequence
WSXC

SEQ ID NO: 9 - protein, 4 aa, Artificial Sequence
  glycosylation consensus sequence
WTXC

SEQ ID NO: 10 - protein, 7 aa, Artificial Sequence
  glycosylation consensus sequence
WSCWSSW

SEQ ID NO: 11 - protein, 7 aa, Artificial Sequence
  glycosylation consensus sequence
WGCWSSW

SEQ ID NO: 12 - protein, 12 aa, Artificial Sequence
  glycosylation consensus sequence
XWXXWXXWXXCX

SEQ ID NO: 13 - protein, 140 aa, Artificial Sequence
  human BMP-7
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 14 - protein, 140 aa, Artificial Sequence
  BMP-7 mutant
MWTQWKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 15 - protein, 140 aa, Artificial Sequence
  BMP-7 mutant
MSWTQWQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 16 - protein, 140 aa, Artificial Sequence
  BMP-7 mutant
MSTWTQWRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 17 - protein, 140 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRAWTQW

SEQ ID NO: 18 - protein, 144 aa, Artificial Sequence
  BMP-7 mutant
MWTQWSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 19 - protein, 143 aa, Artificial Sequence
  BMP-7 mutant
MWTQWTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 20 - protein, 142 aa, Artificial Sequence
  BMP-7 mutant
MWTQWGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 21 - protein, 142 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGWTQW

SEQ ID NO: 22 - protein, 143 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCWTQW

SEQ ID NO: 23 - protein, 144 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHWTQW

SEQ ID NO: 24 - protein, 142 aa, Artificial Sequence; identical to SEQ ID NO: 20
  BMP-7 mutant

SEQ ID NO: 25 - protein, 142 aa, Artificial Sequence
  BMP-7 mutant
MSWTQWSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 26 - protein, 142 aa, Artificial Sequence
  BMP-7 mutant
MSTWTQWKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 27 - protein, 142 aa, Artificial Sequence
  BMP-7 mutant
MSTGWTQWQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 28 - protein, 142 aa, Artificial Sequence
  BMP-7 mutant
MSTGSWTQWRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 29 - protein, 144 aa, Artificial Sequence
  BMP-7 mutant
MSWTQWTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 30 - protein, 144 aa, Artificial Sequence
  BMP-7 mutant
MSTWTQWGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 31 - protein, 144 aa, Artificial Sequence
  BMP-7 mutant
MSTGWTQWSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 32 - protein, 5 aa, Artificial Sequence
  oxygen glycosylation site sequence motif
WSQWS

SEQ ID NO: 33 - protein, 145 aa, Artificial Sequence
  BMP-7 mutant
MWSQWSSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 34 - protein, 144 aa, Artificial Sequence
  BMP-7 mutant
MWSQWSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 35 - protein, 143 aa, Artificial Sequence
  BMP-7 mutant
MWSQWSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 36 - protein, 142 aa, Artificial Sequence
  BMP-7 mutant
MWSQWSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 37 - protein, 141 aa, Artificial Sequence
  BMP-7 mutant
MWSQWSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 38 - protein, 145 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHWSQWS

SEQ ID NO: 39 - protein, 144 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCWSQWS

SEQ ID NO: 40 - protein, 143 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGWSQWS

SEQ ID NO: 41 - protein, 142 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACWSQWS

SEQ ID NO: 42 - protein, 141 aa, Artificial Sequence
  BMP-7 mutant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRAWSQWS

SEQ ID NO: 43 - protein, 4 aa, Artificial Sequence
  oxygen glycosylation site amino acid sequence motif
WTQW

SEQ ID NO: 44 - protein, 31 aa, Artificial Sequence
  partial sequence of BMP-7 substitution mutant
AFPLNSYMNATNHAIVQTLVHFINPETVPKP

SEQ ID NO: 45 - protein, 31 aa, Artificial Sequence
  partial sequence of BMP-7 substitution mutant
WTQWNSYMNATNHAIVQTLVHFINPETVPKP

SEQ ID NO: 46 - protein, 31 aa, Artificial Sequence
  partial sequence of BMP-7 substitution mutant
AWTQWSYMNATNHAIVQTLVHFINPETVPKP

SEQ ID NO: 47 - protein, 31 aa, Artificial Sequence
  partial sequence of BMP-7 substitution mutant
AFWTQWYMNATNHAIVQTLVHFINPETVPKP

SEQ ID NO: 48 - protein, 31 aa, Artificial Sequence
  partial sequence of BMP-7 substitution mutant
AFPWTQWMNATNHAIVQTLVHFINPETVPKP

SEQ ID NO: 49 - protein, 31 aa, Artificial Sequence
  partial sequence of BMP-7 substitution mutant
AFPLWTQWNATNHAIVQTLVHFINPETVPKP

SEQ ID NO: 50 - protein, 31 aa, Artificial Sequence
  partial sequence of BMP-7 substitution mutant
AFPLNWTQWATNHAIVQTLVHFINPETVPKP

SEQ ID NO: 51 - protein, 31 aa, Artificial Sequence
  partial sequence of BMP-7 substitution mutant
AFPLNSWTQWTNHAIVQTLVHFINPETVPKP

SEQ ID NO: 52 - protein, 31 aa, Artificial Sequence
  partial sequence of BMP-7 substitution mutant
AFPLNSYMNATNHAIVQTLVHFWTQWTVPKP

SEQ ID NO: 53 - protein, 31 aa, Artificial Sequence
  partial sequence of BMP-7 substitution mutant
AFPLNSYMNATNHAIVQTLVHFIWTQWVPKP

SEQ ID NO: 54 - protein, 31 aa, Artificial Sequence
  partial sequence of BMP-7 substitution mutant
AFPLNSYMNATNHAIVQTLVHFINWTQWPKP

SEQ ID NO: 55 - protein, 31 aa, Artificial Sequence
  partial sequence of BMP-7 substitution mutant
AFPLNSYMNATNHAIVQTLVHFINPWTQWKP

SEQ ID NO: 56 - protein, 31 aa, Artificial Sequence
  partial sequence of BMP-7 substitution mutant
AFPLNSYMNATNHAIVQTLVHFINPEWTQWP

SEQ ID NO: 57 - protein, 31 aa, Artificial Sequence
  partial sequence of BMP-7 substitution mutant
AFPLNSYMNATNHAIVQTLVHFINPETWTQW

SEQ ID NO: 58 - protein, 33 aa, Artificial Sequence
  partial sequence of BMP-7 insertion mutant
WTQWPLNSYMNATNHAIVQTLVHFINPETVPKP

SEQ ID NO: 59 - protein, 33 aa, Artificial Sequence
  partial sequence of BMP-7 insertion mutant
AWTQWLNSYMNATNHAIVQTLVHFINPETVPKP

SEQ ID NO: 60 - protein, 33 aa, Artificial Sequence
  partial sequence of BMP-7 insertion mutant
AFWTQWNSYMNATNHAIVQTLVHFINPETVPKP

SEQ ID NO: 61 - protein, 33 aa, Artificial Sequence
  partial sequence of BMP-7 insertion mutant
AFPWTQWSYMNATNHAIVQTLVHFINPETVPKP

SEQ ID NO: 62 - protein, 33 aa, Artificial Sequence
  partial sequence of BMP-7 insertion mutant
AFPLWTQWYMNATNHAIVQTLVHFINPETVPKP

SEQ ID NO: 63 - protein, 33 aa, Artificial Sequence
  partial sequence of BMP-7 insertion mutant
AFPLNWTQWMNATNHAIVQTLVHFINPETVPKP

SEQ ID NO: 64 - protein, 33 aa, Artificial Sequence
  partial sequence of BMP-7 insertion mutant
AFPLNSWTQWNATNHAIVQTLVHFINPETVPKP

SEQ ID NO: 65 - protein, 33 aa, Artificial Sequence
  partial sequence of BMP-7 insertion mutant
AFPLNSYWTQWATNHAIVQTLVHFINPETVPKP

SEQ ID NO: 66 - protein, 33 aa, Artificial Sequence
  partial sequence of BMP-7 insertion mutant
AFPLNSYMWTQWTNHAIVQTLVHFINPETVPKP

SEQ ID NO: 67 - protein, 33 aa, Artificial Sequence
  partial sequence of BMP-7 insertion mutant
AFPLNSYMNATNHAIVQTLVHFWTQWPETVPKP

SEQ ID NO: 68 - protein, 33 aa, Artificial Sequence
  partial sequence of BMP-7 insertion mutant
AFPLNSYMNATNHAIVQTLVHFIWTQWETVPKP

SEQ ID NO: 69 - protein, 33 aa, Artificial Sequence
  partial sequence of BMP-7 insertion mutant
AFPLNSYMNATNHAIVQTLVHFINWTQWTVPKP

SEQ ID NO: 70 - protein, 33 aa, Artificial Sequence
  partial sequence of BMP-7 insertion mutant
AFPLNSYMNATNHAIVQTLVHFINPWTQWVPKP

SEQ ID NO: 71 - protein, 33 aa, Artificial Sequence
  partial sequence of BMP-7 insertion mutant
AFPLNSYMNATNHAIVQTLVHFINPEWTQWPKP

SEQ ID NO: 72 - protein, 33 aa, Artificial Sequence
  partial sequence of BMP-7 insertion mutant
AFPLNSYMNATNHAIVQTLVHFINPETWTQWKP

SEQ ID NO: 73 - protein, 33 aa, Artificial Sequence
  partial sequence of BMP-7 insertion mutant
AFPLNSYMNATNHAIVQTLVHFINPETVWTQWP

SEQ ID NO: 74 - protein, 33 aa, Artificial Sequence
  partial sequence of BMP-7 insertion mutant
AFPLNSYMNATNHAIVQTLVHFINPETVPWTQW